(19) United States
(12) Patent Application Publication
(10) Pub. No.: US 2026/0027241 A1
(43) Pub. Date: Jan. 29, 2026

(54) Near-Infrared Dyes And Conjugates For Targeting Tumors

(71) Applicant: Lahjavida, Inc., Colorado Springs, CO (US)

(72) Inventors: Lyle D. Small, Colorado Springs, CO (US); Ruizheng Wang, Colorado Springs, CO (US)

(73) Assignee: Lahjavida, Inc., Colorado Springs, CO (US)

(21) Appl. No.: 19/343,829

(22) Filed: Sep. 29, 2025

Related U.S. Application Data
(63) Continuation-in-part of application No. 19/035,666, filed on Jan. 23, 2025.
Application No. 19/035,666 is a continuation-in-part of application No. 18/869,200.
(60) Provisional application No. 63/624,122, filed on Jan. 23, 2024.

(51) Int. Cl. A61K 51/04; A61K 47/54; A61P 35/00

(52) U.S. Cl. CPC A61K 51/0446; A61K 47/545; A61K 51/0497; A61P 35/00; A61K 2121/00

(57) ABSTRACT

A conjugate including a tumor-targeting NIR dye coupled to a therapeutic agent via a linker, whereby the dye functions to direct the conjugate and correspondingly, the therapeutic agent, to a target tumor cell population. Additionally, a method of killing tumor cells by directing a conjugate including a tumor-targeting NIR dye coupled to a therapeutic agent via a linker to a target tumor cell population.

### I. BACKGROUND

There remains a need for targeted cancer therapeutic agents with maximal efficacy in treating or killing tumor cells and minimal toxicity toward non-tumor or “normal” cells.

### II. SUMMARY OF THE INVENTION

Generally, the present invention details the use of tumor-targeting near-infrared (NIR) dyes in conjunction with a variety of therapeutic agents.

Following, a broad object of a particular embodiment of the invention can be to provide a conjugate including a tumor-targeting NIR dye coupled to a therapeutic agent via a linker, whereby the dye functions to direct the conjugate and correspondingly, the therapeutic agent, to a target tumor cell population.

Another broad object of a particular embodiment of the invention can be to provide a method of killing tumor cells by directing a conjugate including a tumor-targeting NIR dye coupled to a therapeutic agent via a linker to a target tumor cell population.

Naturally, further objects of the invention are disclosed throughout other areas of the specification, drawings, and claims.

### III. A BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows an illustrative example of a zwitterion which may be useful with the present invention when attached to a tumor-targeting NIR dye to form the inventive conjugate.

FIG. 1B shows an illustrative example of a zwitterion which may be useful with the present invention when attached to a tumor-targeting NIR dye to form the inventive conjugate.

FIG. 1C shows an illustrative example of a zwitterion which may be useful with the present invention when attached to a tumor-targeting NIR dye to form the inventive conjugate.

FIG. 1D shows an illustrative example of a zwitterion which may be useful with the present invention when attached to a tumor-targeting NIR dye to form the inventive conjugate.

FIG. 1E shows an illustrative example of a zwitterion which may be useful with the present invention when attached to a tumor-targeting NIR dye to form the inventive conjugate.

FIG. 1F shows an illustrative example of a zwitterion which may be useful with the present invention when attached to a tumor-targeting NIR dye to form the inventive conjugate.

FIG. 1G shows an illustrative example of a zwitterion which may be useful with the present invention when attached to a tumor-targeting NIR dye to form the inventive conjugate.

FIG. 1H shows an illustrative example of a zwitterion which may be useful with the present invention when attached to a tumor-targeting NIR dye to form the inventive conjugate.

FIG. 1I shows an illustrative example of a zwitterion which may be useful with the present invention when attached to a tumor-targeting NIR dye to form the inventive conjugate.

FIG. 1J shows an illustrative example of a zwitterion which may be useful with the present invention when attached to a tumor-targeting NIR dye to form the inventive conjugate.

FIG. 1K shows an illustrative example of a zwitterion which may be useful with the present invention when attached to a tumor-targeting NIR dye to form the inventive conjugate.

FIG. 2A shows an illustrative example of a chemotherapeutic agent having a linkable amine group which may be useful with the present invention when attached to a tumor-targeting NIR dye to form the inventive conjugate.

FIG. 2B shows an illustrative example of a chemotherapeutic agent having a linkable amine group which may be useful with the present invention when attached to a tumor-targeting NIR dye to form the inventive conjugate.

FIG. 2C shows an illustrative example of a chemotherapeutic agent having a linkable amine group which may be useful with the present invention when attached to a tumor-targeting NIR dye to form the inventive conjugate.

FIG. 2D shows an illustrative example of a chemotherapeutic agent having a linkable amine group which may be useful with the present invention when attached to a tumor-targeting NIR dye to form the inventive conjugate.

FIG. 2E shows an illustrative example of a chemotherapeutic agent having a linkable amine group which may be useful with the present invention when attached to a tumor-targeting NIR dye to form the inventive conjugate.

FIG. 2F shows an illustrative example of a chemotherapeutic agent having a linkable amine group which may be useful with the present invention when attached to a tumor-targeting NIR dye to form the inventive conjugate.

FIG. 2G shows an illustrative example of a chemotherapeutic agent having a linkable amine group which may be useful with the present invention when attached to a tumor-targeting NIR dye to form the inventive conjugate.

FIG. 2H shows an illustrative example of a chemotherapeutic agent having a linkable amine group which may be useful with the present invention when attached to a tumor-targeting NIR dye to form the inventive conjugate.

FIG. 2I shows an illustrative example of a chemotherapeutic agent having a linkable amine group which may be useful with the present invention when attached to a tumor-targeting NIR dye to form the inventive conjugate.

FIG. 2J shows an illustrative example of a chemotherapeutic agent having a linkable amine group which may be useful with the present invention when attached to a tumor-targeting NIR dye to form the inventive conjugate.

FIG. 3A shows an illustrative example of a chemotherapeutic agent having a linkable hydroxyl group which may be useful with the present invention when attached to a tumor-targeting NIR dye to form the inventive conjugate.

FIG. 3B shows an illustrative example of a chemotherapeutic agent having a linkable hydroxyl group which may be useful with the present invention when attached to a tumor-targeting NIR dye to form the inventive conjugate.

FIG. 3C shows an illustrative example of a chemotherapeutic agent having a linkable hydroxyl group which may be useful with the present invention when attached to a tumor-targeting NIR dye to form the inventive conjugate.

FIG. 4A shows an illustrative example of a chemotherapeutic agent having a linkable tertiary amine group which may be useful with the present invention when attached to a tumor-targeting NIR dye to form the inventive conjugate.

FIG. 4B shows an illustrative example of a chemotherapeutic agent having a linkable tertiary amine group which may be useful with the present invention when attached to a tumor-targeting NIR dye to form the inventive conjugate.

FIG. 4C shows an illustrative example of a chemotherapeutic agent having a linkable tertiary amine group which may be useful with the present invention when attached to a tumor-targeting NIR dye to form the inventive conjugate.

FIG. 4D shows an illustrative example of a chemotherapeutic agent having a linkable tertiary amine group which may be useful with the present invention when attached to a tumor-targeting NIR dye to form the inventive conjugate.

FIG. 4E shows an illustrative example of a chemotherapeutic agent having a linkable tertiary amine group which may be useful with the present invention when attached to a tumor-targeting NIR dye to form the inventive conjugate.

FIG. 4F shows an illustrative example of a chemotherapeutic agent having a linkable tertiary amine group which may be useful with the present invention when attached to a tumor-targeting NIR dye to form the inventive conjugate.

FIG. 4G shows an illustrative example of a chemotherapeutic agent having a linkable tertiary amine group which may be useful with the present invention when attached to a tumor-targeting NIR dye to form the inventive conjugate.

FIG. 4H shows an illustrative example of a chemotherapeutic agent having a linkable tertiary amine group which may be useful with the present invention when attached to a tumor-targeting NIR dye to form the inventive conjugate.

FIG. 4I shows an illustrative example of a chemotherapeutic agent having a linkable tertiary amine group which may be useful with the present invention when attached to a tumor-targeting NIR dye to form the inventive conjugate.

FIG. 4J shows an illustrative example of a chemotherapeutic agent having a linkable tertiary amine group which may be useful with the present invention when attached to a tumor-targeting NIR dye to form the inventive conjugate.

FIG. 4K shows an illustrative example of a chemotherapeutic agent having a linkable tertiary amine group which may be useful with the present invention when attached to a tumor-targeting NIR dye to form the inventive conjugate.

FIG. 4L shows an illustrative example of a chemotherapeutic agent having a linkable secondary amine group which may be useful with the present invention when attached to a tumor-targeting NIR dye to form the inventive conjugate.

FIG. 4M shows an illustrative example of a chemotherapeutic agent having a linkable secondary amine group which may be useful with the present invention when attached to a tumor-targeting NIR dye to form the inventive conjugate.

FIG. 5 shows an embodiment of the inventive conjugate.

FIG. 6A shows a tumor-targeting NIR dye coupled to a chemotherapeutic agent via a linker including a cleavable portion with acid sensitivity.

FIG. 6B shows a tumor-targeting NIR dye coupled to a chemotherapeutic agent via a linker including a cleavable portion with acid sensitivity.

FIG. 7A shows an embodiment of the inventive conjugate.

FIG. 7B shows an embodiment of the inventive conjugate.

FIG. 7C shows an embodiment of the inventive conjugate.

FIG. 7D shows an embodiment of the inventive conjugate.

FIG. 7E shows an embodiment of the inventive conjugate.

FIG. 7F shows an embodiment of the inventive conjugate.

FIG. 8A shows an illustrative example of a protease-sensitive cleavable peptide portion which may be useful with the present invention to link a tumor-targeting NIR dye and a chemotherapeutic agent to form the inventive conjugate.

FIG. 8B shows an illustrative example of a protease-sensitive cleavable peptide portion which may be useful with the present invention to link a tumor-targeting NIR dye and a chemotherapeutic agent to form the inventive conjugate.

FIG. 8C shows an illustrative example of a protease-sensitive cleavable peptide portion which may be useful with the present invention to link a tumor-targeting NIR dye and a chemotherapeutic agent to form the inventive conjugate.

FIG. 8D shows an illustrative example of a protease-sensitive cleavable peptide portion which may be useful with the present invention to link a tumor-targeting NIR dye and a chemotherapeutic agent to form the inventive conjugate.

FIG. 8E shows an illustrative example of a protease-sensitive cleavable peptide portion which may be useful with the present invention to link a tumor-targeting NIR dye and a chemotherapeutic agent to form the inventive conjugate.

FIG. 8F shows an illustrative example of a protease-sensitive cleavable peptide portion which may be useful with the present invention to link a tumor-targeting NIR dye and a chemotherapeutic agent to form the inventive conjugate.

FIG. 8G shows an illustrative example of a protease-sensitive cleavable peptide portion which may be useful with the present invention to link a tumor-targeting NIR dye and a chemotherapeutic agent to form the inventive conjugate.

FIG. 8H shows an illustrative example of a protease-sensitive cleavable peptide portion which may be useful with the present invention to link a tumor-targeting NIR dye and a chemotherapeutic agent to form the inventive conjugate.

FIG. 8I shows an illustrative example of a protease-sensitive cleavable peptide portion which may be useful with the present invention to link a tumor-targeting NIR dye and a chemotherapeutic agent to form the inventive conjugate.

FIG. 8J shows an illustrative example of a protease-sensitive cleavable portion which may be useful with the present invention to link a tumor-targeting NIR dye and a chemotherapeutic agent to form the inventive conjugate.

FIG. 8K shows an illustrative example of a protease-sensitive cleavable portion which may be useful with the present invention to link a tumor-targeting NIR dye and a chemotherapeutic agent to form the inventive conjugate.

FIG. 8L shows an illustrative example of a protease-sensitive cleavable portion which may be useful with the present invention to link a tumor-targeting NIR dye and a chemotherapeutic agent to form the inventive conjugate.

FIG. 8M shows an illustrative example of a protease-sensitive cleavable portion which may be useful with the present invention to link a tumor-targeting NIR dye and a chemotherapeutic agent to form the inventive conjugate.

FIG. 8N shows an illustrative example of a protease-sensitive cleavable peptide portion which may be useful with the present invention to link a tumor-targeting NIR dye and a chemotherapeutic agent to form the inventive conjugate.

FIG. 9A shows a tumor-targeting NIR dye coupled to a chemotherapeutic agent via a linker including a cleavable portion with enzyme sensitivity.

FIG. 9B shows a tumor-targeting NIR dye coupled to a chemotherapeutic agent via a linker including a cleavable portion with enzyme sensitivity.

FIG. 9C shows a tumor-targeting NIR dye coupled to a chemotherapeutic agent via a linker including a cleavable portion with enzyme sensitivity.

FIG. 9D shows a tumor-targeting NIR dye coupled to a chemotherapeutic agent via a linker including a cleavable portion with enzyme sensitivity.

FIG. 9E shows a tumor-targeting NIR dye coupled to a chemotherapeutic agent via a linker including a cleavable portion with enzyme sensitivity.

FIG. 9F shows a tumor-targeting NIR dye coupled to a chemotherapeutic agent via a linker including a cleavable portion with enzyme sensitivity.

FIG. 10A shows a tumor-targeting NIR dye coupled to a chemotherapeutic agent via a linker including a cleavable portion with enzyme sensitivity.

FIG. 10B shows a tumor-targeting NIR dye coupled to a chemotherapeutic agent via a linker including a cleavable portion with enzyme sensitivity.

FIG. 11 shows a tumor-targeting NIR dye coupled to a chemotherapeutic agent via a linker including a cleavable portion with enzyme sensitivity.

FIG. 12A shows an illustrative example of a first self-immolative portion of a linker which may be useful with the present invention.

FIG. 12B shows an illustrative example of a first self-immolative portion of a linker which may be useful with the present invention.

FIG. 13 shows an illustrative example of a second self-immolative portion of a linker which may be useful with the present invention.

FIG. 14 shows an illustrative example of a hydrophilic portion of a linker which may be useful with the present invention.

FIG. 15 shows an illustrative example of a click chemistry reagent which may be useful with the present invention.

FIG. 16 shows a compound which can function as a base which may accept a proton (H⁺).

FIG. 17A shows an illustrative example of a basic functional group which may be useful with the present invention.

FIG. 17B shows an illustrative example of a basic functional group which may be useful with the present invention.

FIG. 17C shows an illustrative example of a basic functional group which may be useful with the present invention.

FIG. 17D shows an illustrative example of a basic functional group which may be useful with the present invention.

FIG. 17E shows an illustrative example of a basic functional group which may be useful with the present invention.

FIG. 17F shows an illustrative example of a basic functional group which may be useful with the present invention.

FIG. 18A shows an illustrative example of a mitochondria-targeting functional group which may be useful with the present invention.

FIG. 18B shows an illustrative example of a mitochondria-targeting functional group which may be useful with the present invention.

FIG. 18C shows an illustrative example of a mitochondria-targeting functional group which may be useful with the present invention.

FIG. 18D shows an illustrative example of a mitochondria-targeting functional group which may be useful with the present invention.

FIG. 19A shows an illustrative example of a functional group which can facilitate noncovalent interactions between a tumor-targeting NIR dye and albumin and thus may be useful with the present invention.

FIG. 19B shows an illustrative example of a functional group which can facilitate noncovalent interactions between a tumor-targeting NIR dye and albumin and thus may be useful with the present invention.

FIG. 20 shows an embodiment of the inventive conjugate.

FIG. 21A shows an illustrative example of a chelator which may be useful with the present invention.

FIG. 21B shows an illustrative example of a chelator which may be useful with the present invention.

FIG. 21C shows an illustrative example of a chelator which may be useful with the present invention.

FIG. 21D shows an illustrative example of a chelator which may be useful with the present invention.

FIG. 22 shows an embodiment of the inventive conjugate.

FIG. 23A shows an illustrative example of a molecular glue which may be useful with the present invention.

FIG. 23B shows an illustrative example of a molecular glue which may be useful with the present invention.

FIG. 23C shows an illustrative example of a molecular glue which may be useful with the present invention.

FIG. 24 shows conjugation of a tumor-targeting NIR dye to DBCO.

FIG. 25 shows conjugation of a tumor-targeting NIR dye to PEG.

FIG. 26 shows an exemplary synthesis of the conjugate shown in Formula XXII.

FIG. 27 shows an exemplary synthesis of the conjugate shown in Formula XXXIV.

FIG. 28 shows an exemplary synthesis of the conjugate shown in Formula LXXII.

FIG. 29A shows NIR imaging of a mouse bearing a SW620 tumor on its right flank at 24 hours post dose of the conjugate shown in Formula XXII.

FIG. 29B shows NIR imaging of the mouse shown in FIG. 29A 4 days post conjugate dose.

FIG. 29C shows NIR imaging of the mouse shown in FIG. 29A 9 days post conjugate dose.

FIG. 30A shows NIR imaging of a mouse bearing a SW620 tumor on its right flank at 24 hours post dose of the conjugate shown in Formula XLVI.

FIG. 30B shows NIR imaging of the mouse shown in FIG. 30A 4 days post conjugate dose.

FIG. 30C shows NIR imaging of the mouse shown in FIG. 30A 9 days post conjugate dose.

FIG. 31 shows the efficacy of (i) doxorubicin HCl and (ii) the conjugate shown in Formula XXII in mice bearing A549 tumors on their right flanks.

FIG. 32A shows the toxicity of doxorubicin in the mice of FIG. 31.

FIG. 32B shows the toxicity of the conjugate shown in Formula XXII in the mice of FIG. 31.

### IV. DETAILED DESCRIPTION OF THE PREFERRED EMBODIMENTS

The present invention provides an effective and versatile delivery method which combines tumor-targeting NIR dyes with various cancer therapeutic agents to create conjugates for (i) targeting tumor cells and/or (ii) killing tumor cells and/or (iii) imaging tumor cells and/or (iv) theranostics, whereby the dye functions to direct the conjugate and correspondingly, the therapeutic agent, to a target tumor cell population; this can be in contrast to the non-specific delivery of conventional anticancer agents which may cause significant adverse side effects. In addition to targeting tumor cells, the present dye can absorb NIR light and emit in the same region with appreciable brightness for corresponding detection and imaging/visualization of tumor location; thus, the dye can function as an optical reporter which may enable real-time monitoring of the delivery and release of the therapeutic agent. Such a property can be extremely desirable considering the noninvasive nature of NIR light and its tissue penetration, which may be orders of magnitude greater than that for ultraviolet or visible light.

As to particular embodiments, the dye can be a cyanine dye, whereby chemically, cyanine dyes include two nitrogen atoms joined by a polymethine chain.

As to particular embodiments, the dye can be a heptamethine cyanine dye.

As to particular embodiments, the dye can have Formula I as follows:

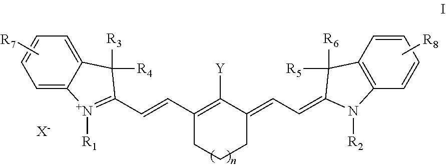

wherein:
R₁ to R₈ can each be independently an unsubstituted or substituted C1 to C10 alkyl group, an unsubstituted or substituted aryl group, an unsubstituted or substituted C1 to C10 alkoxyl group, an alkylsulfonate, an alkyl carboxylic acid group, or an alkylamino group;
X can be Br⁻, Cl⁻, I⁻, perchlorate (ClO₄⁻), tosylate (OTs), or absent if another covalently linked anion moiety is present in the dye molecule;
Y can be Cl, or a substituted C, O, S, or N group; and
n can be 0 (such as for a five-membered ring) or 1 (such as for a six-membered ring).

Exemplary dyes which may be useful with the present invention are shown in Formulas II through V.

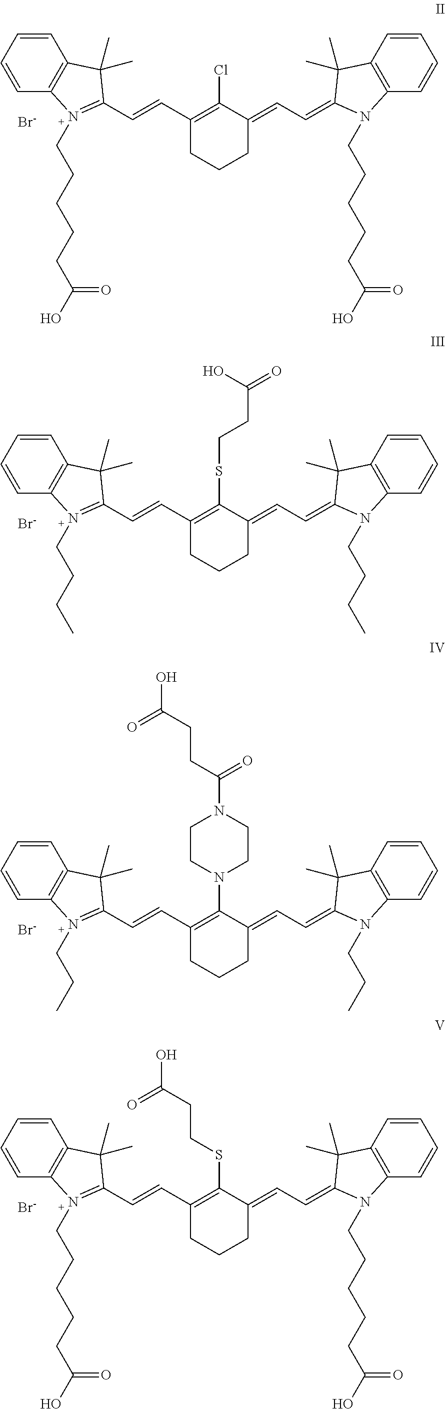

As to particular embodiments, the dye can be modified to have zwitterionic functionality, whereby such a modification may include the attachment of one or more zwitterions to the dye or the incorporation of one or more zwitterions into the dye, each said zwitterion functioning as a polyelectrolyte or dipolar ion which has both positively and negatively charged groups, yet can be overall neutral in charge. Additionally, zwitterions can have a high hydration capacity and correspondingly may be highly hydrated via electrostatic interactions of their positively and negatively charged groups with polar water molecules, whereby the resultant tightly bound water layer forms a dense and stable hydration shell or solvation shell which can provide a physical and energetic barrier that may prevent undesired adsorption, such as but not limited to protein adsorption; thus, zwitterions can have effective antifouling properties.

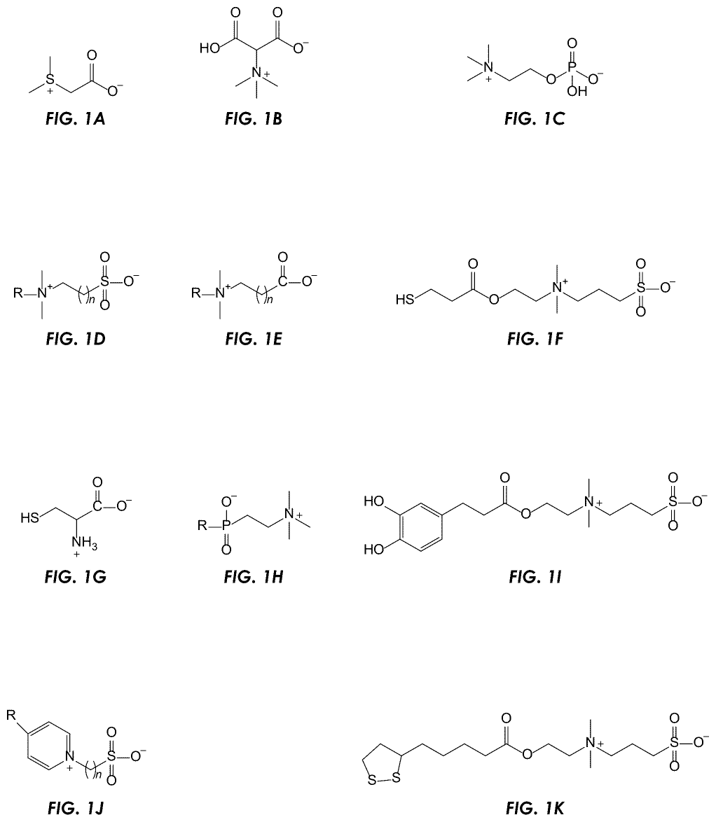

As to particular embodiments, illustrative examples of zwitterions which may be useful with the present invention when attached to or incorporated into the present dye include those shown in FIGS. 1A through 1K. In line with the above, the zwitterions shown in these figures can be overall neutrally charged and have remarkable hydration capabilities via strong ion-dipole interactions of their positively and negatively charged groups with polar water molecules.

To impart zwitterionic functionality to the dye, a zwitterion can be covalently or electrostatically attached or bound to the dye via the central ring (such as the central cyclopentyl or cyclohexyl ring), via the nitrogen of an indole moiety, via a geminal position, or via an aromatic ring. As to particular embodiments, one or more of R₁ to R₈ or Y as shown in Formula I can be functionalized with a zwitterion and/or used as a linking site to attach or bind a zwitterion to the dye.

Exemplary dyes having zwitterionic functionality which may be useful with the present invention are shown in Formulas VI through XV.

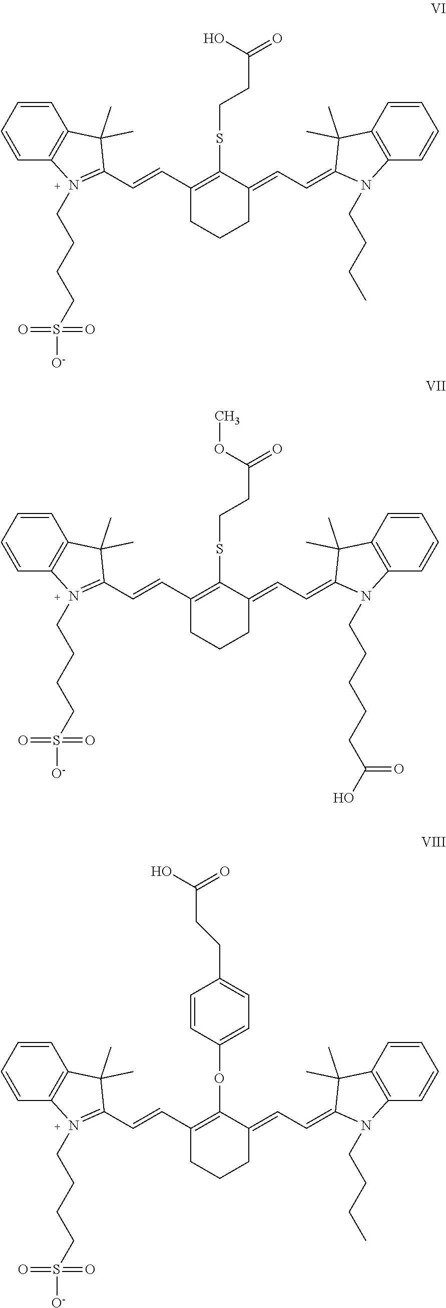

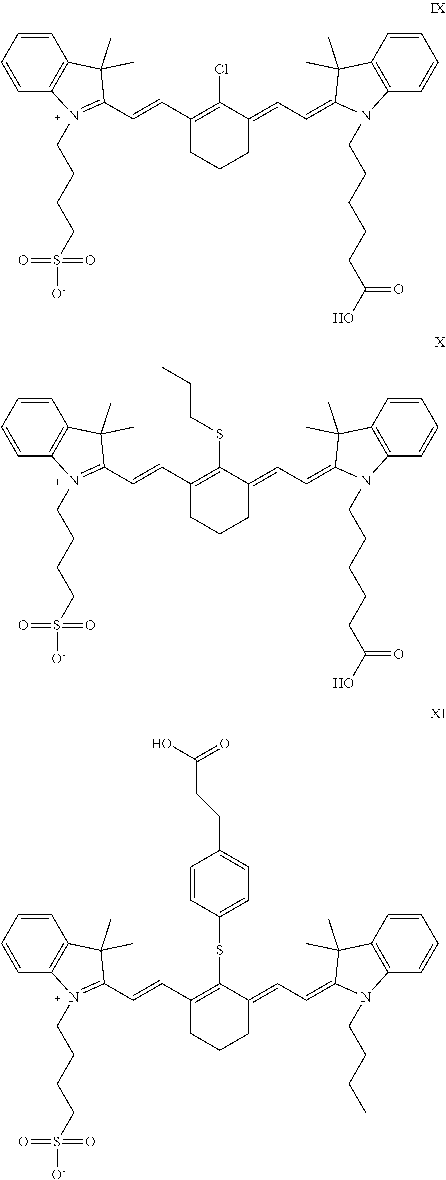

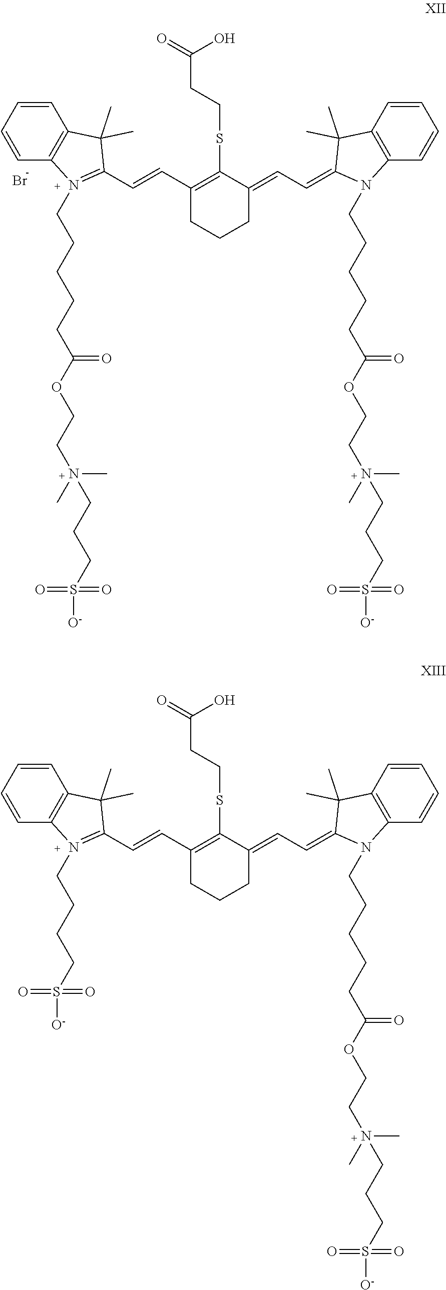

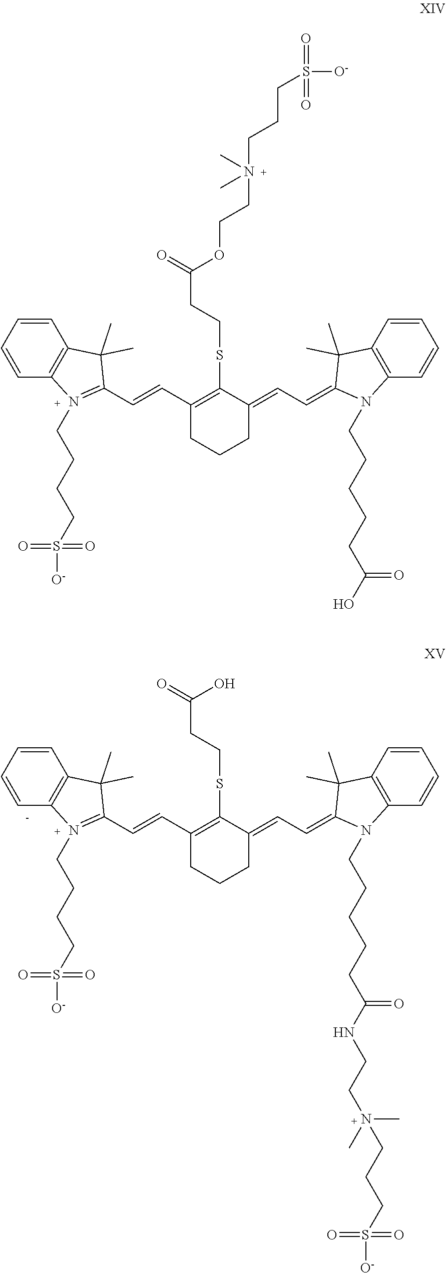

As stated above, the present invention can combine a tumor-targeting NIR dye with various therapeutic agents to create conjugates for the treatment of different cancers, whereby these conjugates may be delivered directly to a target tumor cell population. To form such conjugates, a payload, such as a therapeutic agent, can be covalently or electrostatically attached or bound to the dye, for example via the central ring (such as the central cyclopentyl or cyclohexyl ring), via the nitrogen of an indole moiety, via a geminal position, or via an aromatic ring. As to particular embodiments, one or more of R₁ to R₈ or Y as shown in Formula I can be used as a linking site to attach or bind the payload to the dye via various linkers to form the present conjugate.

As to particular embodiments, a dye can be coupled to (for example via conjugation) a payload comprising a chemotherapeutic agent via various linkers for use in chemotherapy, as per the present invention. As used herein, the term “chemotherapeutic agent” can mean a chemical agent, and the term “chemotherapy” can mean the therapeutic use of such a chemical agent.

As a first example, chemotherapeutic agents which may be conjugated to a tumor-targeting NIR dye as per the present invention can include, but are not limited to, DNA damaging agents, tubulin inhibitors, and growth factor inhibitors (such as inhibitors of epidermal growth factor (EGF), vascular endothelial growth factor (VEGF), platelet-derived endothelial growth factor (PDGF), fibroblast growth factor (FGF), human epidermal growth factor receptor 2 (HER2), or the like).

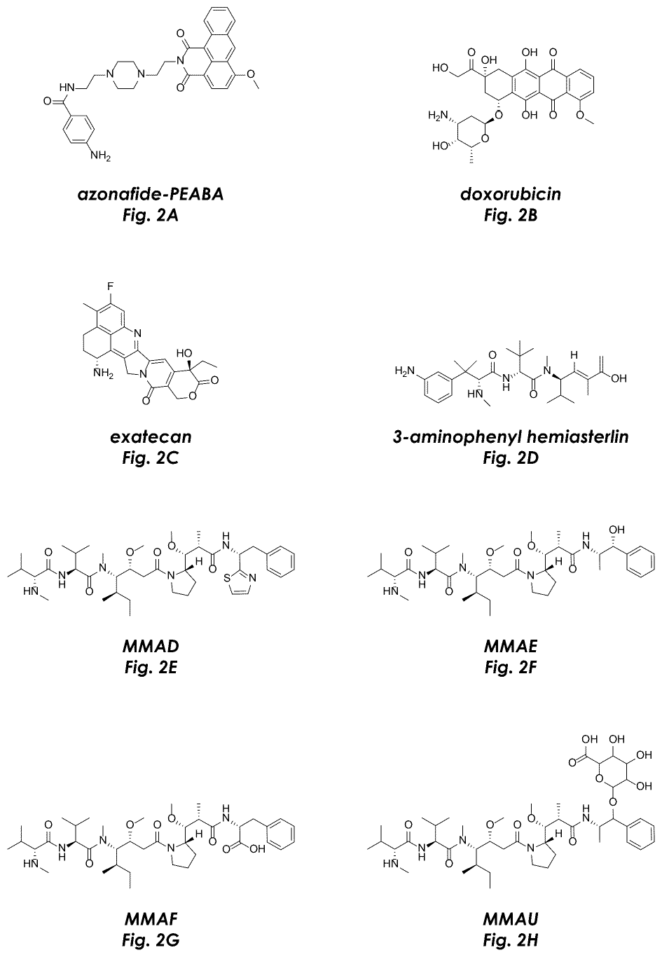

As a second example, chemotherapeutic agents including a linkable amine group which may be conjugated to a tumor-targeting NIR dye as per the present invention can include those shown in FIGS. 2A through 2J.

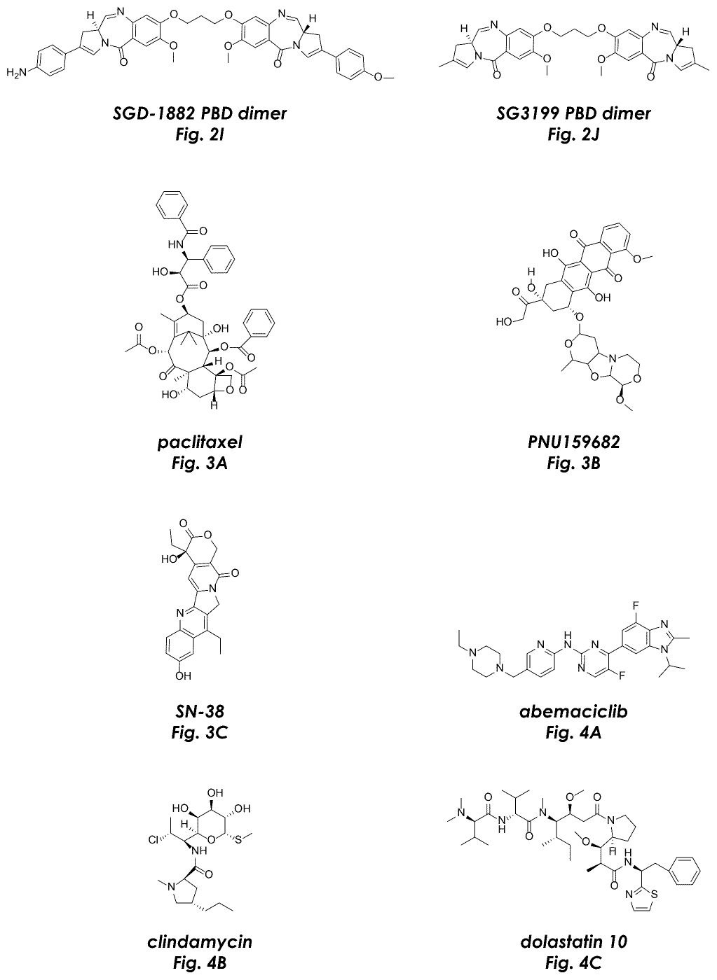

As a third example, chemotherapeutic agents including a linkable hydroxyl group which may be conjugated to a tumor-targeting NIR dye as per the present invention can include those shown in FIGS. 3A through 3C.

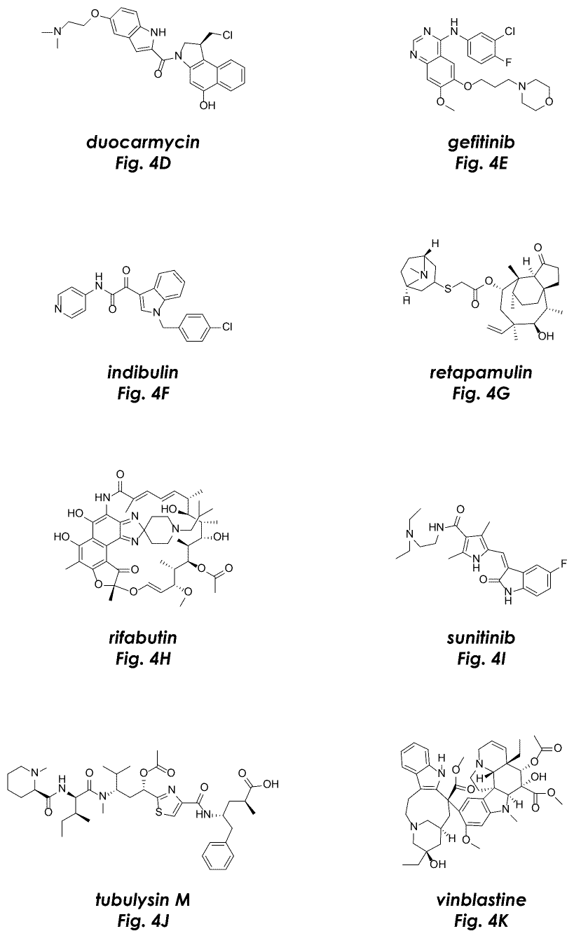

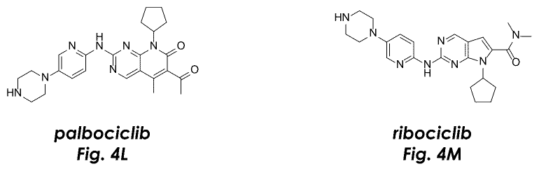

As a fourth example, chemotherapeutic agents including a linkable tertiary amine group which may be conjugated to a tumor-targeting NIR dye as per the present invention can include those shown in FIGS. 4A through 4K.

As a fifth example, chemotherapeutic agents including a linkable secondary amine group, such as in a piperazine ring, which may be conjugated to a tumor-targeting NIR dye as per the present invention can include those shown in FIGS. 4L and 4M.

As a sixth example, chemotherapeutic agents which may be conjugated to a tumor-targeting NIR dye as per the present invention can include abemaciclib, actinomycin, all-trans retinoic acid, azacitidine, azathioprine, azonafide, azonafide-PEABA, bleomycin, bortezomib, carboplatin, capecitabine, cisplatin, chlorambucil, clindamycin, cyclophosphamide, cytarabine, daunorubicin, dolastatin 10, doxifluridine, doxorubicin, duocarmycin, epirubicin, epothilone, etoposide, exatecan, fluorouracil, gefitinib, gemcitabine, hemiasterlin, 3-aminophenyl hemiasterlin, hydroxyurea, idarubicin, imatinib, indibulin, irinotecan, matrine, monomethyl auristatin E (MMAE), monomethyl auristatin F (MMAF), monomethyl auristatin U (MMAU), mechlorethamine, methotrexate, mitoxantrone, monomethyl dolastatin 10 (MMAD), nitrogen mustard, oxaliplatin, paclitaxel, palbociclib, pemetrexed, PNU159682, pyrrolobenzodiazepines (PBD), PBD dimers, retapamulin, ribociclib, rifabutin, SG3199 PBD dimer, SN-38, sunitinib, teniposide, tioguanine, topotecan, tubulysin A, tubulysin B, tubulysin D, tubulysin M, tubulysin U, tubulysin V, valrubicin, vemurafenib, vinblastine, vincristine, vindesine, or the like.

For use with the present invention, the chemotherapeutic agent can be fully pharmacologically active following conjugation or alternatively, the chemotherapeutic agent may be a prodrug, meaning an inactive or less active derivative of the chemotherapeutic agent which can be transformed into its pharmacologically active form via metabolism, such as by an enzymatic and/or chemical mechanism, whereby the pharmacologically active form may be effective to kill tumor cells.

In contrast to administration in its pharmacologically active form, administering a chemotherapeutic agent in its prodrug form can improve the absorption, distribution, metabolism, and/or excretion (ADME) of said chemotherapeutic agent. For example, a prodrug form can have improved bioavailability relative to its pharmacologically active form, which may be beneficial for chemotherapeutic agents that are poorly absorbed via the gastrointestinal tract. Additionally, the prodrug form can facilitate how selectively the chemotherapeutic agent interacts with cells or processes that are not its intended target, which may reduce unintended, undesirable, and/or adverse side effects of the chemotherapeutic agent.

As stated above, the present dye can be coupled to (for example via conjugation) a chemotherapeutic agent via various linkers for use in chemotherapy, whereby the specific linker which conjugates the two compounds may contribute to a decrease in the adverse side effects of the chemotherapeutic agent, as the linker can affect the pharmacokinetics, pharmacodynamics, stability, toxicity, etc. of the chemotherapeutic agent incorporated into the conjugate. A preferred linker can ensure adequate stability of the chemotherapeutic agent in the circulation, effectively prevent premature release of the chemotherapeutic agent proximate an unintended target, and use the inherent properties of tumor cells and/or the tumor microenvironment to facilitate the release and/or pharmacological activation of the chemotherapeutic agent only proximate a target tumor cell population.

As to particular embodiments, a linker comprising a cleavable portion can be useful to conjugate a tumor-targeting NIR dye and a chemotherapeutic agent as per the present invention.

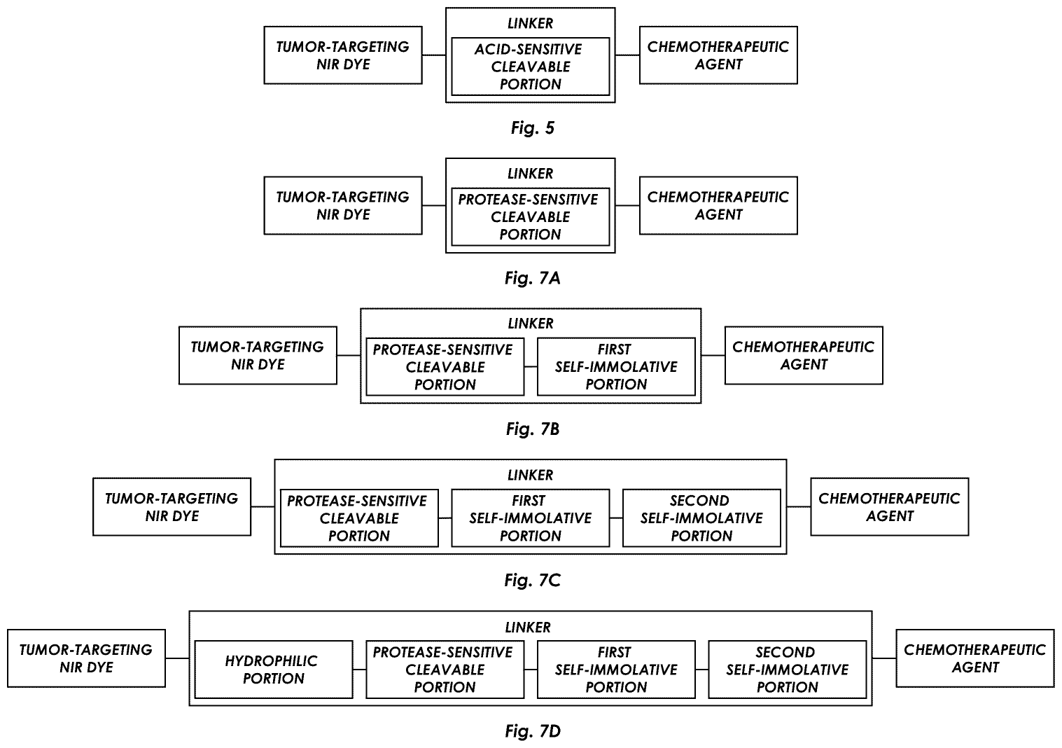

Now referring primarily to FIG. 5, as to particular embodiments, the linker can comprise a cleavable portion with pH sensitivity, whereby acid-sensitive cleavable portions may exploit the acidic microenvironment of tumors to trigger the hydrolysis of an acid-labile group(s) within the acid-sensitive cleavable portion to facilitate the release of the chemotherapeutic agent only proximate a target tumor cell population.

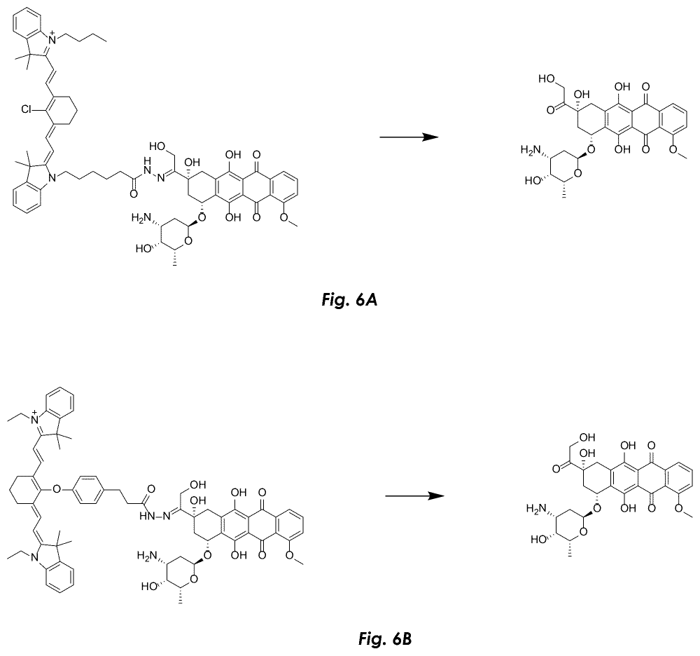

As to particular embodiments, a cleavable portion with acid sensitivity can comprise an acid-labile hydrazone bond (as shown in FIGS. 6A and 6B).

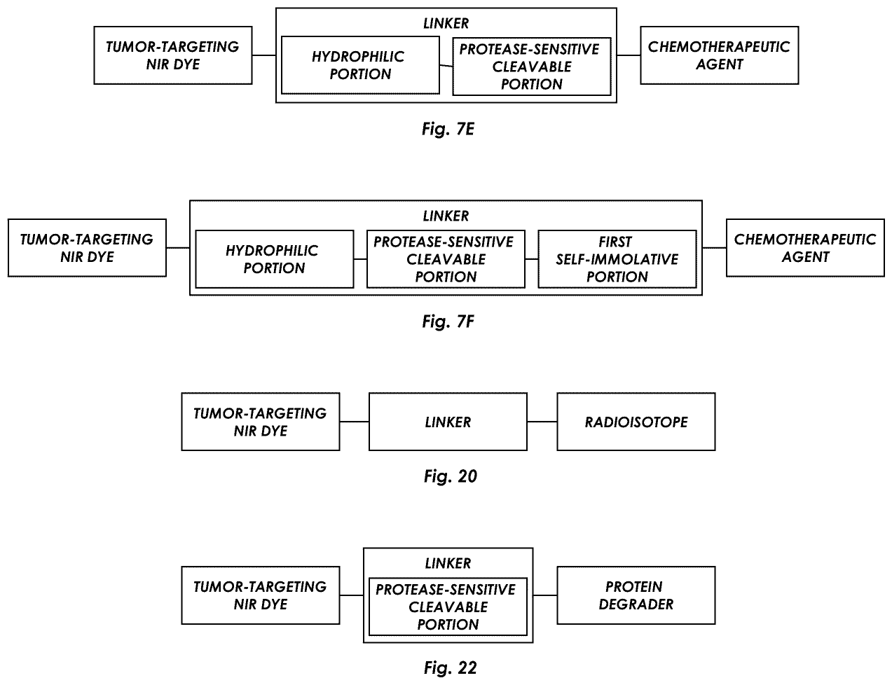

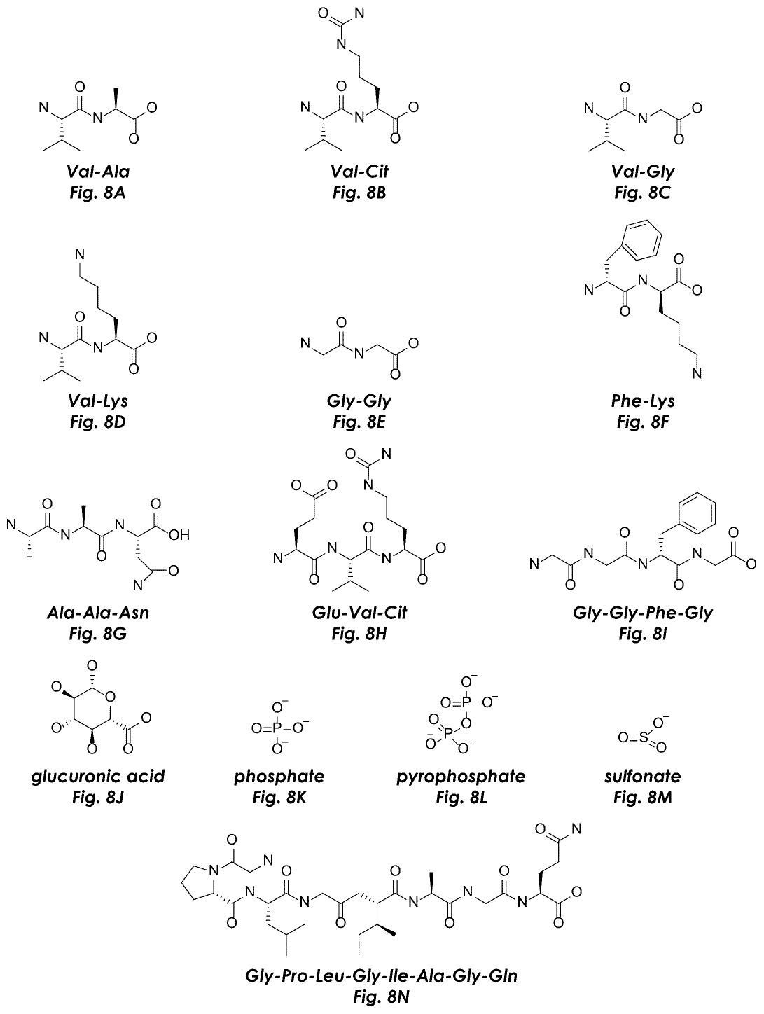

Now referring primarily to FIGS. 7A through 7F, as to particular embodiments, the linker can comprise a cleavable portion with enzyme sensitivity (i.e., an enzymatically-cleavable portion), such as a cleavable portion with protease sensitivity. In use, a protease found in tumor cells and/or in the tumor microenvironment can recognize and cleave a specific substrate to advantageously release a linked payload only proximate a target tumor cell population. Further, protease-sensitive cleavable portions can be stable in the systemic circulation due to the presence of protease inhibitors in the blood. As to particular embodiments, illustrative examples of protease-sensitive cleavable portions which may be useful with the present invention include those shown in FIGS. 8A through 8N.

As to particular embodiments, the linker can comprise a cleavable peptide portion with protease sensitivity. In use, a protease found in tumor cells and/or in the tumor microenvironment can recognize and cleave a specific peptide sequence in a cleavable peptide portion to advantageously release a linked payload only proximate a target tumor cell population.

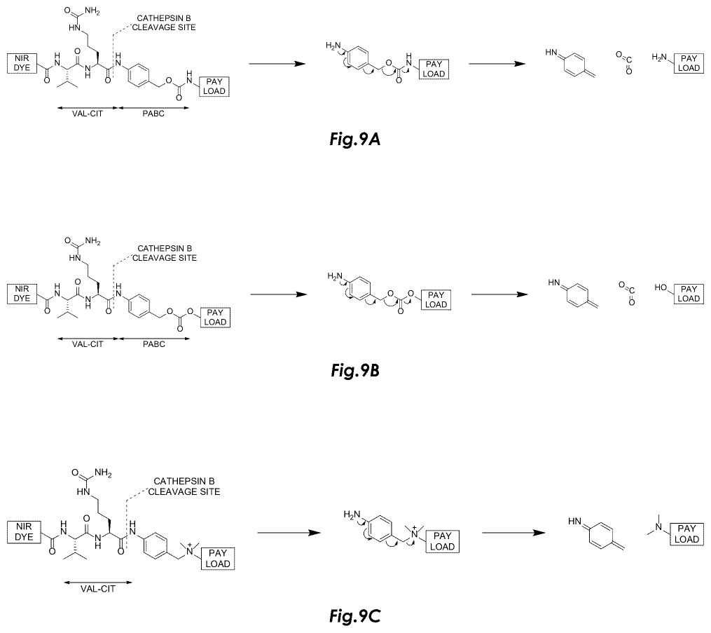

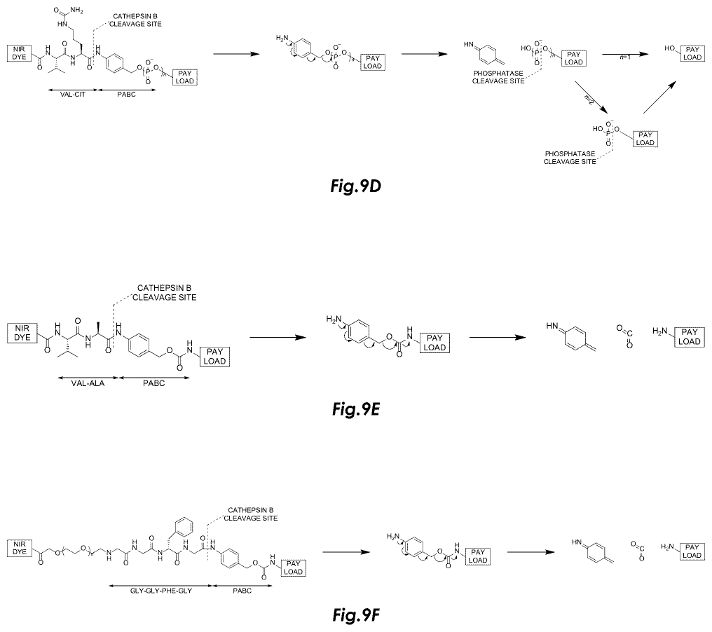

As to particular embodiments, a cleavable peptide portion with protease sensitivity can comprise a peptide sequence which may be cleaved by cathepsin B, a lysosomal protease which may be overexpressed in and around tumor cells. As illustrative examples, cathepsin B-sensitive peptide sequences can include a valine-citrulline (VC) dipeptide (as shown in FIGS. 8B and 9A through 9D), a valine-alanine (VA) dipeptide (as shown in FIGS. 8A and 9E), a phenylalanine-lysine (FK) dipeptide (as shown in FIG. 8F), and a glycine-glycine-phenylalanine-glycine (GGFG) tetrapeptide (as shown in FIGS. 81 and 9F).

As to particular embodiments, a cleavable peptide portion with protease sensitivity can comprise a peptide sequence which may be cleaved by matrix metalloproteinase 2 (MMP-2), an enzyme which breaks down components of the extracellular matrix and may be overexpressed in and around tumor cells. As an illustrative example, an MMP-2-sensitive peptide sequence can include the synthetic octapeptide glycine-proline-leucine-glycine-isoleucine-alanine-glycine-glutamine (GPLGIAGQ) (as shown in FIG. 8N).

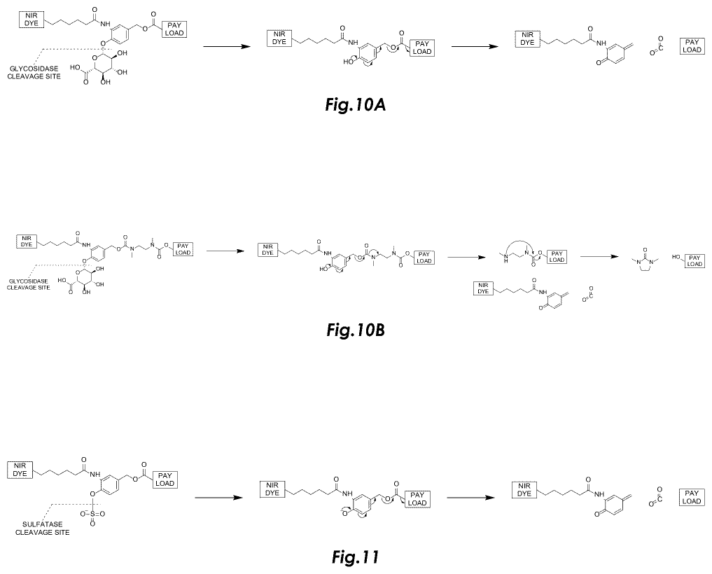

As to particular embodiments, a cleavable portion with protease sensitivity can comprise glucuronic acid (as shown in FIGS. 8N, 10A, and 10B) which may be cleaved by β-glucuronidase, a glycosidase which breaks down complex carbohydrates and may be overexpressed in and around tumor cells. Of note, glucuronic acid-containing cleavable portions can have relatively high water solubility.

As to particular embodiments, a cleavable portion with protease sensitivity can comprise a phosphatase-sensitive phosphate (as shown in FIGS. 8K and 9D) or a pyrophosphatase-sensitive pyrophosphate (as shown in FIG. 8L), whereby phosphatase and pyrophosphatase catalyze the hydrolysis of phosphate and pyrophosphate, respectively. Of note, phosphate-containing cleavable portions and pyrophosphate-containing cleavable portions can have relatively high water solubility.

As to particular embodiments, a cleavable portion with protease sensitivity can comprise a sulphatase-sensitive sulphonate (as shown in FIGS. 8M and 11), whereby a sulphatase catalyzes the hydrolysis of a sulfate ester. Of note, sulphonate-containing cleavable portions can have relatively high water solubility.

Exemplary conjugates comprising a tumor-targeting NIR dye and specifically, the dye shown in Formula VI, coupled to a chemotherapeutic agent via a linker including a cleavable peptide portion which may be useful with the present invention are shown in Formulas XVI through LIII.

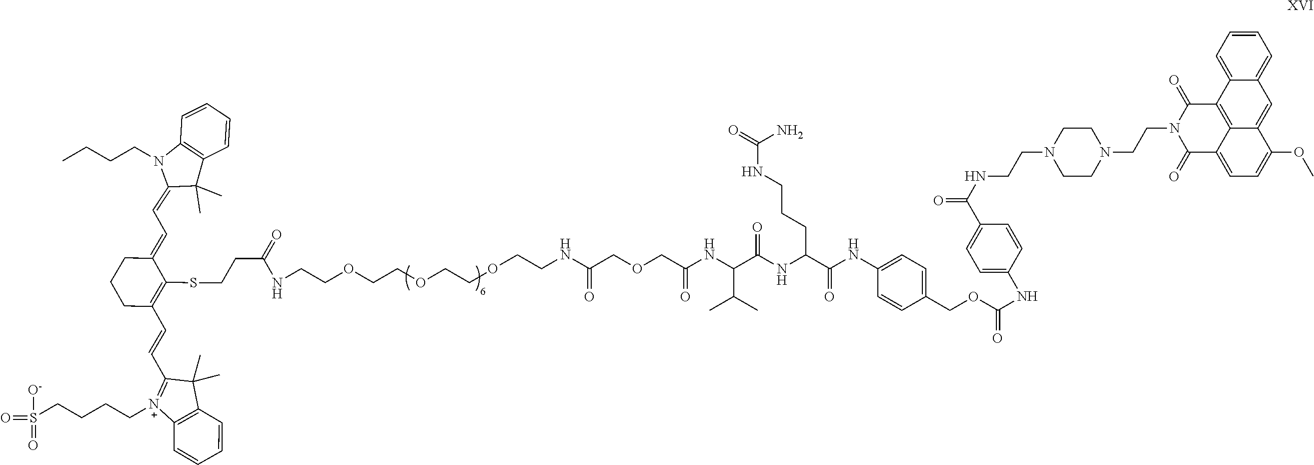

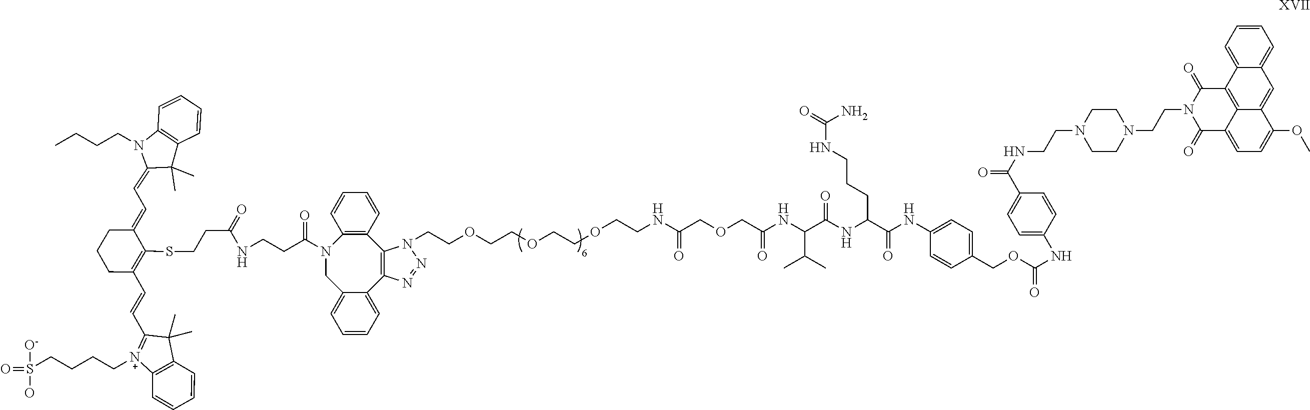

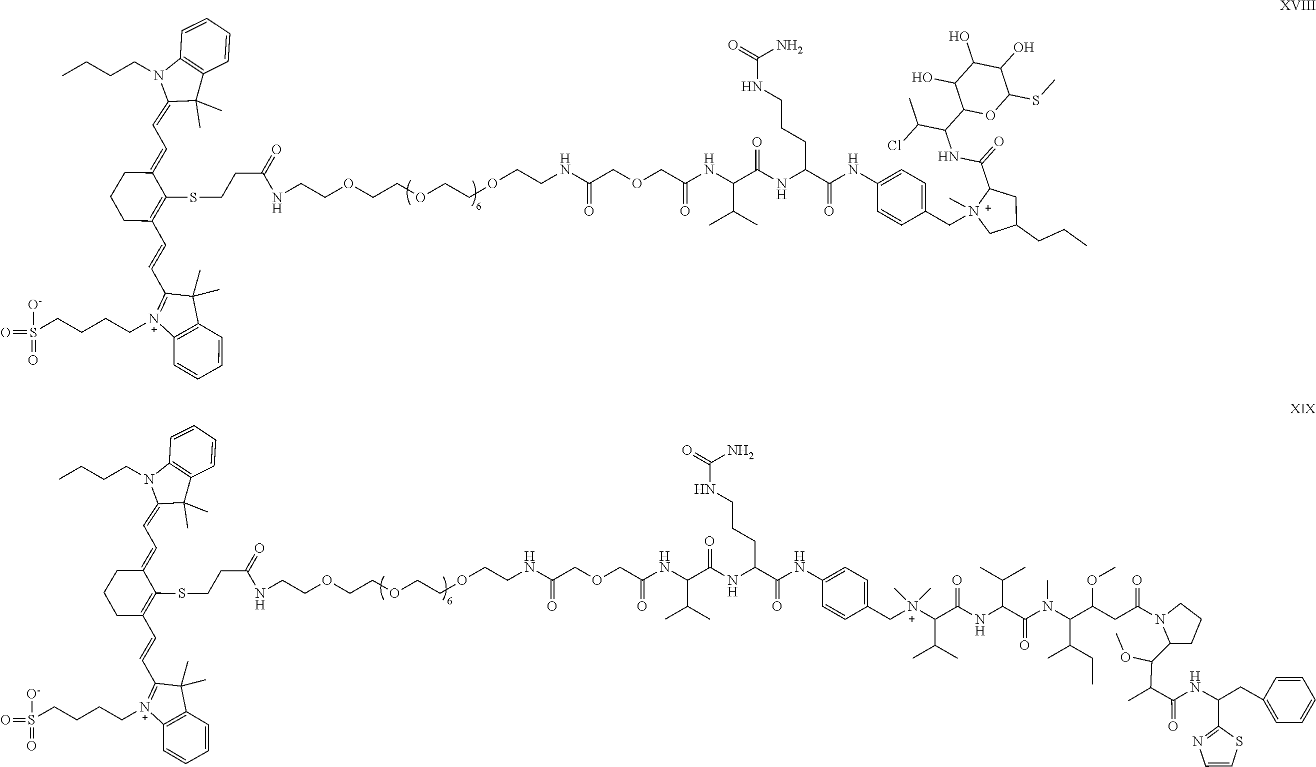

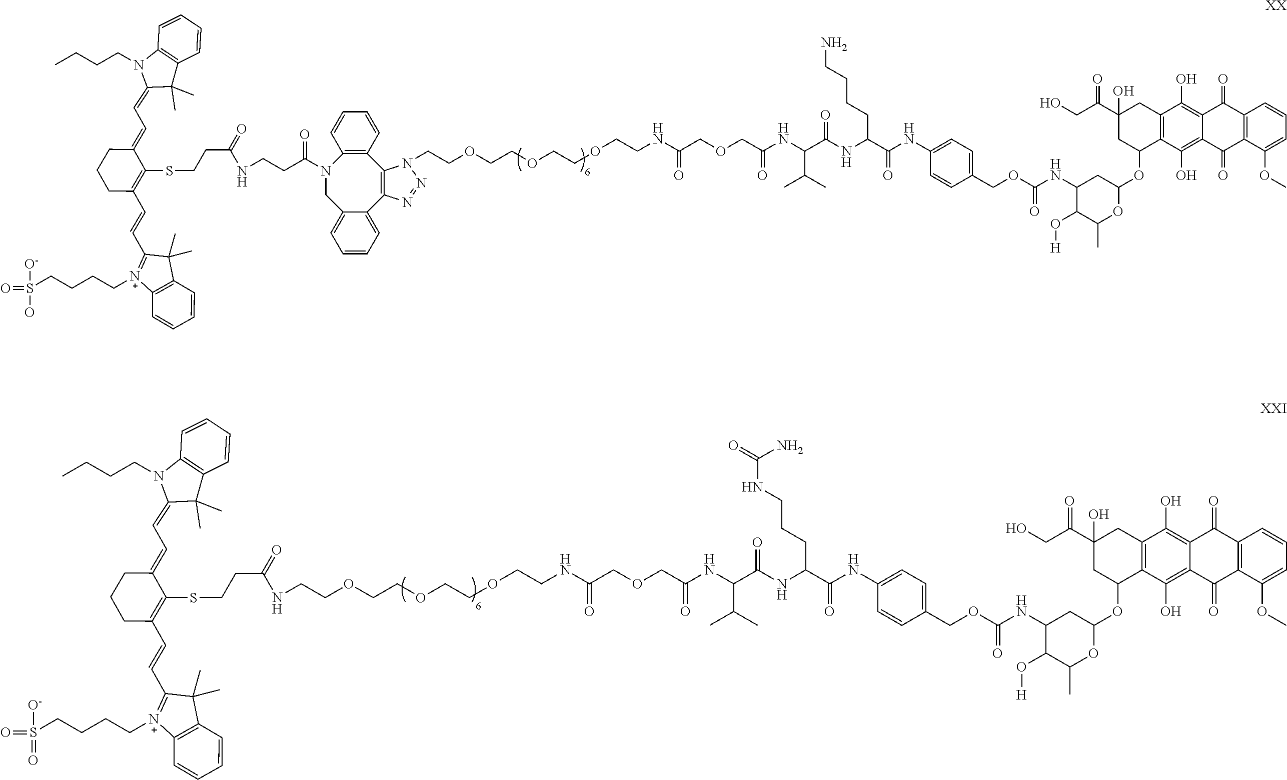

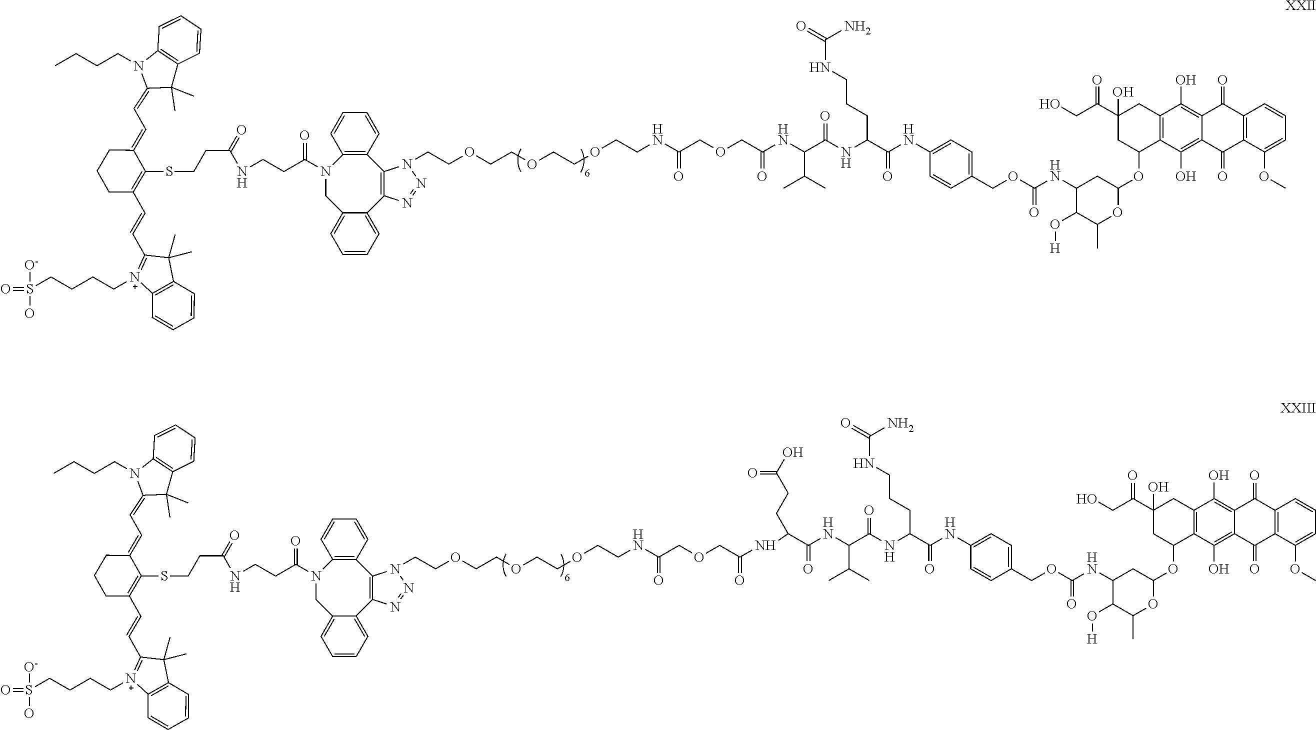

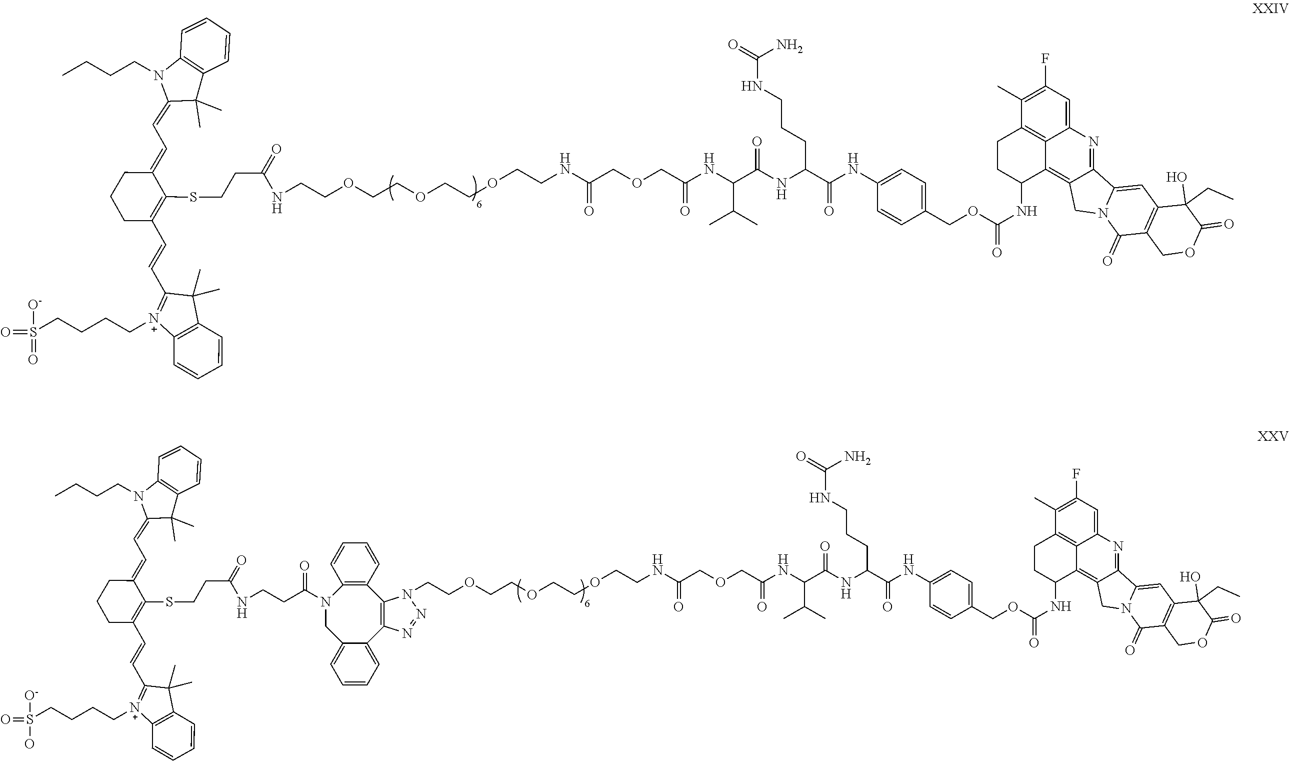

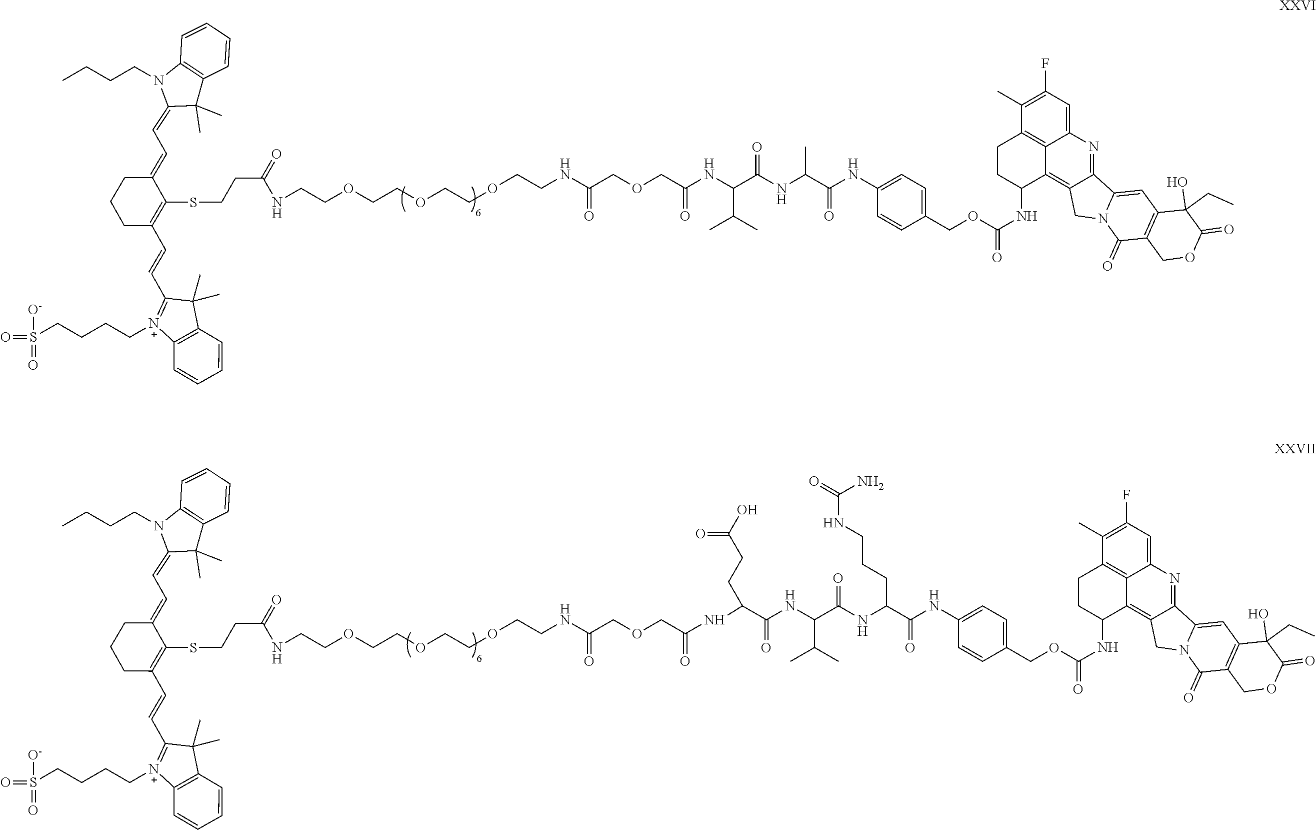

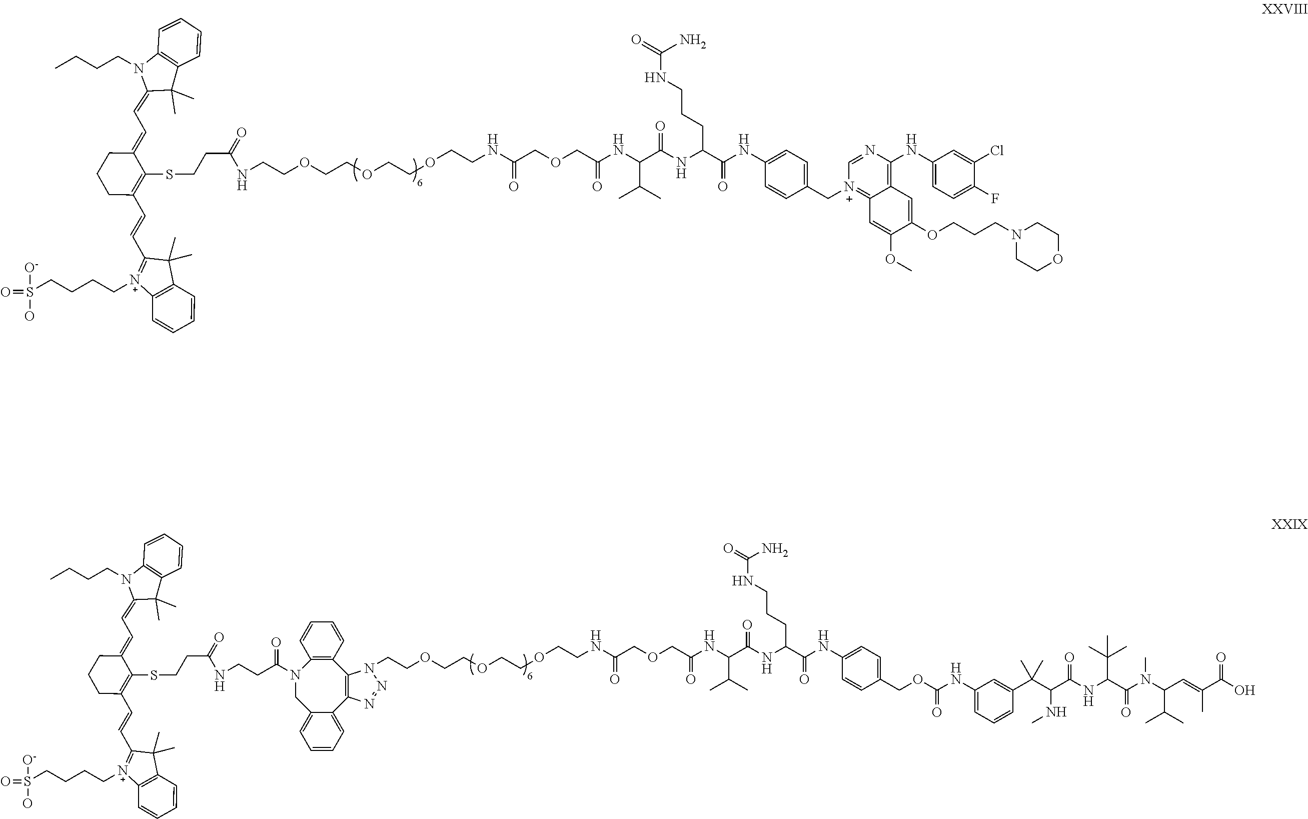

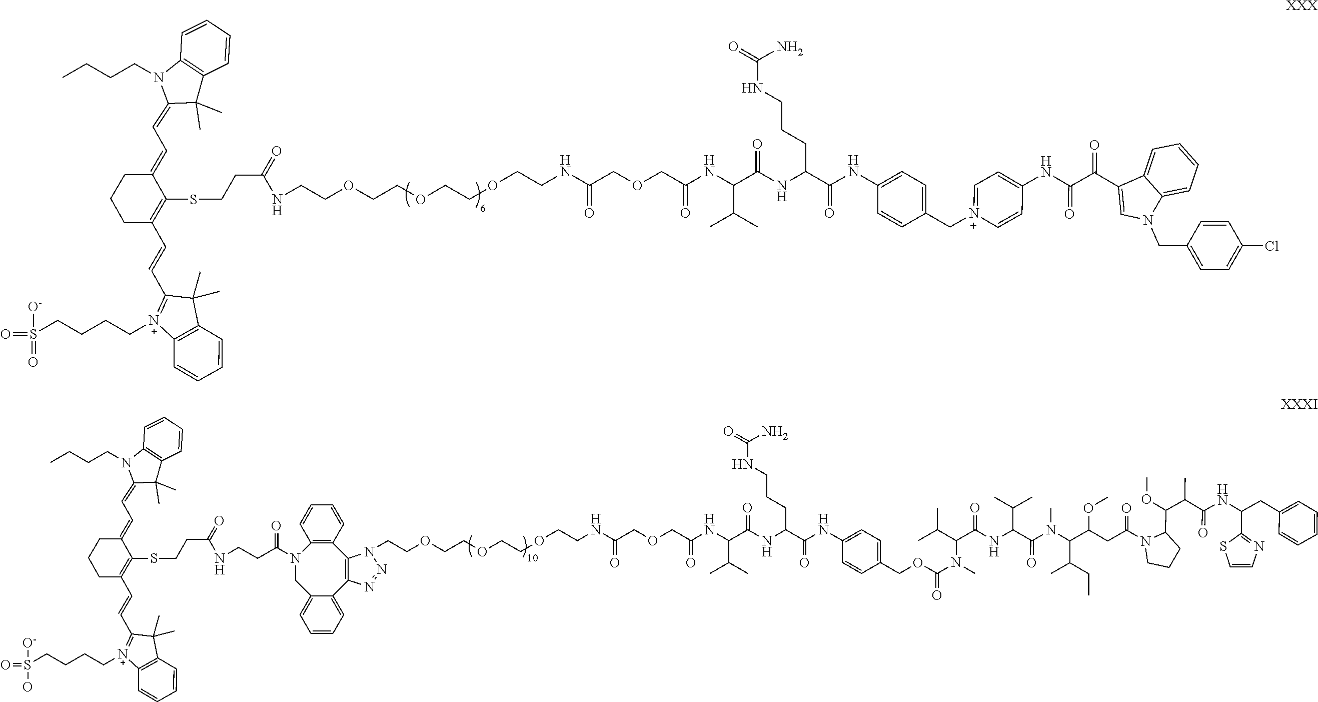

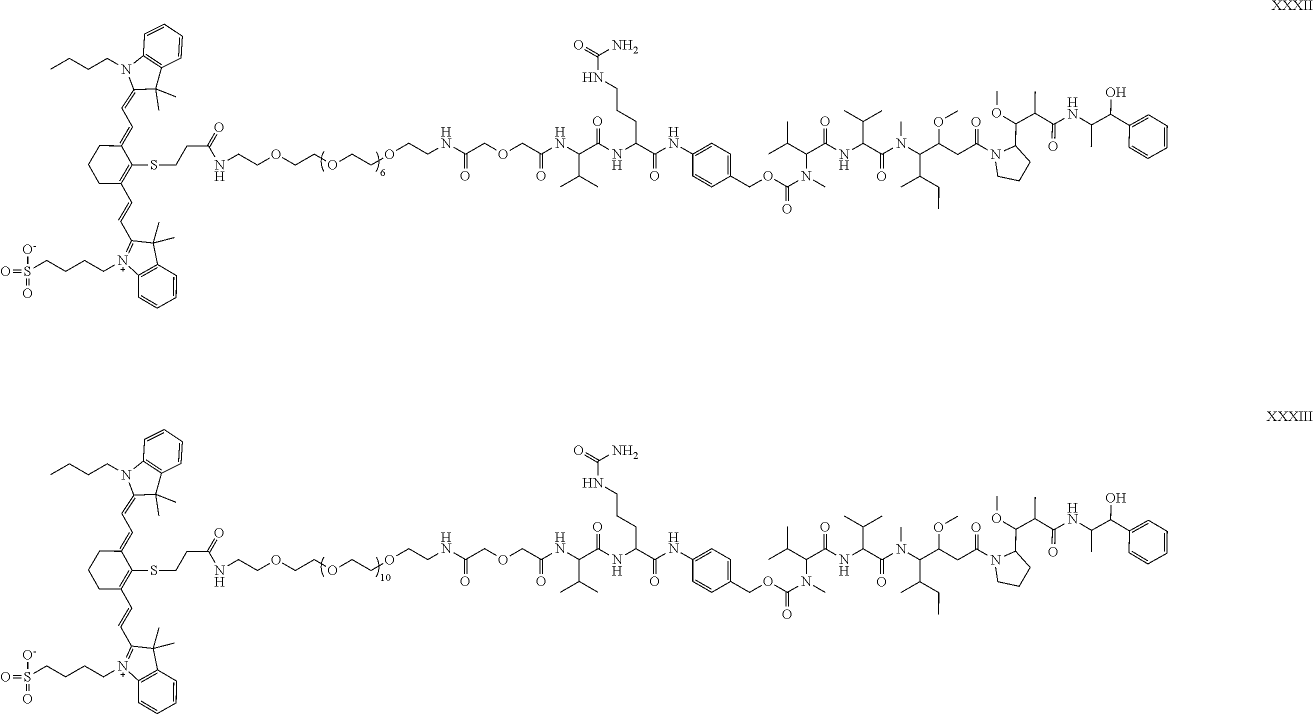

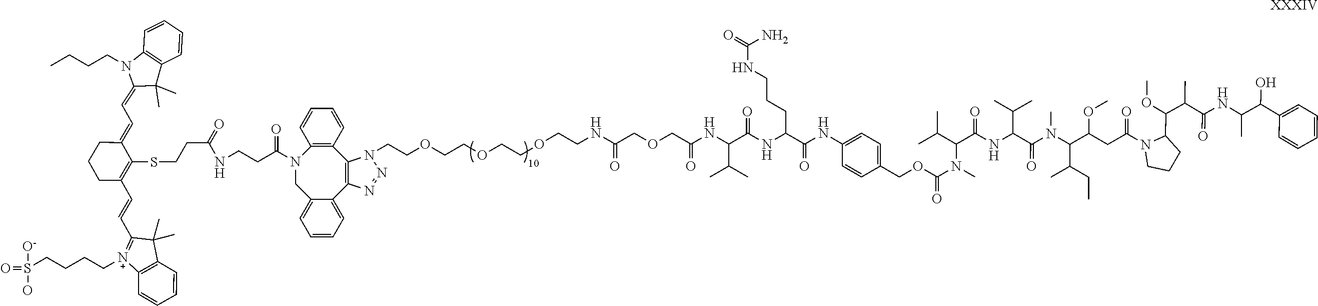

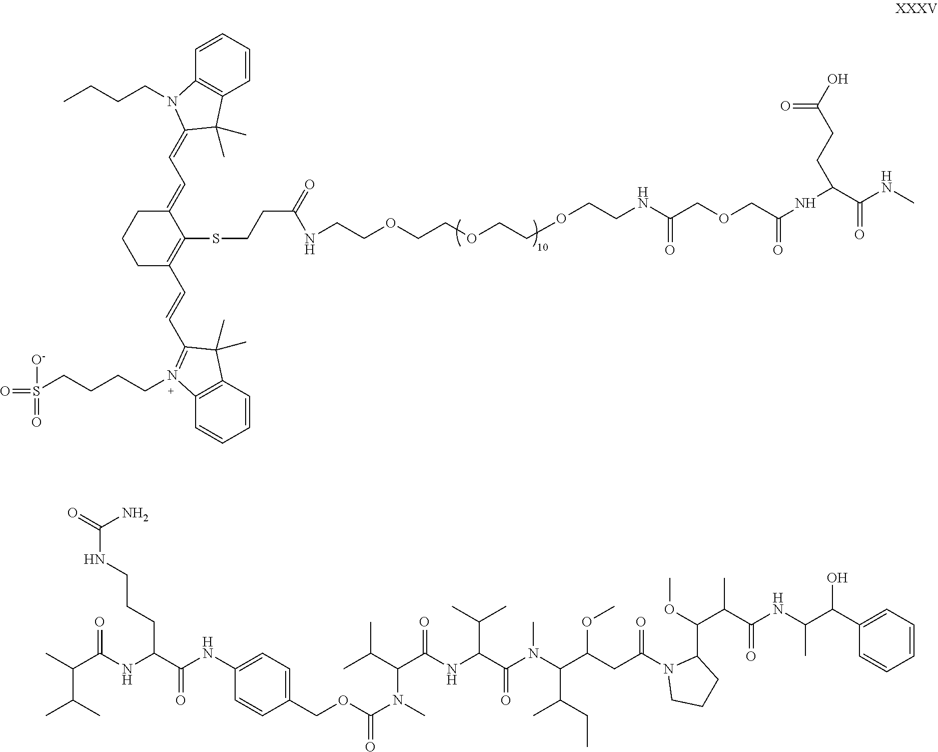

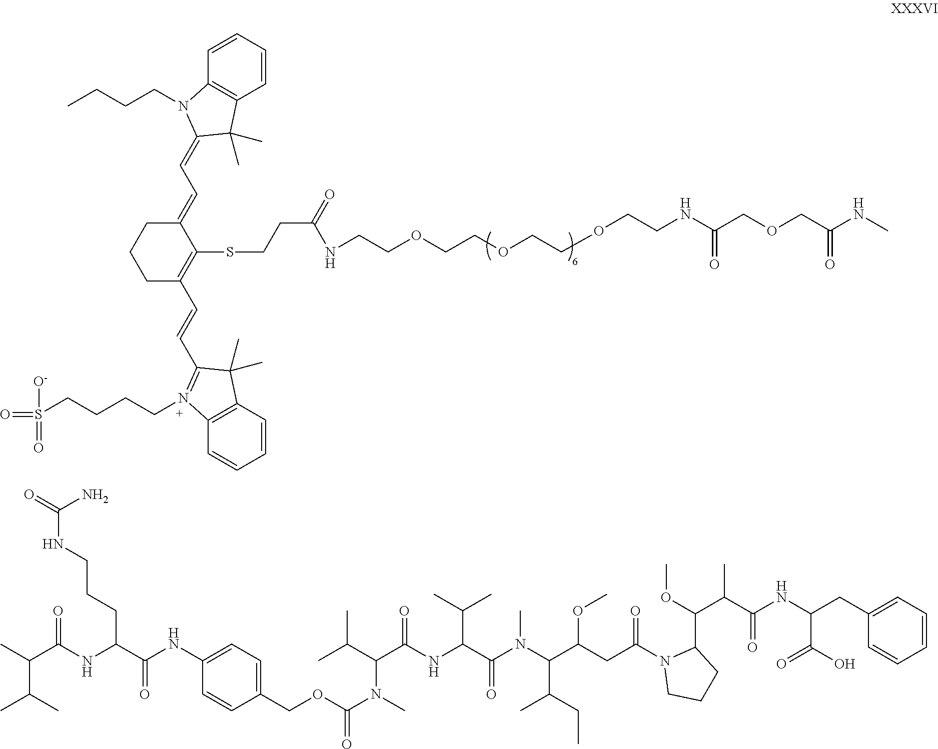

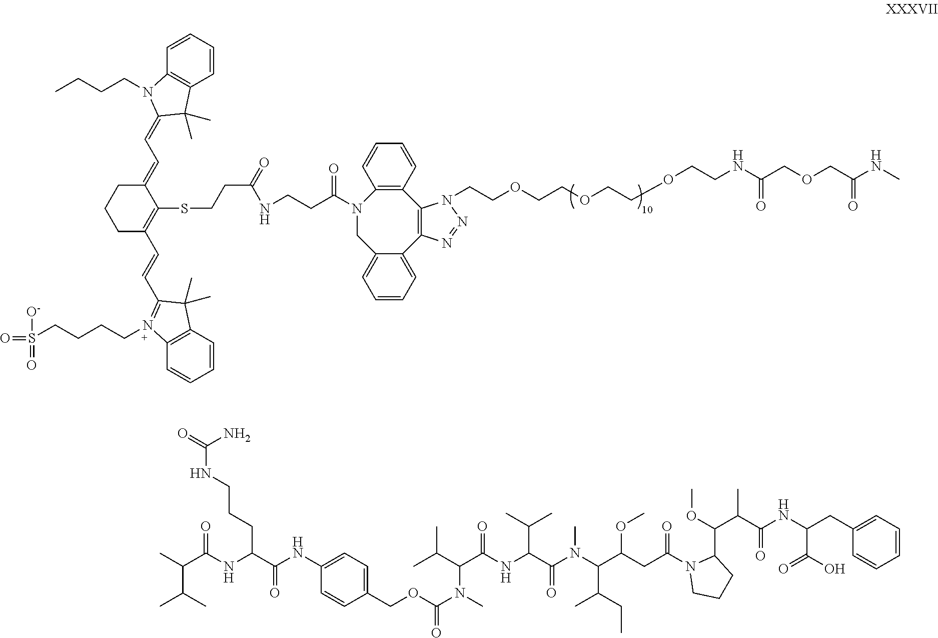

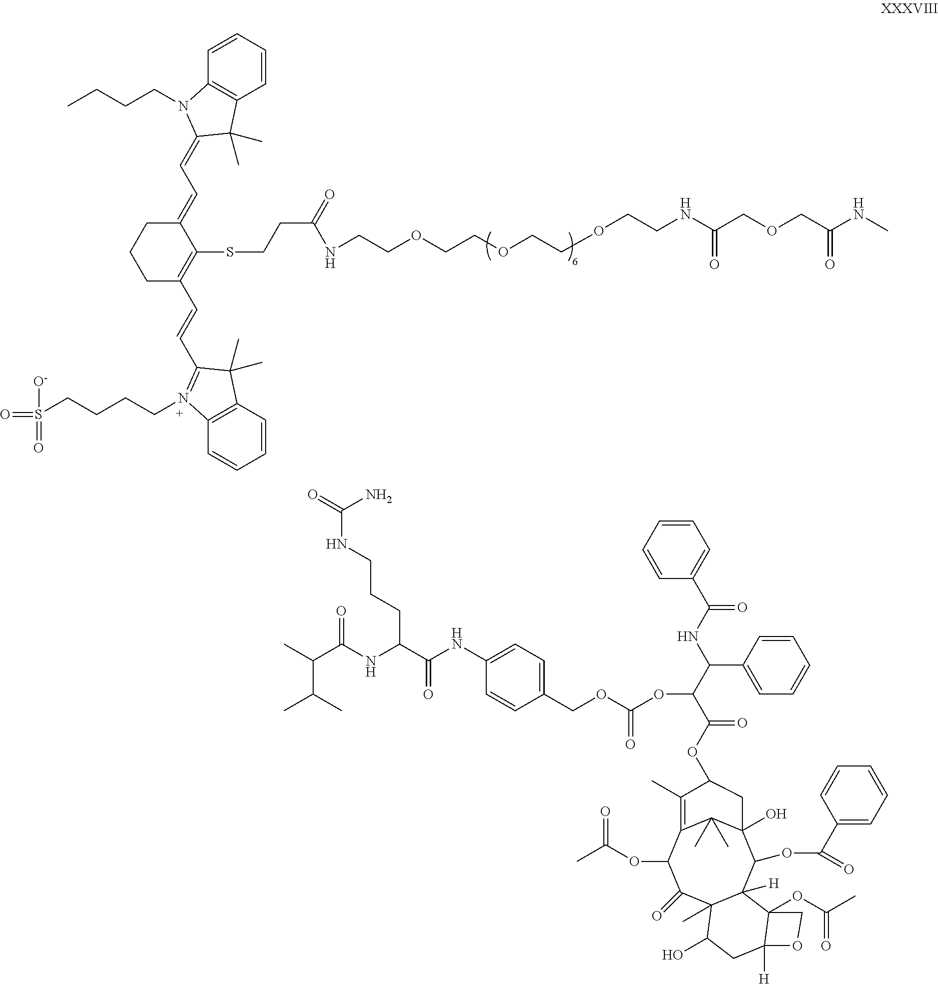

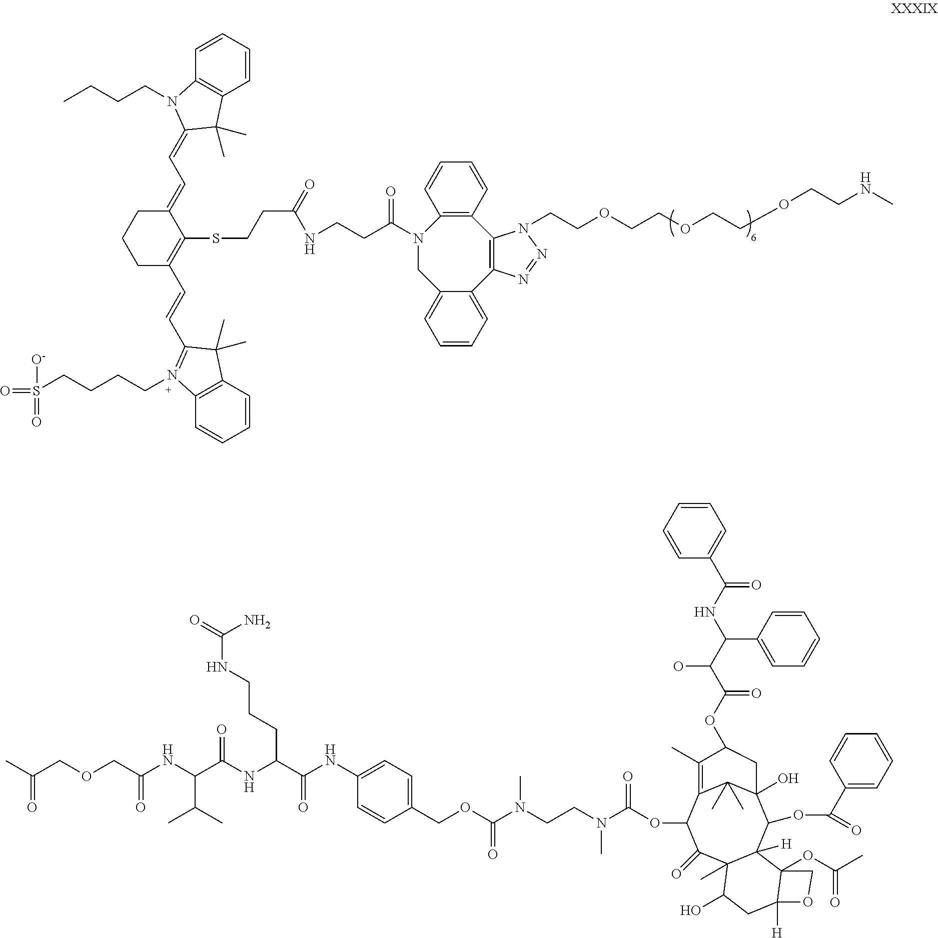

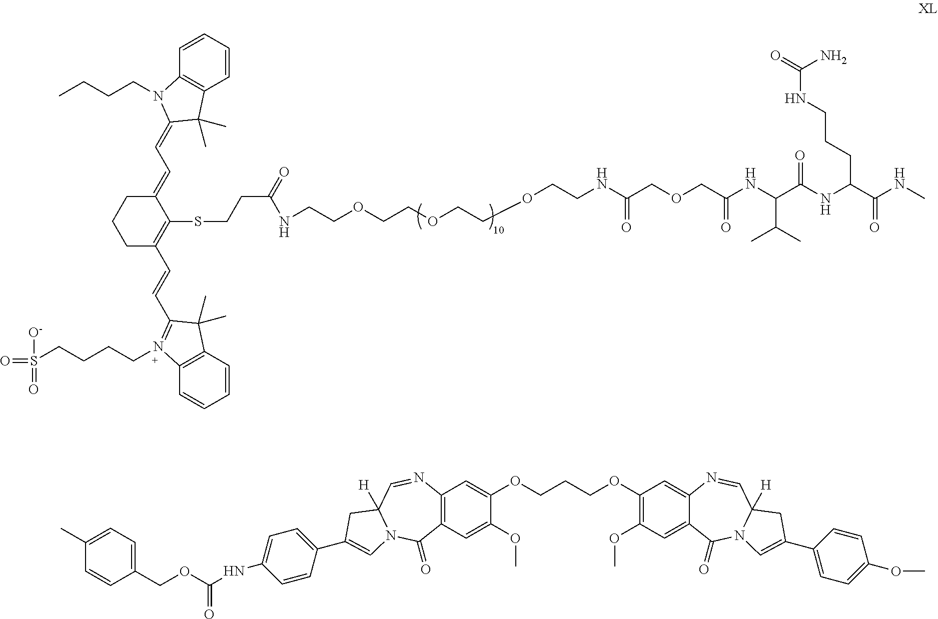

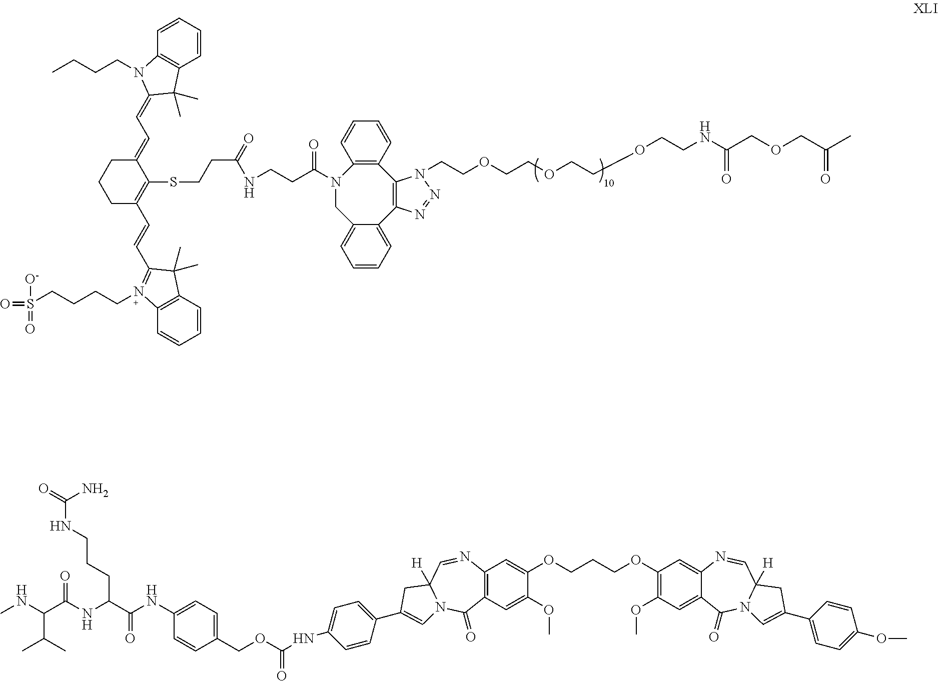

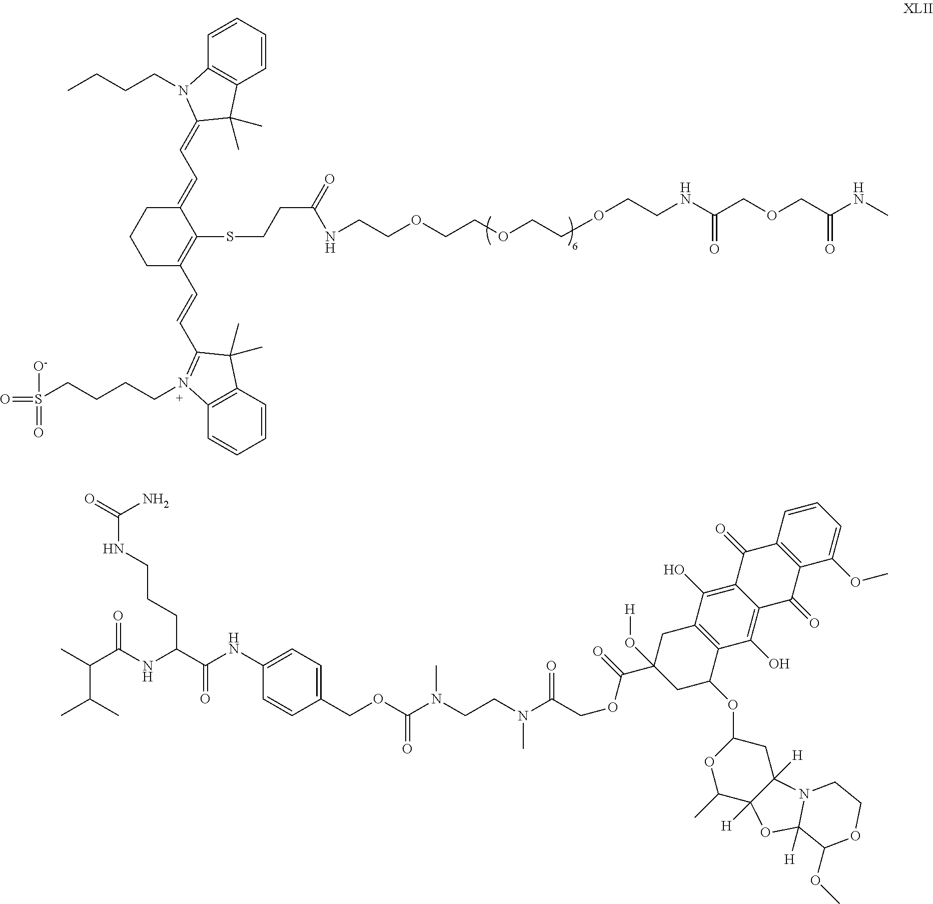

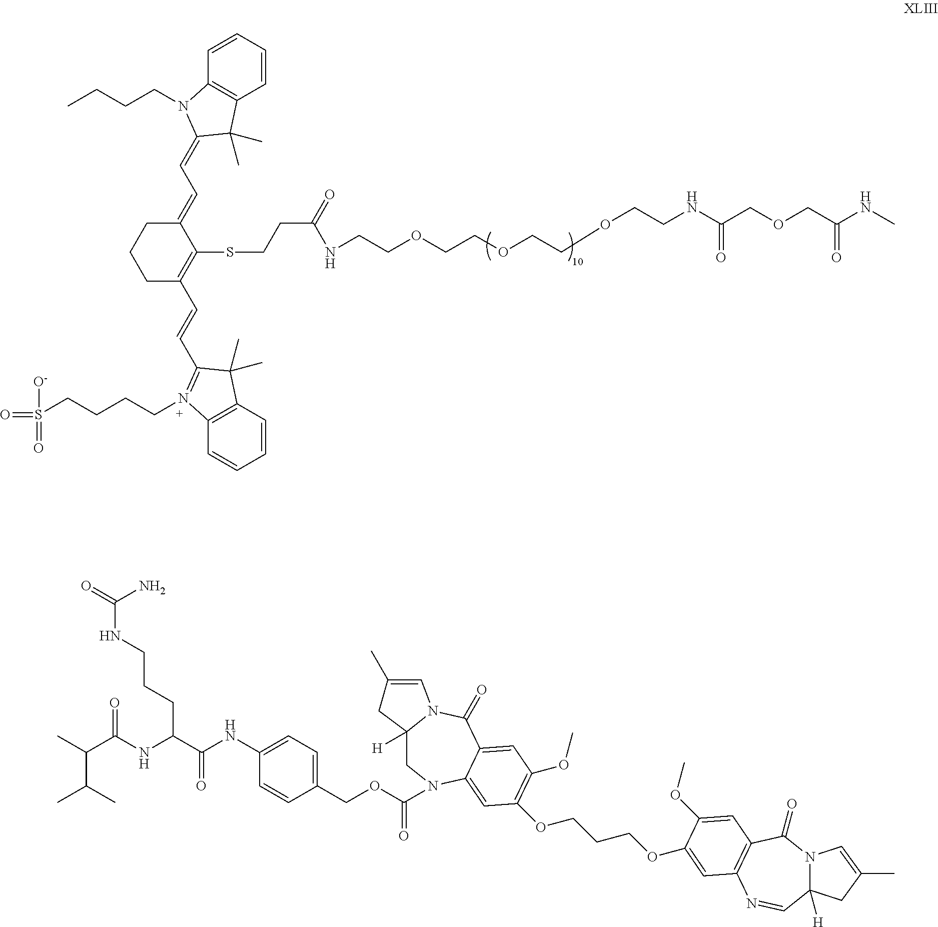

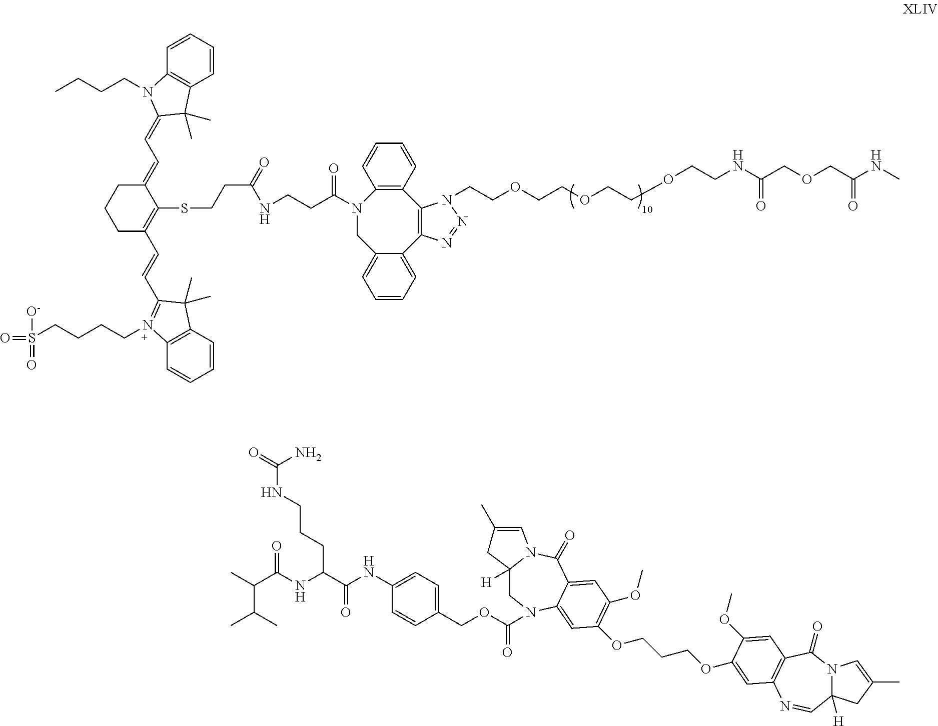

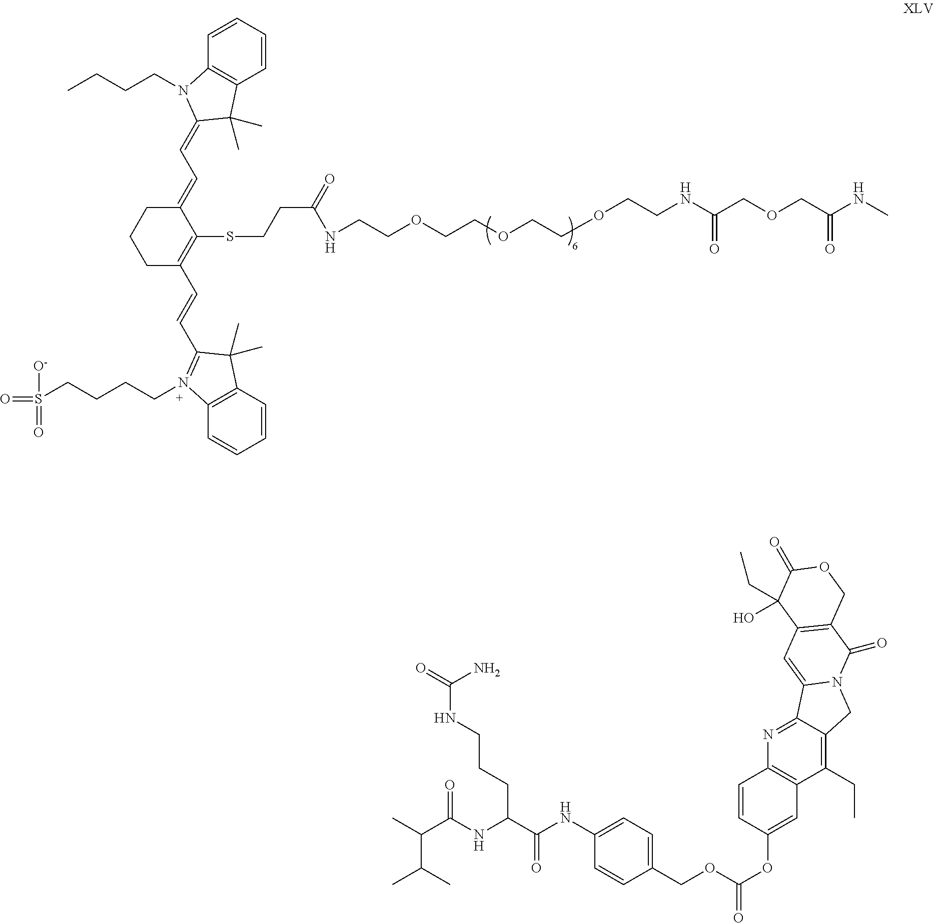

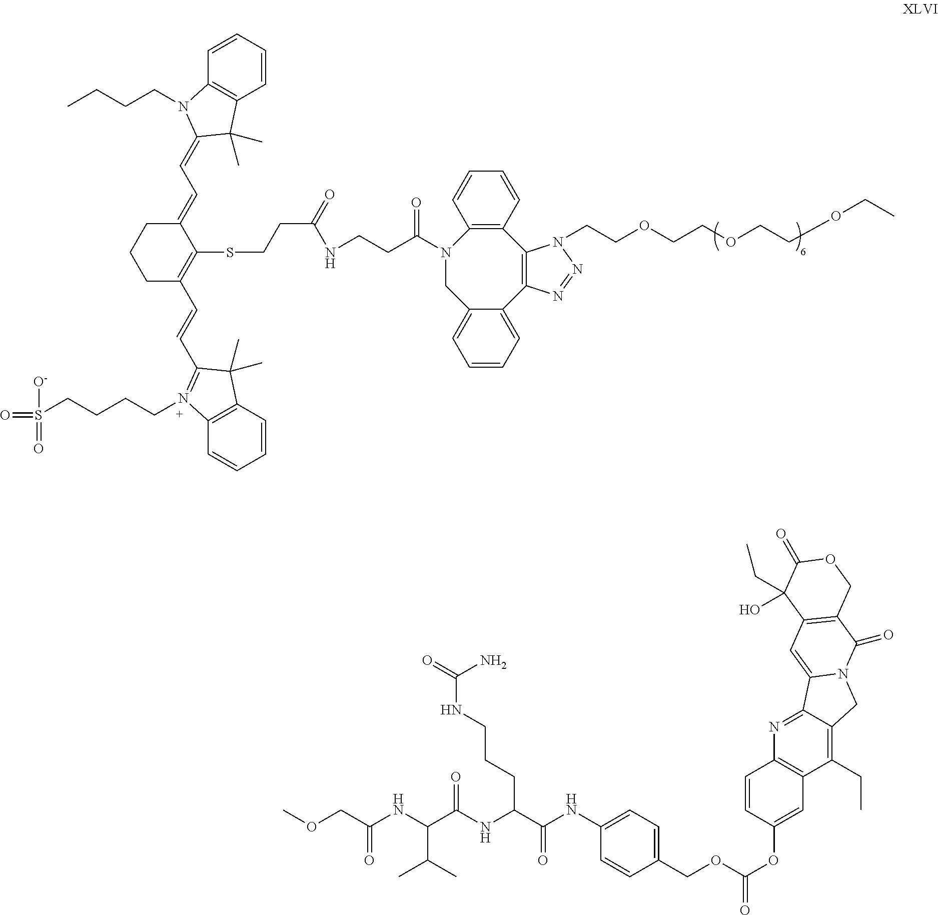

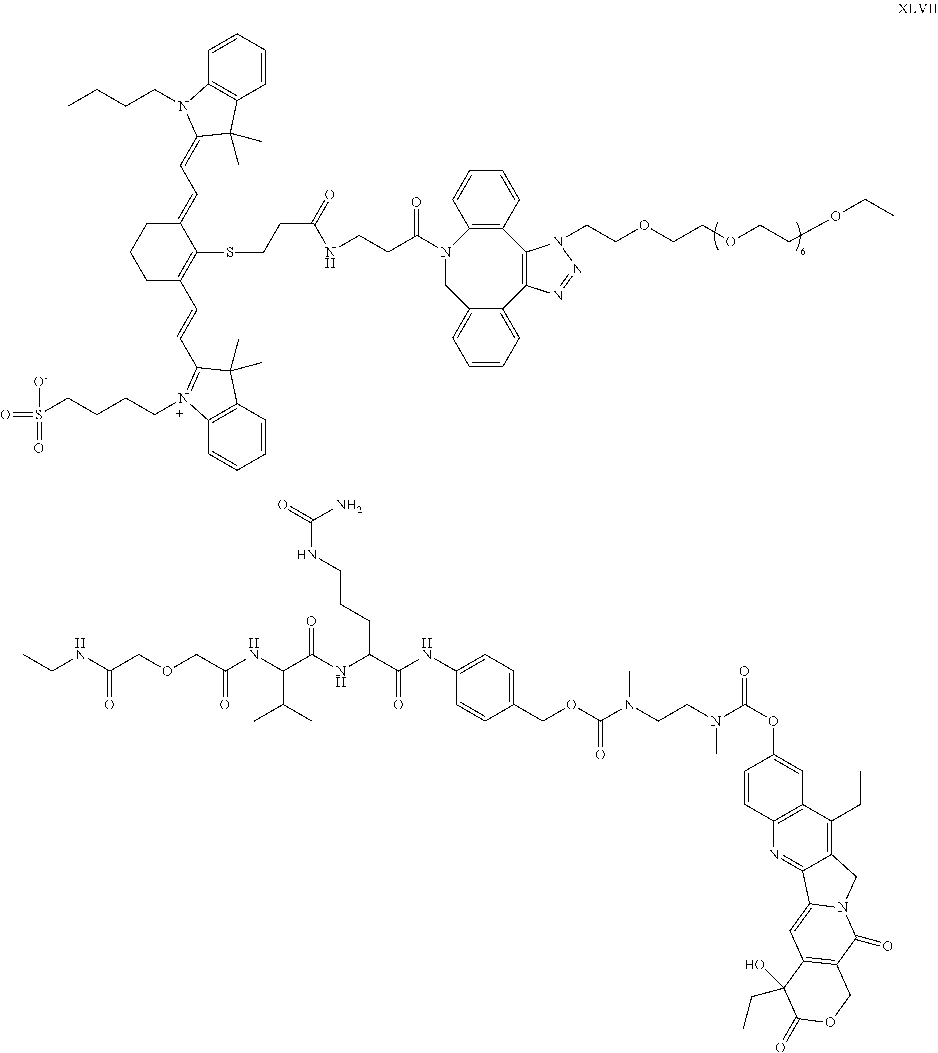

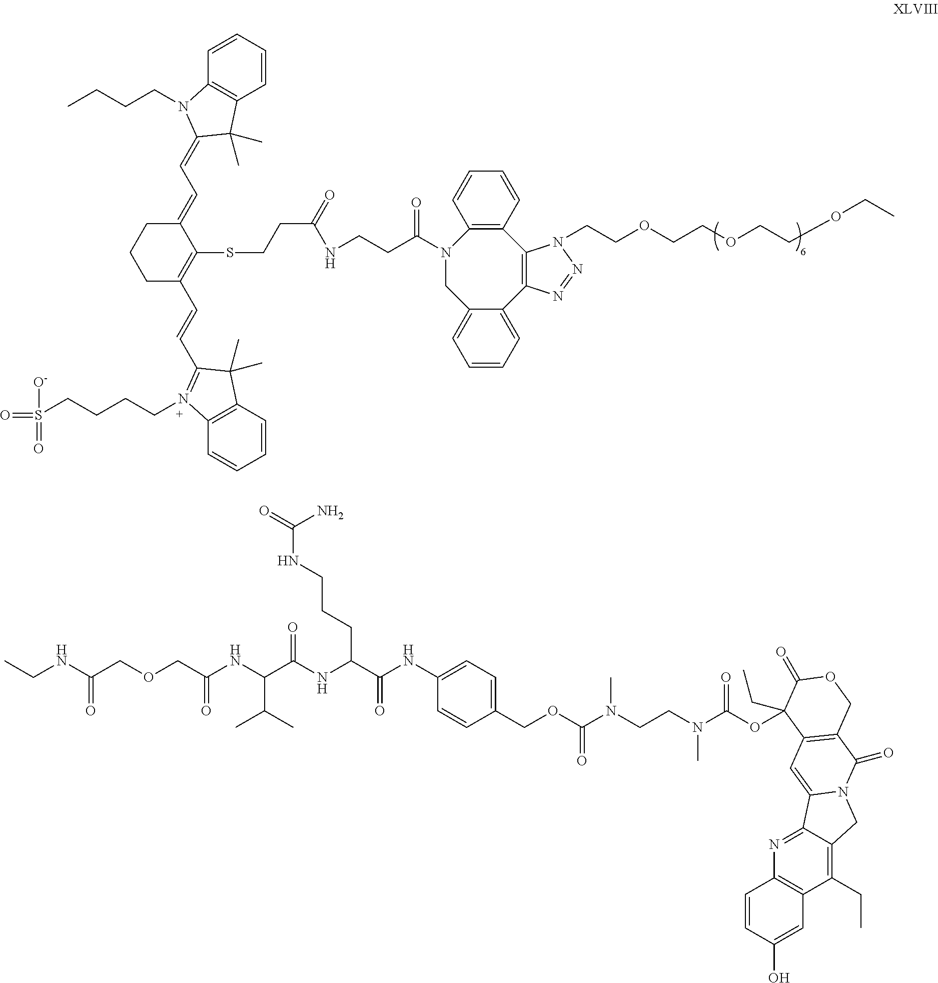

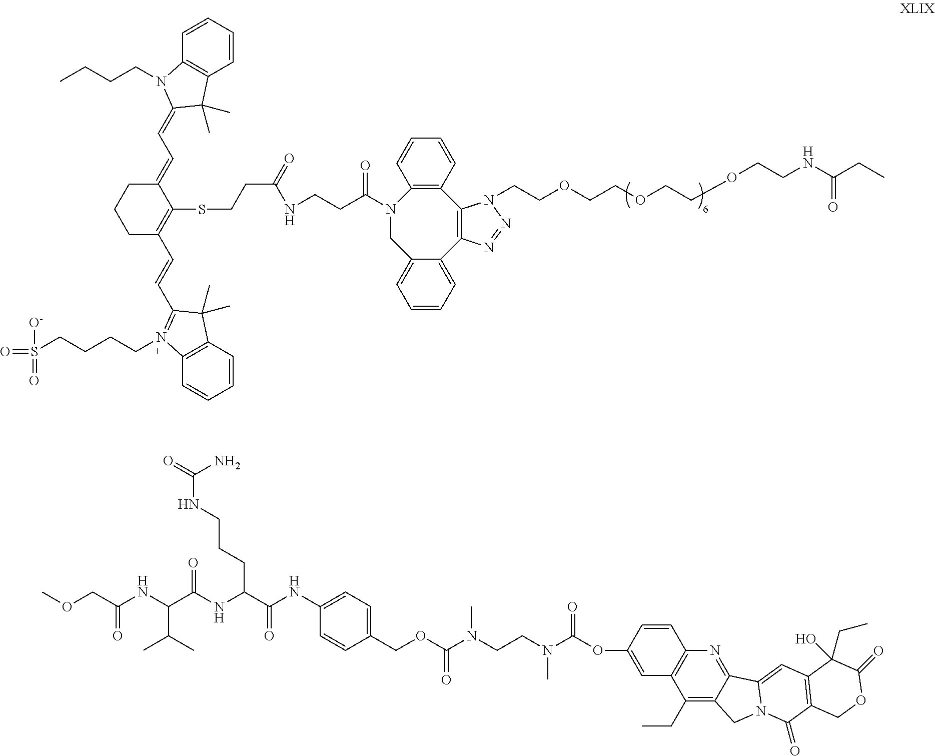

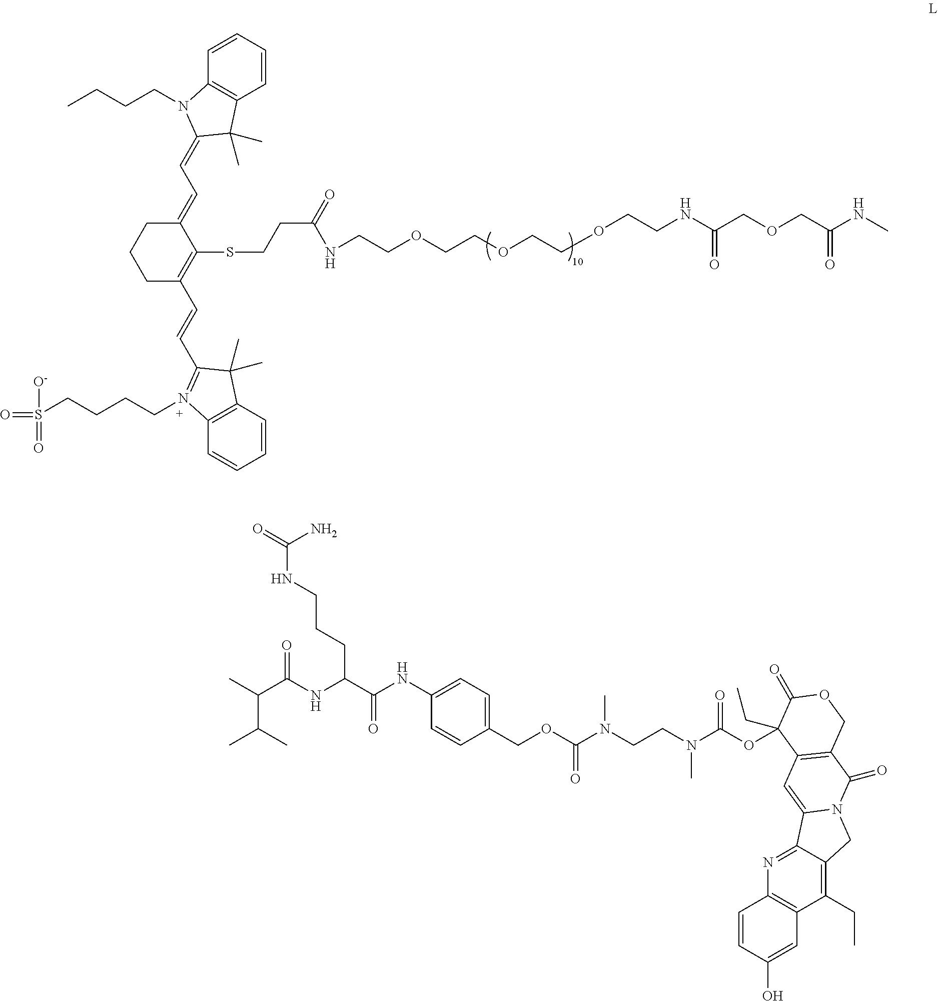

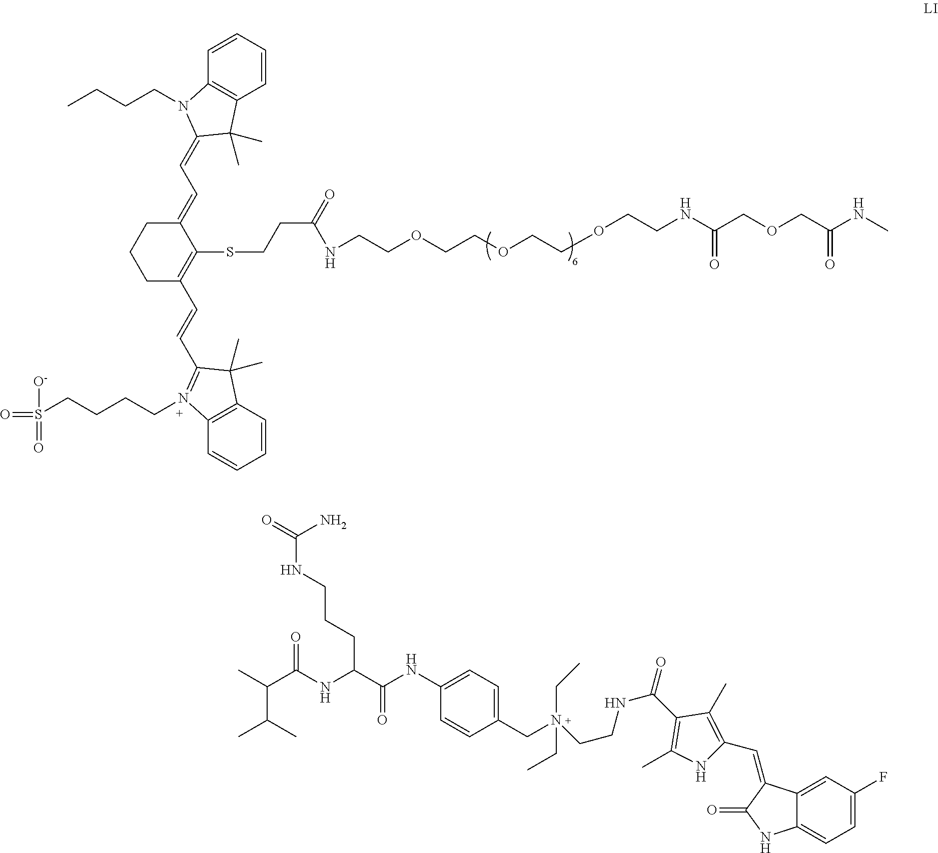

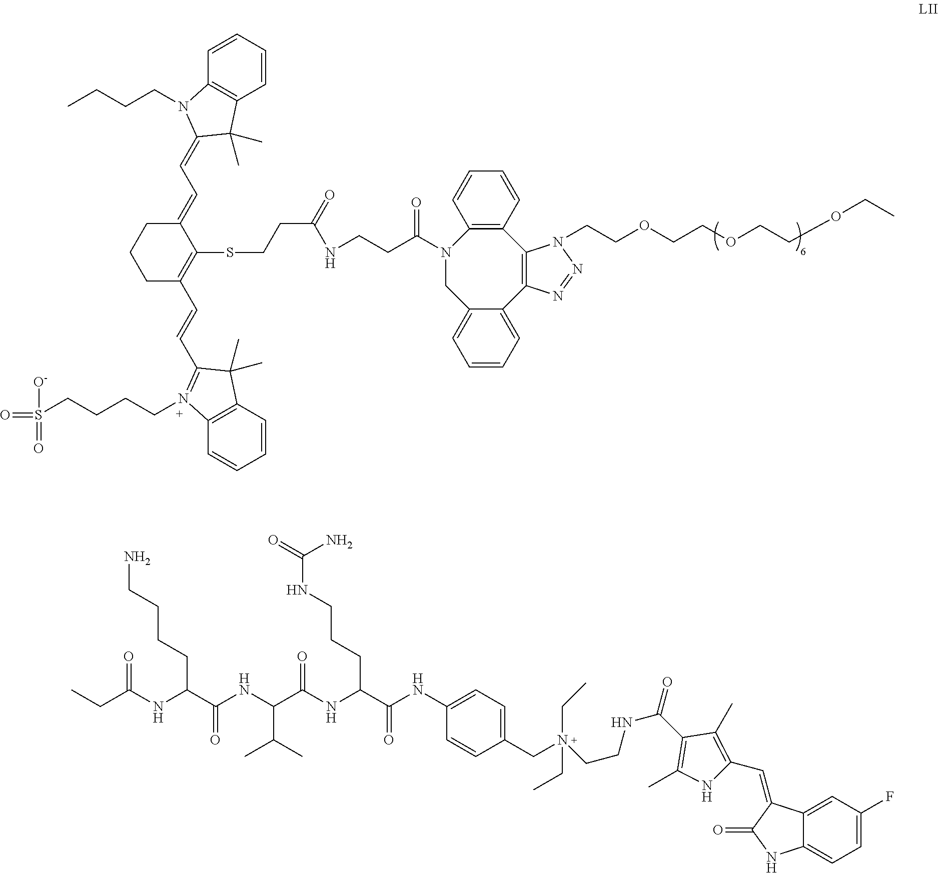

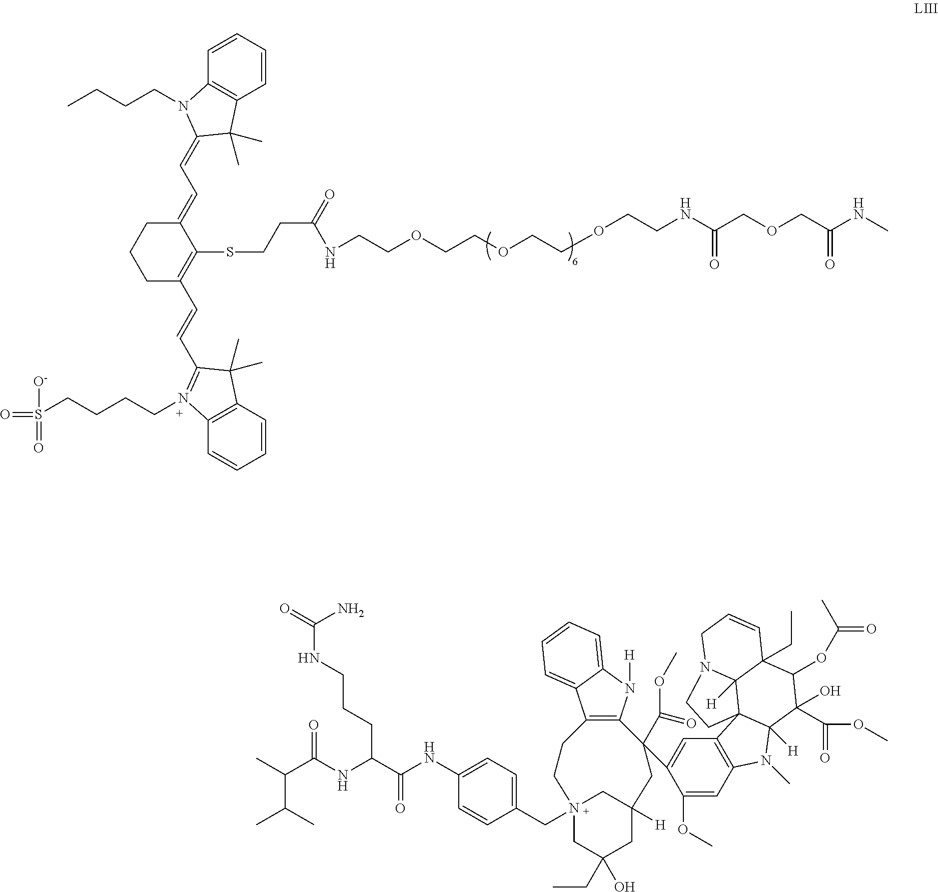

Exemplary conjugates comprising a tumor-targeting NIR dye and specifically, the dye shown in Formula VII, coupled to a chemotherapeutic agent via a linker including a cleavable peptide portion which may be useful with the present invention are shown in Formulas LIV through LXIX.

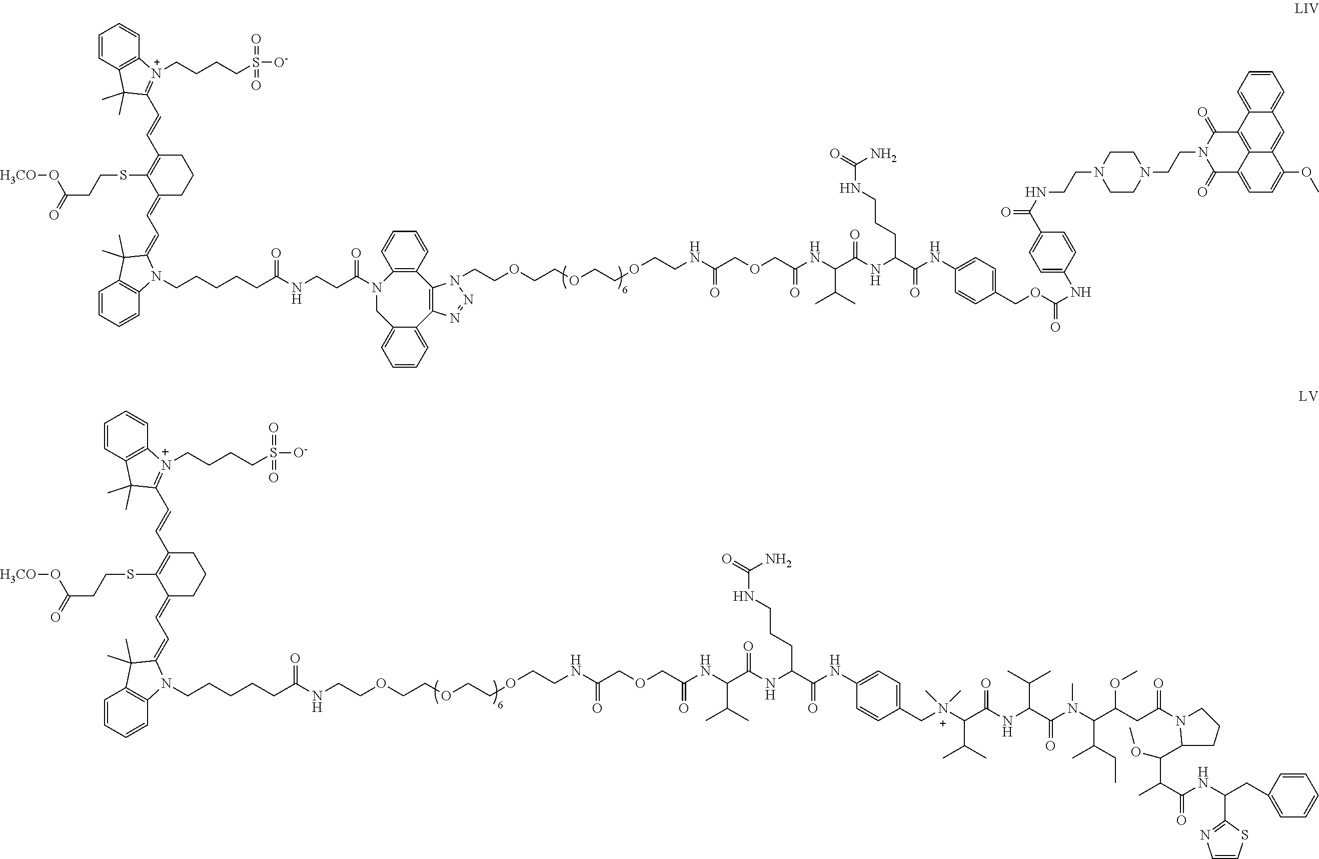

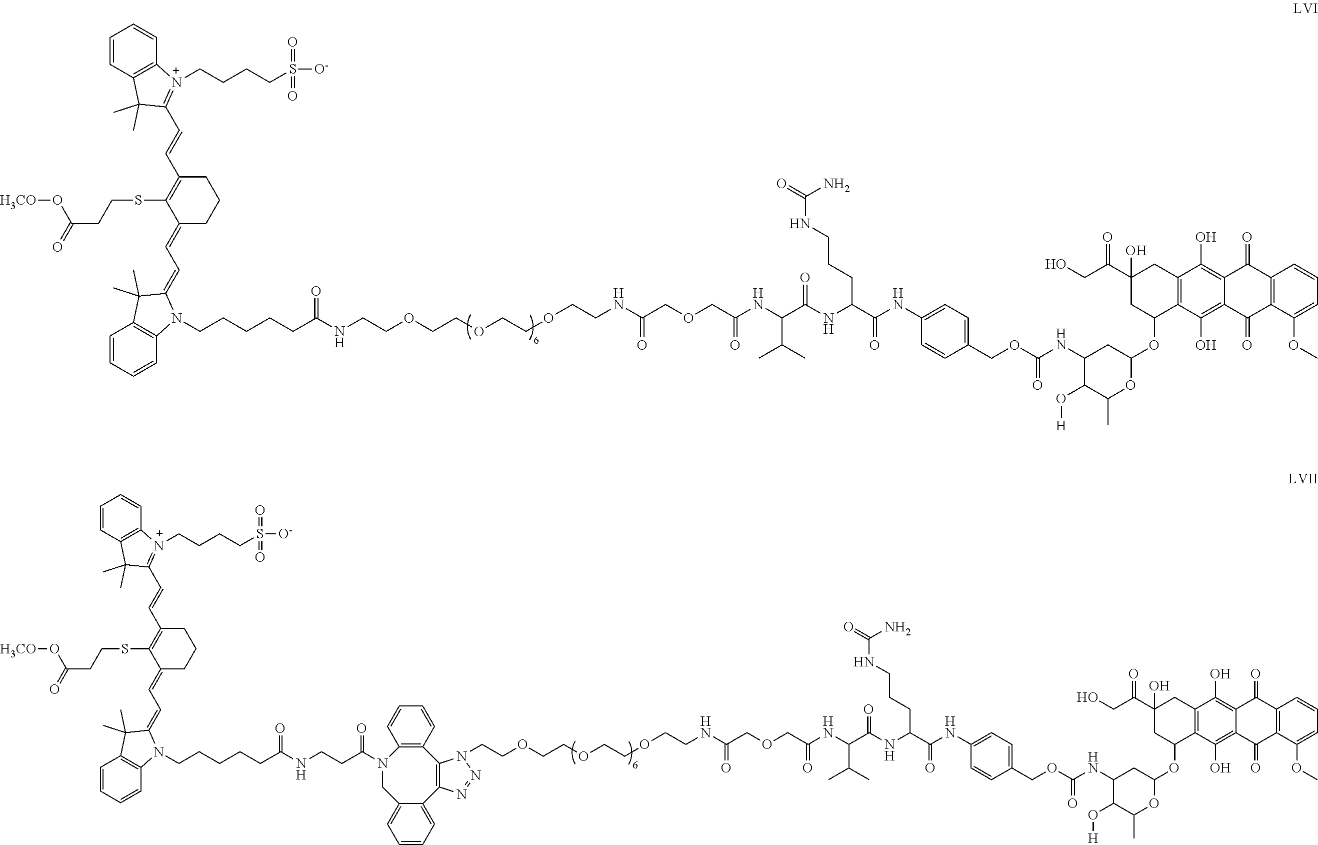

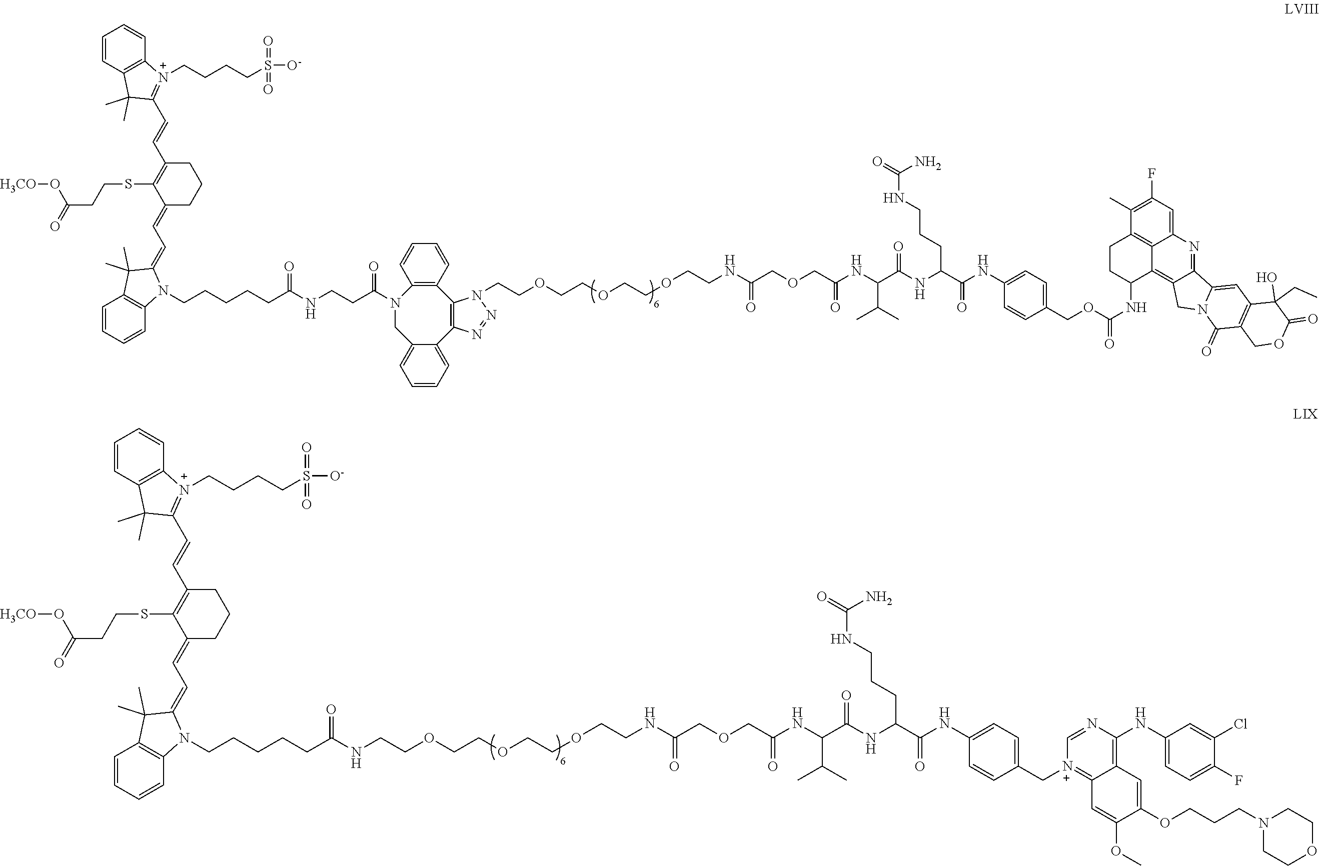

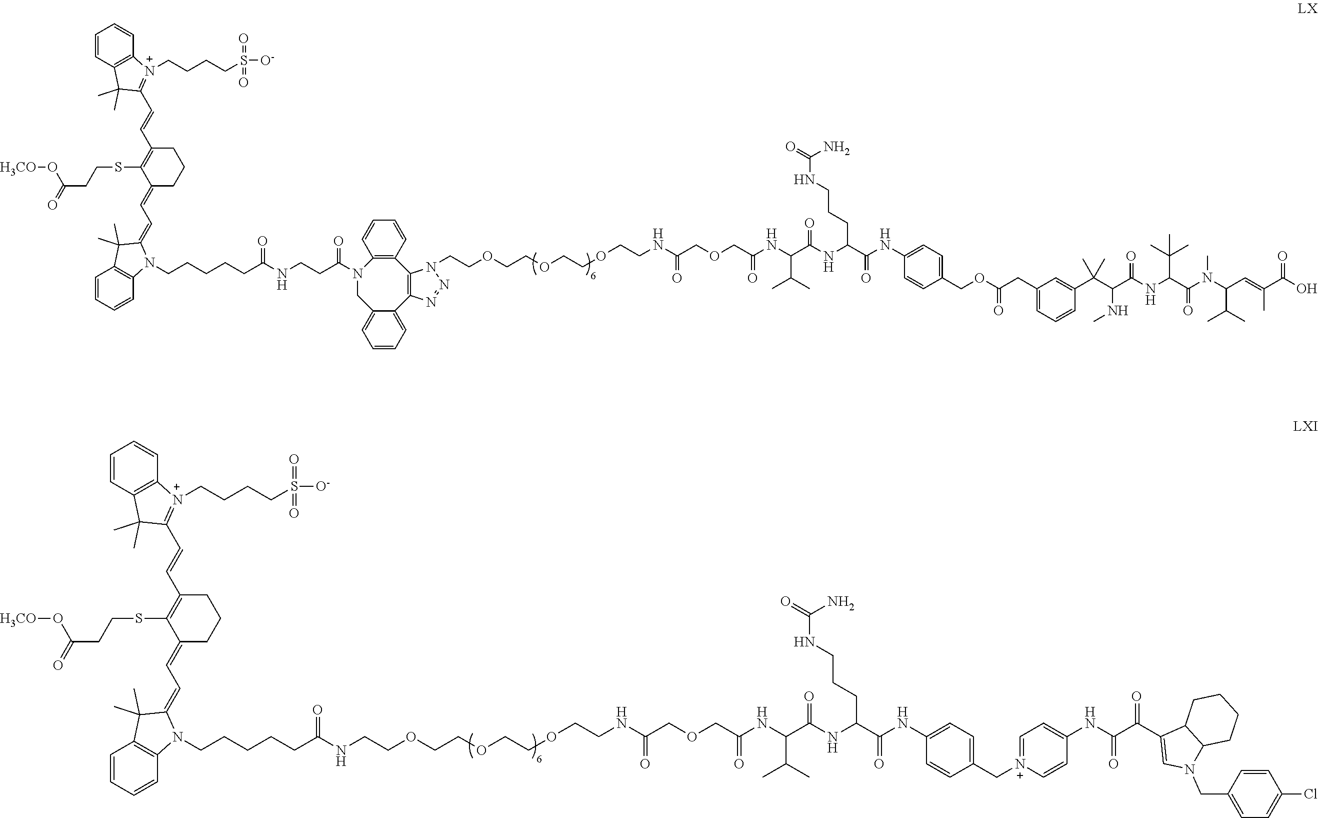

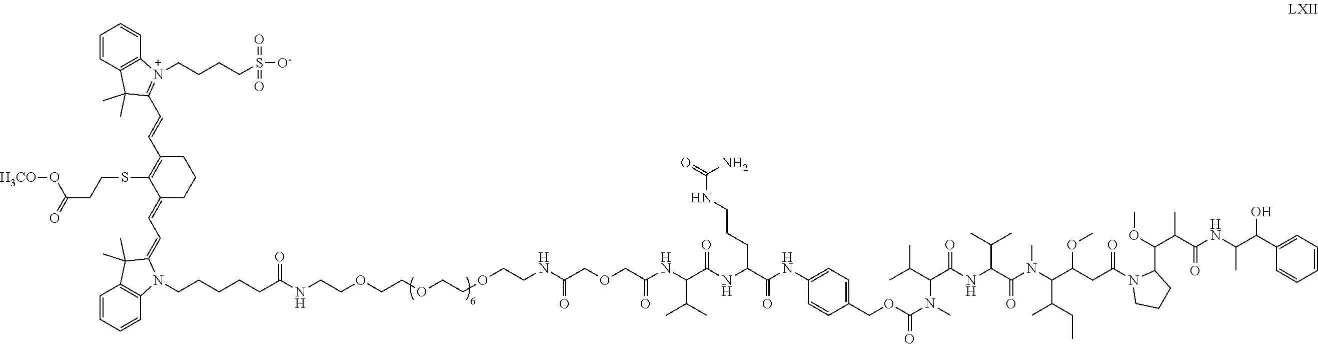

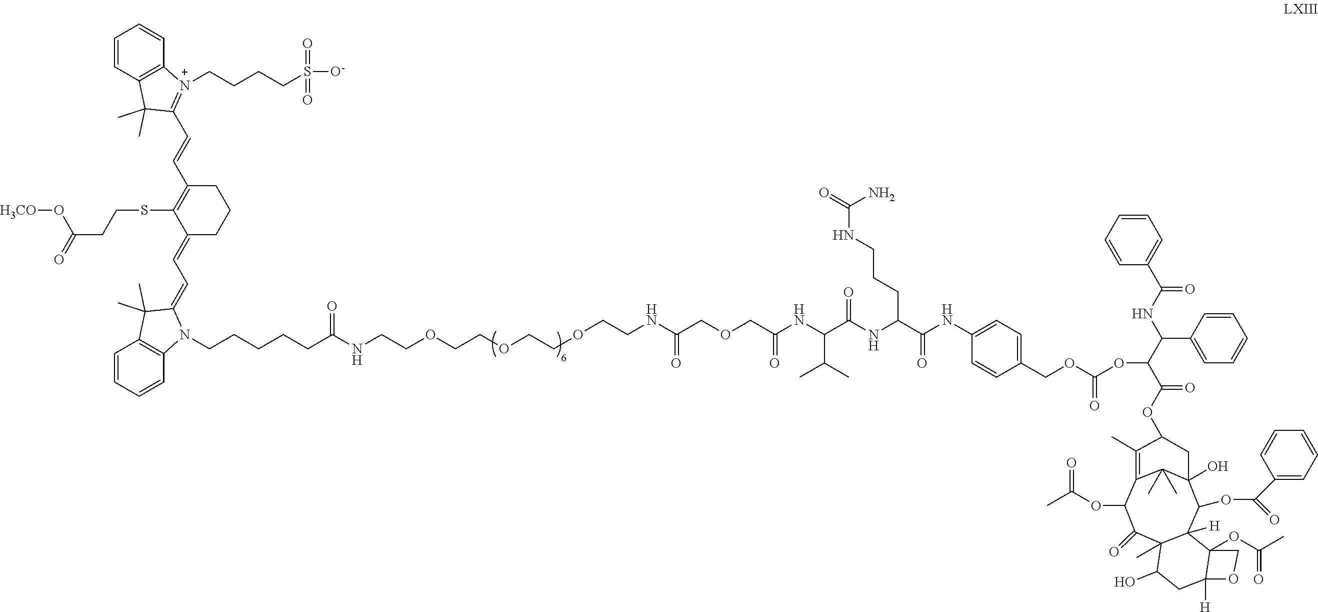

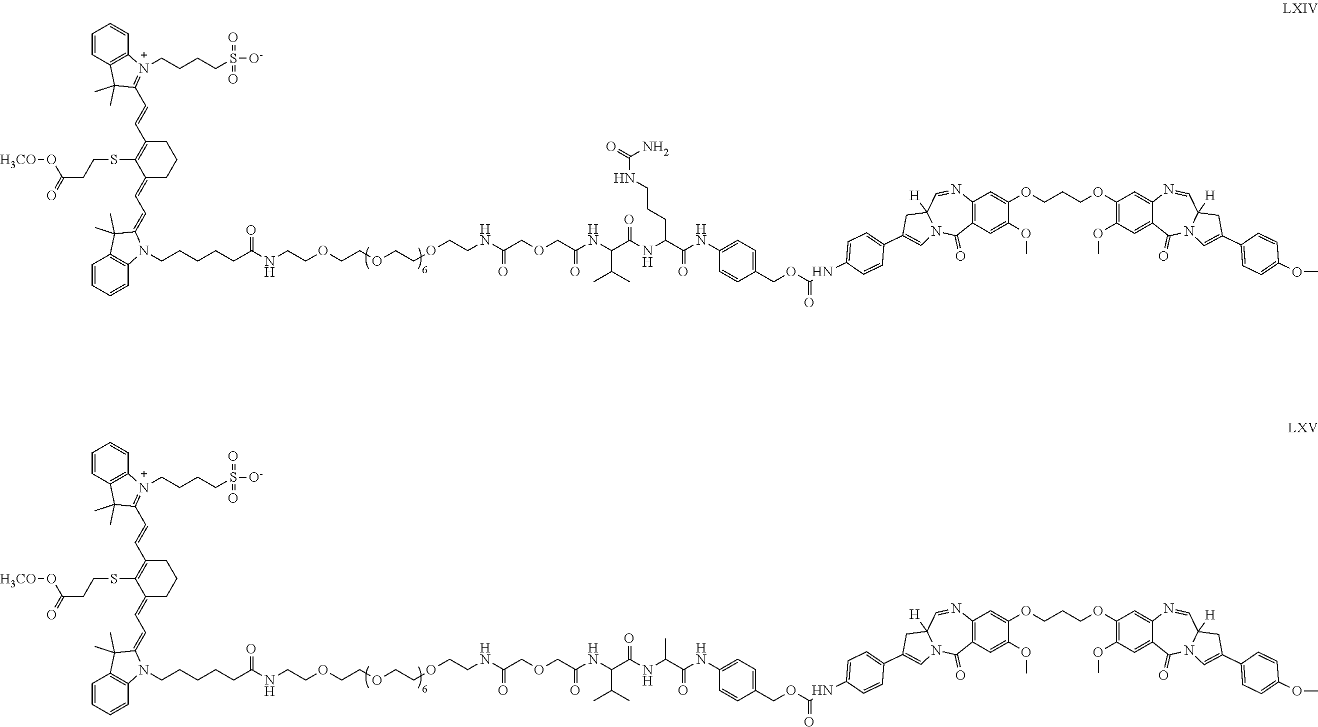

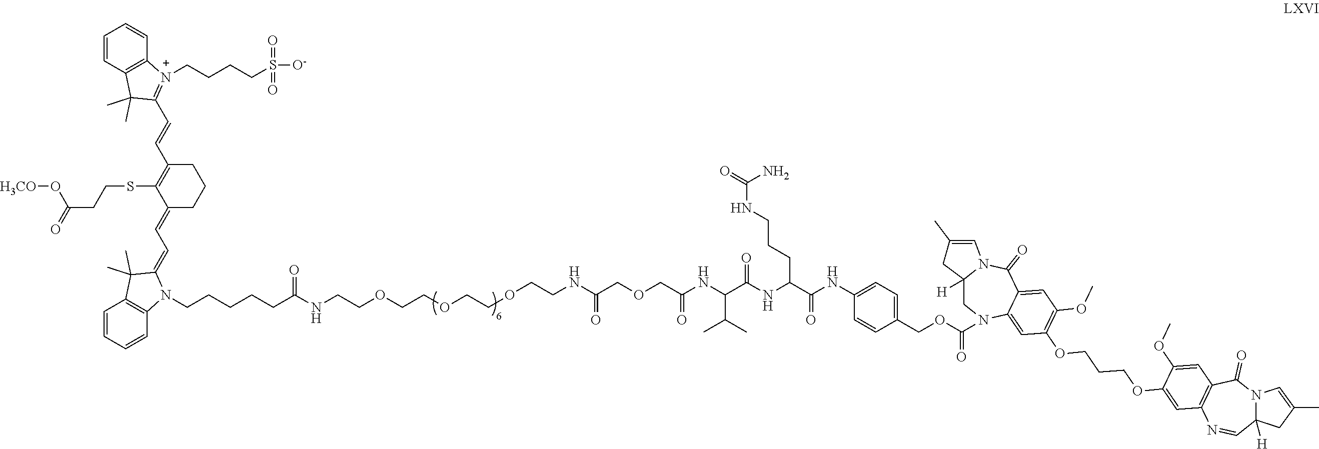

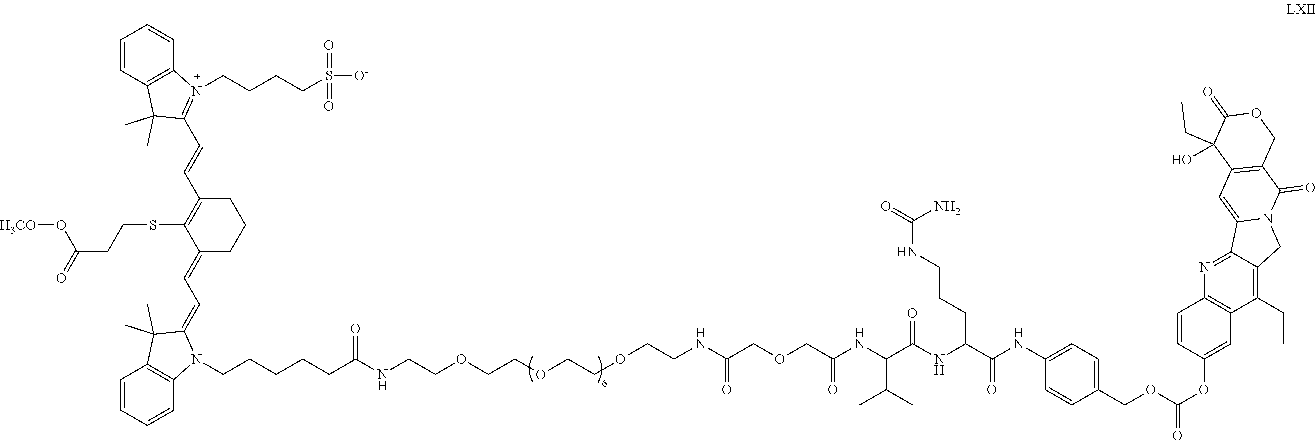

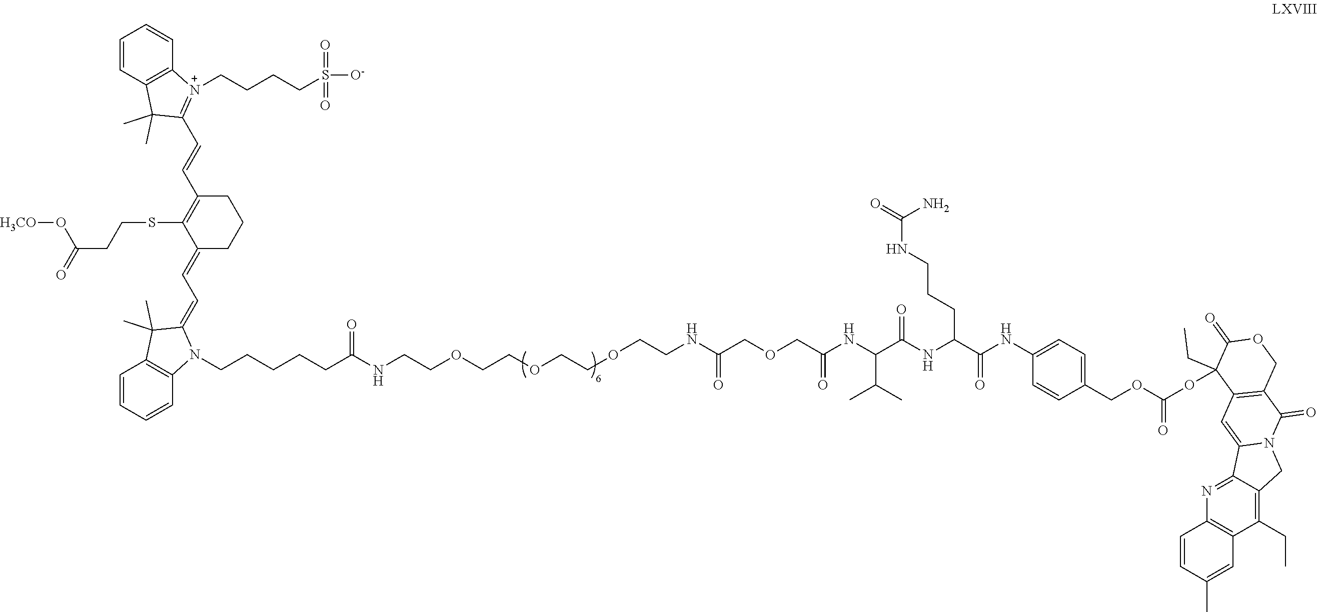

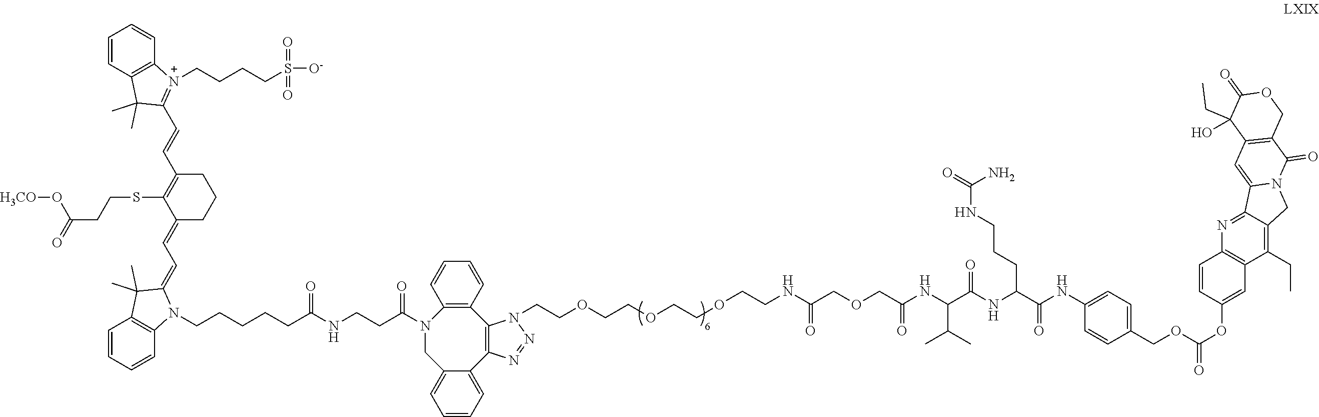

Exemplary conjugates comprising a tumor-targeting NIR dye and specifically, the dye shown in Formula VIII, coupled to a chemotherapeutic agent via a linker including a cleavable peptide portion which may be useful with the present invention are shown in Formulas LXX through LXXIV.

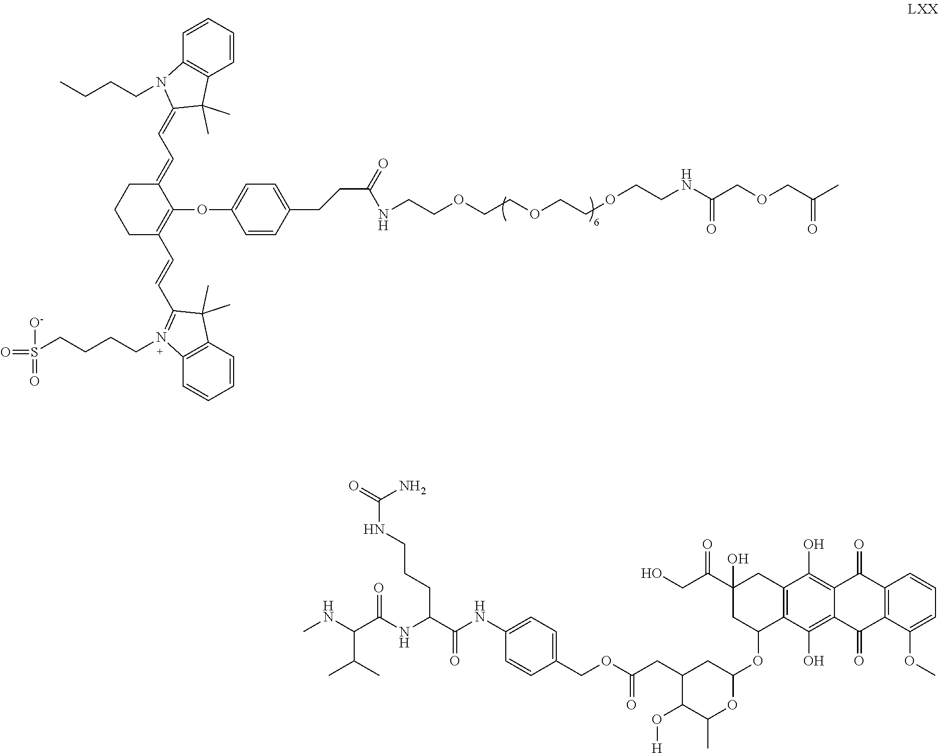

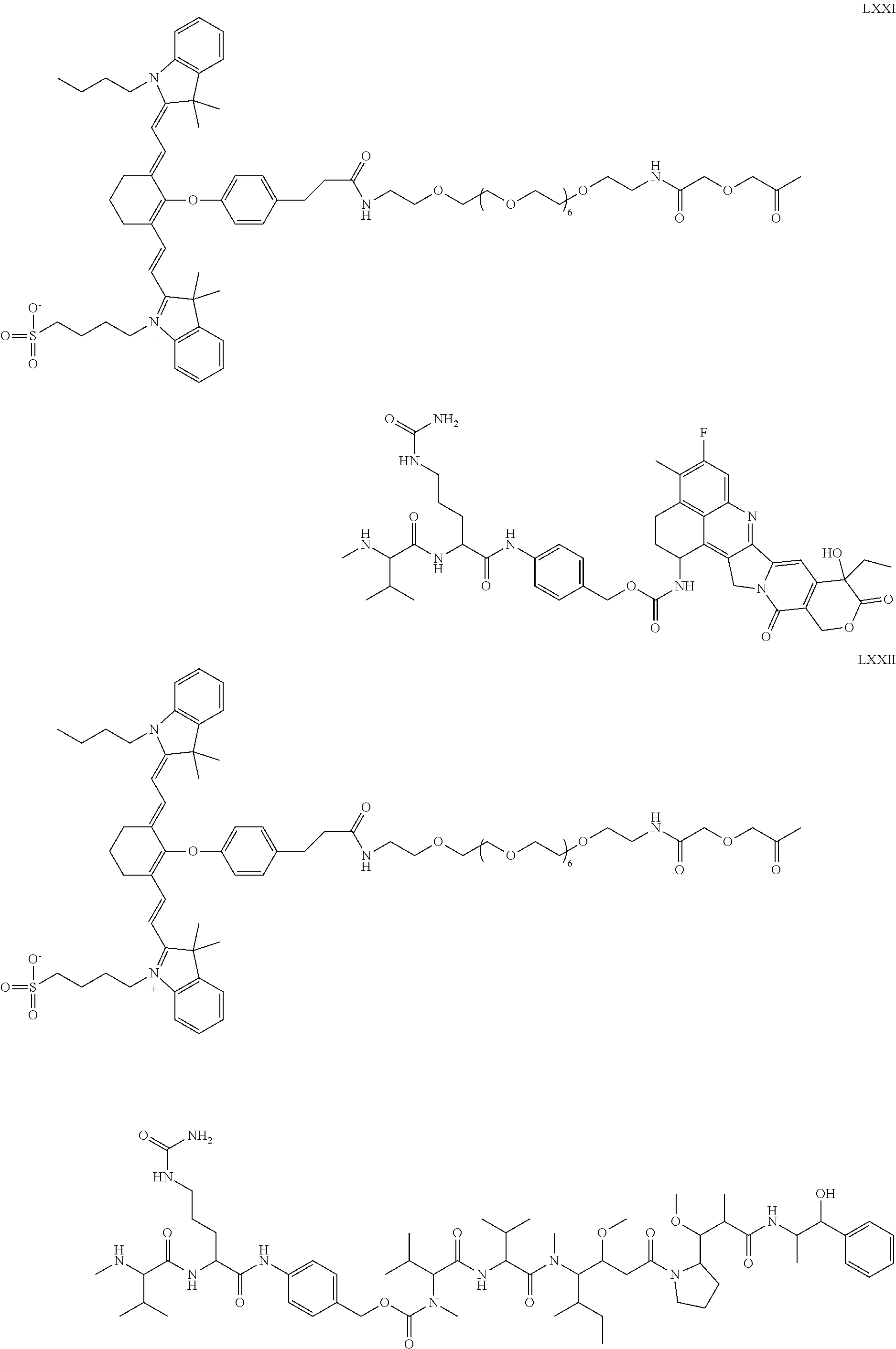

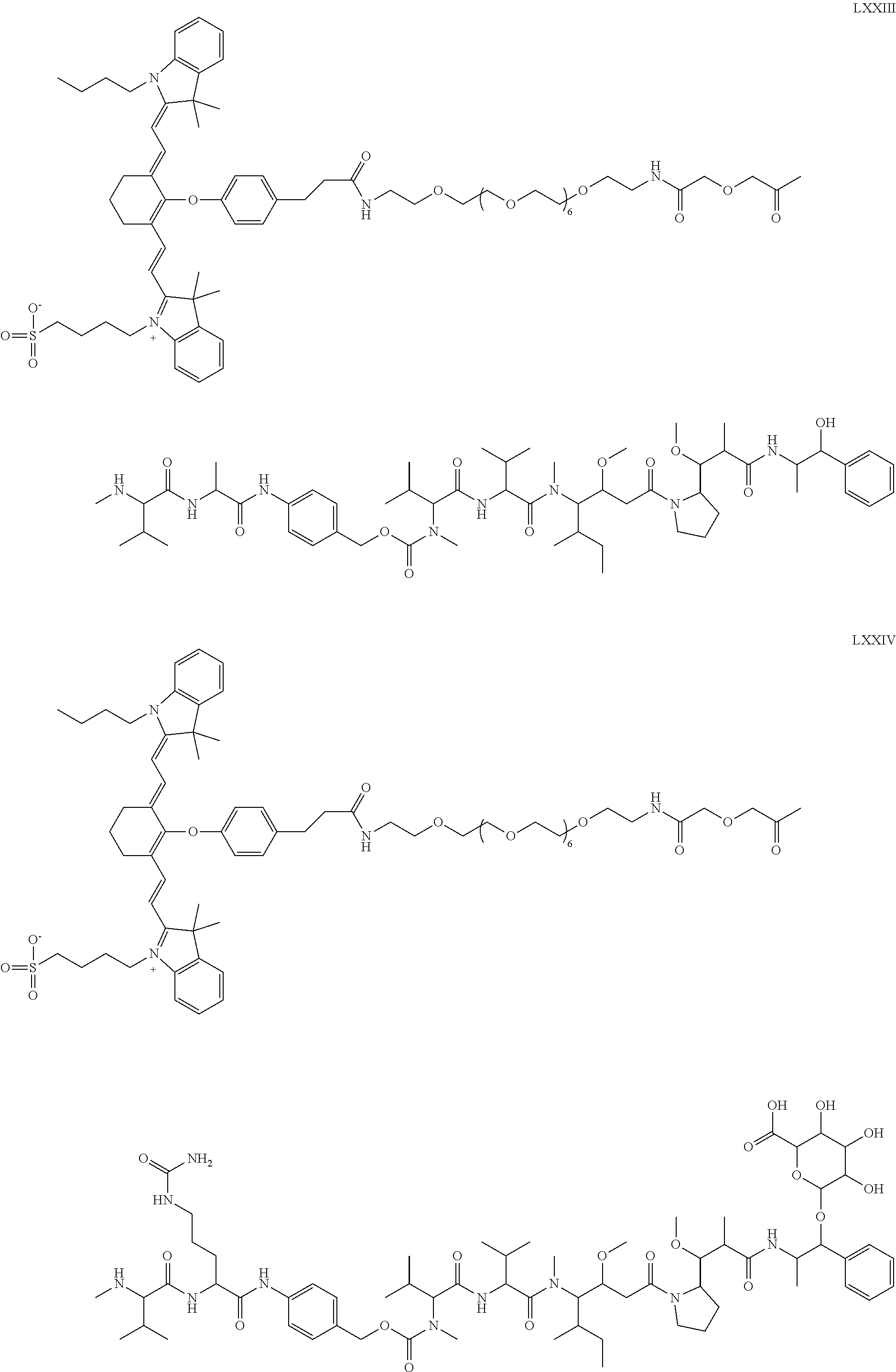

Exemplary conjugates comprising a tumor-targeting NIR dye and specifically, the dye shown in Formula IX, coupled to a chemotherapeutic agent via a linker including a cleavable peptide portion which may be useful with the present invention are shown in Formulas LXXV through LXXVII.

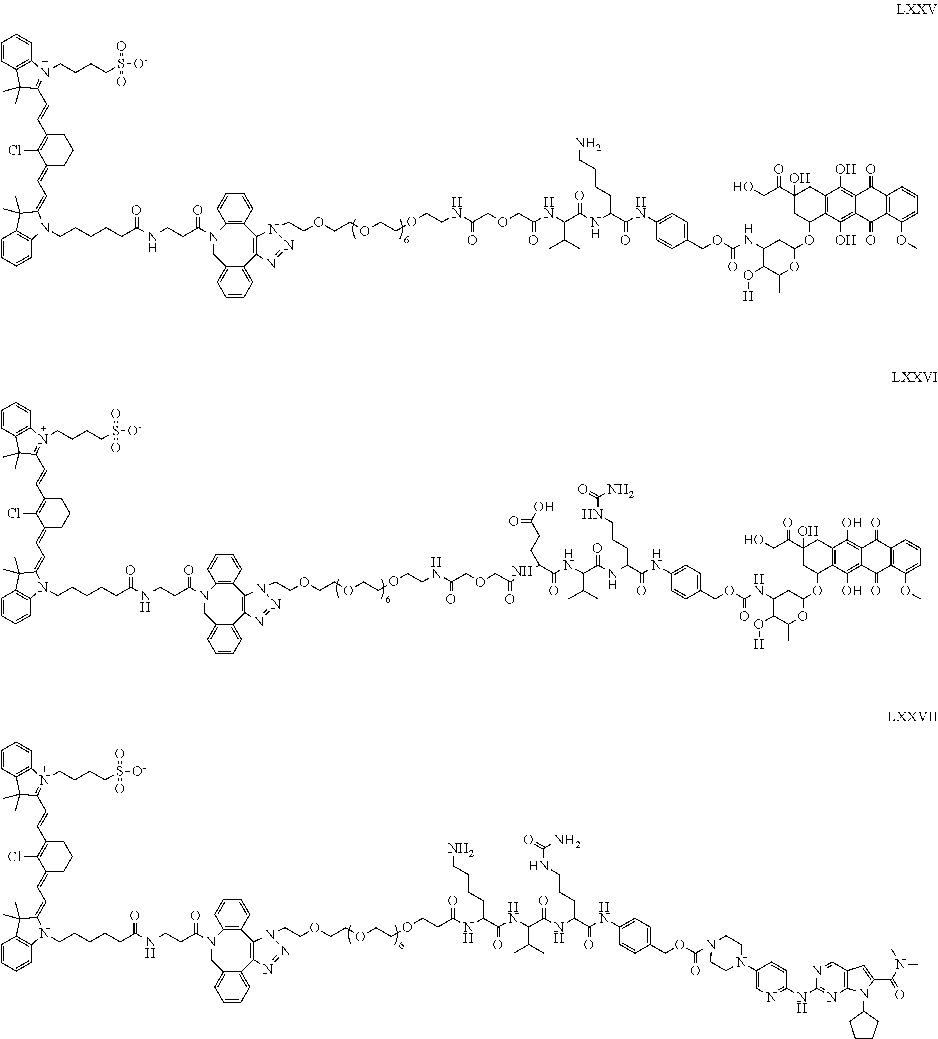

An exemplary conjugate comprising a tumor-targeting NIR dye and specifically, the dye shown in Formula XV, coupled to a chemotherapeutic agent via a linker including a cleavable peptide portion which may be useful with the present invention is shown in Formula LXXVIII.

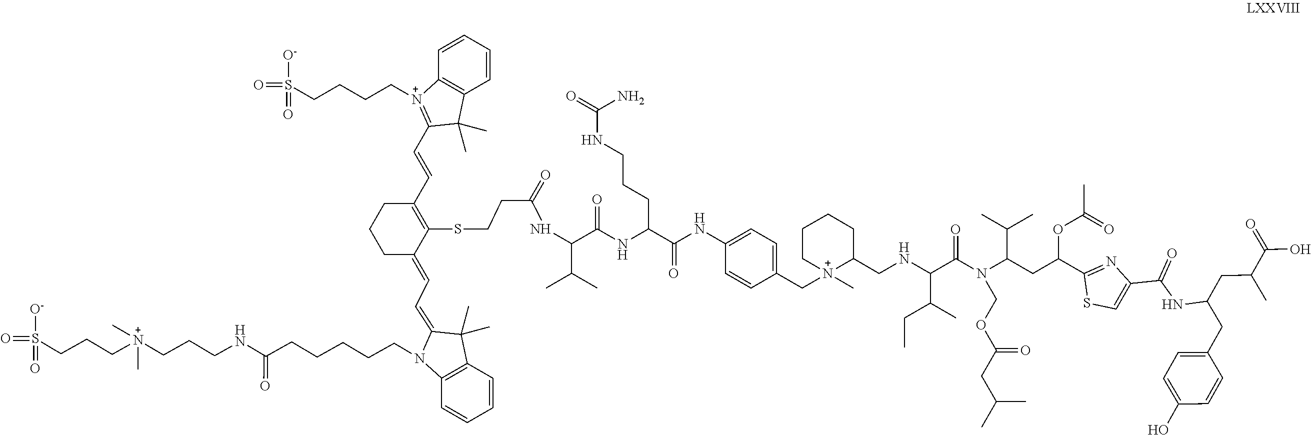 [SMILES: CCC(C)C(NCC1CCCC[N+]1(C)Cc1ccc(NC(=O)C(CCCNC(N)=O)NC(=O)C(NC(=O)CCSC2=C(/C=C/C3=[N+](CCCCS(=O)(=O)[O-])c4ccccc4C3(C)C)CCC/C2=C\C=C2\N(CCCCCC(=O)NCCC[N+](C)(C)CCCS(=O)(=O)[O-])c3ccccc3C2(C)C)C(C)C)cc1)C(=O)N(COC(=O)CC(C)C)C(CC(OC(C)=O)c1nc(C(=O)NC(Cc2ccc(O)cc2)CC(C)C(=O)O)cs1)C(C)C]

As to particular embodiments, the linker can comprise a self-immolative (or self-eliminating or self-degrading) portion which may be useful to conjugate a tumor-targeting NIR dye and a chemotherapeutic agent as per the present invention.

Now referring primarily to FIGS. 7B, 7C, 7D, and 7F, as to particular embodiments, a first self-immolative portion can be used with the present conjugate to link a dye and a chemotherapeutic agent. As to particular embodiments, a first self-immolative portion can be useful to link a cleavable peptide portion and a chemotherapeutic agent, whereby the first self-immolative portion may function to space the cleavable peptide portion and the chemotherapeutic agent to relieve steric hinderance and thus allow a protease to access the protease-sensitive cleavable peptide portion for cleavage thereof. Following cleavage of the cleavable peptide portion, a specific stimulus (such as an enzyme, pH change, etc.) can trigger the first self-immolative portion to release the chemotherapeutic agent and correspondingly deliver the conjugate's payload proximate a target tumor cell population.

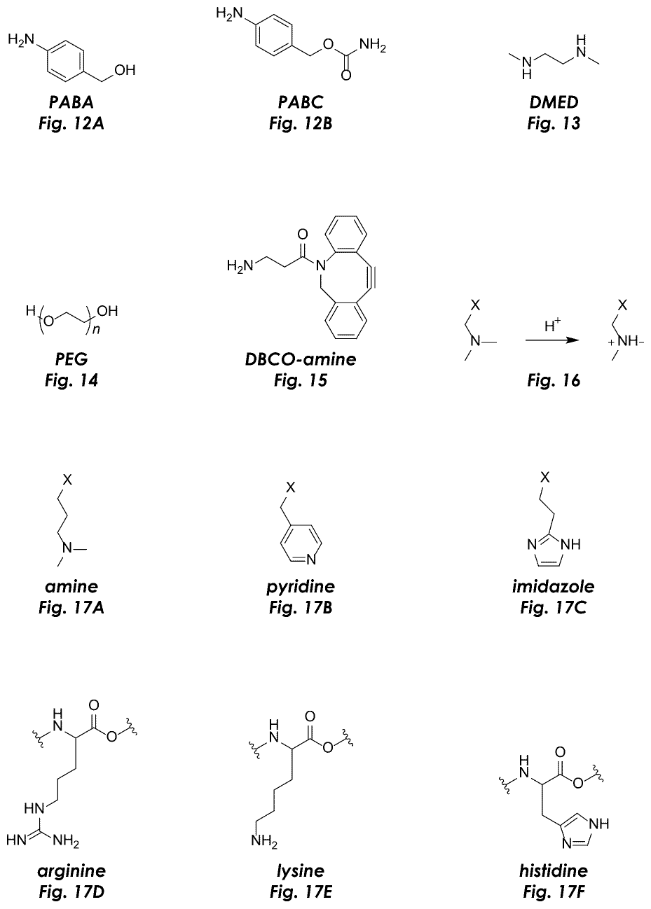

As to particular embodiments, illustrative examples of first self-immolative portions which may be useful with the present invention include para-aminobenzyl alcohol (PABA) and para-aminobenzyl carbamate (PABC), as shown in FIGS. 12A and 12B, respectively. Following cleavage of the cleavable peptide portion and exposure to an acidic environment, both PABA and PABC can undergo a 1,6-elimination reaction to release the chemotherapeutic agent.

Now referring primarily to FIGS. 7C and 7D, as to particular embodiments, a second self-immolative portion can be used with the present conjugate to link a dye and a chemotherapeutic agent. As to particular embodiments, a second self-immolative portion can be useful to link a first self-immolative portion and a chemotherapeutic agent. Following self-elimination of the first self-immolative portion, a specific stimulus (such as an enzyme, pH change, etc.) can trigger the second self-immolative portion to release the chemotherapeutic agent and correspondingly deliver the conjugate's payload proximate a target tumor cell population.

As to particular embodiments, an illustrative example of a second self-immolative portion which may be useful with the present invention includes N,N-dimethylethylenediamine (DMED), as shown in FIG. 13. Following self-elimination of the first self-immolative portion, DMED can undergo a cyclization reaction to form 1,3-dimethyl-2-imidazolidinone and release the chemotherapeutic agent. As to particular embodiments, DMED may be particularly useful between cleavable peptide portions comprising glucuronic acid and chemotherapeutic agents having a linkable hydroxyl group.

As to particular embodiments, a solubility-modifying portion can be used with the present conjugate to link a dye and a chemotherapeutic agent to achieve the desired solubility of the conjugate. Now referring primarily to FIGS. 7D through 7F, as to particular embodiments, due to the hydrophobic nature of the dye and/or the chemotherapeutic agent and/or some peptides, the solubility-modifying portion can be a hydrophilic portion which may link the dye and the cleavable linker, whereby the hydrophilic portion does not interfere with the drug release mechanism. As to particular embodiments, an illustrative example of a hydrophilic portion which may be useful with the present invention is shown in Formula LXXIX.

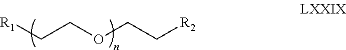

wherein:
R₁ can be any functional group which can form a stable bond with the dye;
R₂ can be any functional group which can form a stable bond with the cleavable portion of the linker; and
n can be 0 to 45.

As to particular embodiments, an illustrative example of a hydrophilic portion which may be useful with the present invention includes polyethylene glycol (PEG) (as shown in FIG. 14) with various chain lengths, whereby PEG may increase solubility in aqueous mediums and provide the desired drug metabolism and pharmacokinetics (DMPK) of the delivered chemotherapeutic agent. Notably, a longer PEG chain generally leads to greater hydrophilicity.

As to particular embodiments, click chemistry can be used to link the dye and the linker, such as the hydrophilic portion of the linker or the cleavable portion of the linker. As but one illustrative example, dibenzocyclooctyne (DBCO)-containing reagents, as such dibenzocyclooctyne-amine (as shown in FIG. 15), may be useful with the present invention for click chemistry. These reagents include a highly reactive DBCO group which readily reacts with azide-tagged molecules or biomolecules to form a stable triazole ring without the need for a copper catalyst.

As to particular embodiments, the present conjugate can include a dye linked to two chemotherapeutic agents, whether the same or different.

Exemplary conjugates comprising a tumor-targeting NIR dye coupled to two chemotherapeutic agents via respective linkers, each including a cleavable peptide portion, which may be useful with the present invention are shown in Formulas LXXX through LXXXIII.

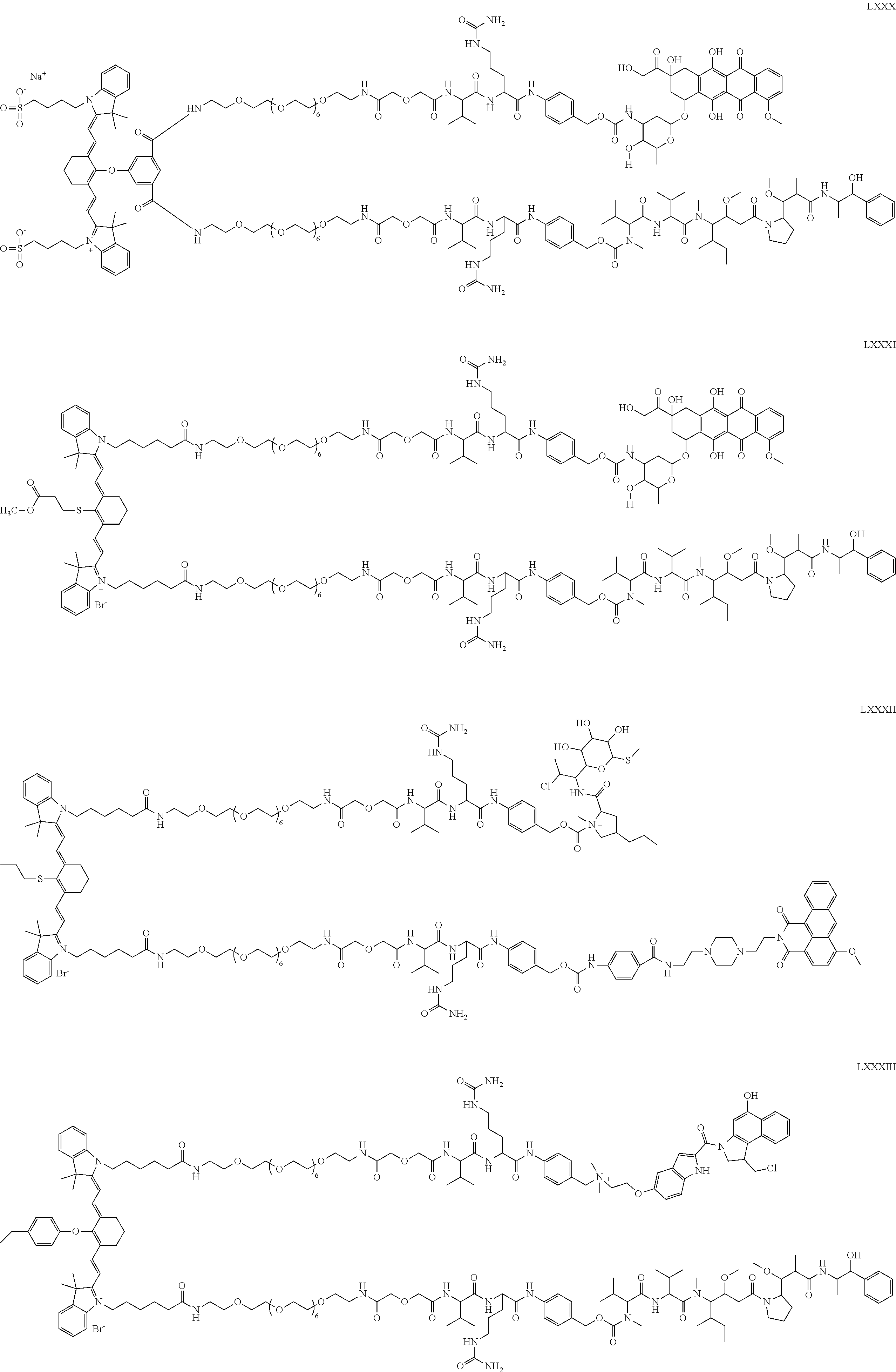

As to particular embodiments, the tumor-targeting NIR dye or the linker can be modified to have a basic functionality, whereby such a modification may include the attachment of one or more basic compounds to the dye or the linker or the incorporation of one or more basic compounds into the dye or the linker. Each basic compound can function as a base which may accept a proton (H⁺) (as shown in FIG. 16) and thus be protonated in an acidic environment, such as a tumor microenvironment which can be acidic as a result of increased glycolytic metabolism and limited oxygen and nutrient availability that may lead to the buildup of lactic acid and other acidic metabolites. Protonation of the basic compound proximate the tumor microenvironment can enhance penetration into the tumor via attraction of its positive charge to the negative charges on tumor cell membranes for uptake and retention. Additionally, in the circulation, the basic functionality can impart a relatively neutral charge to the dye or the linker which may preclude interaction with positively or negatively charged proteins in the blood and subsequent recognition and clearance by the macrophage scavenger system.

As a first example, basic functional groups which may be useful with the present invention include amines, sulfonamides, and imidazoles.

As a second example, basic functional groups which may be useful with the present invention can include those shown in FIGS. 17A through 17F.

Exemplary dyes having a basic functional group which may be useful with the present invention are shown in Formulas LXXXIV through XCII.

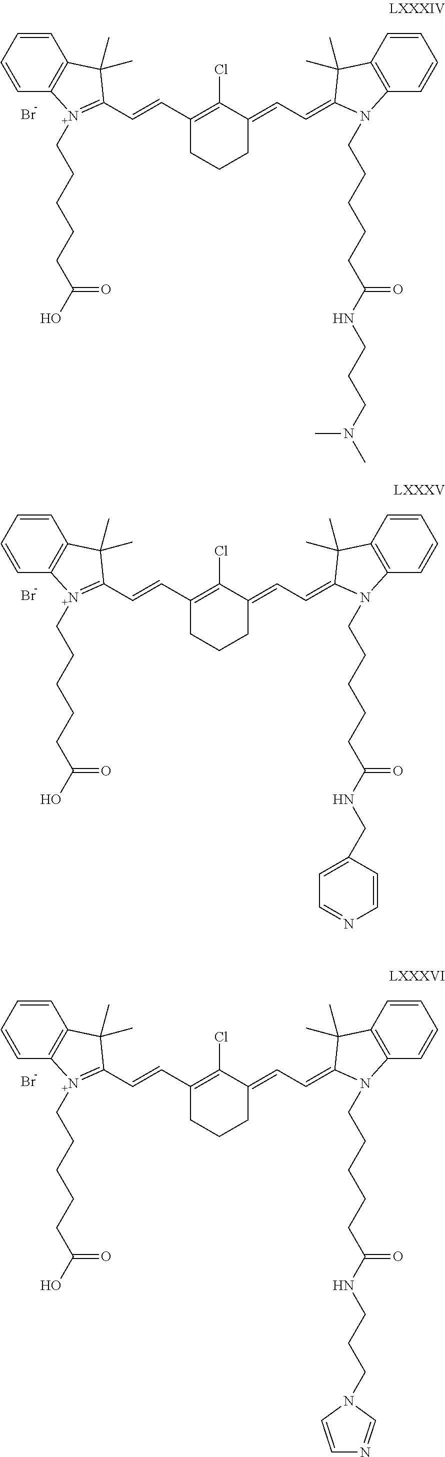 [SMILES: CN(C)CCCNC(=O)CCCCCN1/C(=C/C=C2\CCCC(/C=C/C3=[N+](CCCCCC(=O)O)c4ccccc4C3(C)C)=C2Cl)C(C)(C)c2ccccc21 ; Br ; CC1(C)C(/C=C/C2=C(Cl)C(=C/C=C3/N(CCCCCC(=O)NCc4ccncc4)c4ccccc4C3(C)C)/CCC2)=[N+](CCCCCC(=O)O)c2ccccc21 ; Br ; CC1(C)C(/C=C/C2=C(Cl)C(=C/C=C3/N(CCCCCC(=O)NCCCn4ccnc4)c4ccccc4C3(C)C)/CCC2)=[N+](CCCCCC(=O)O)c2ccccc21 ; Br]

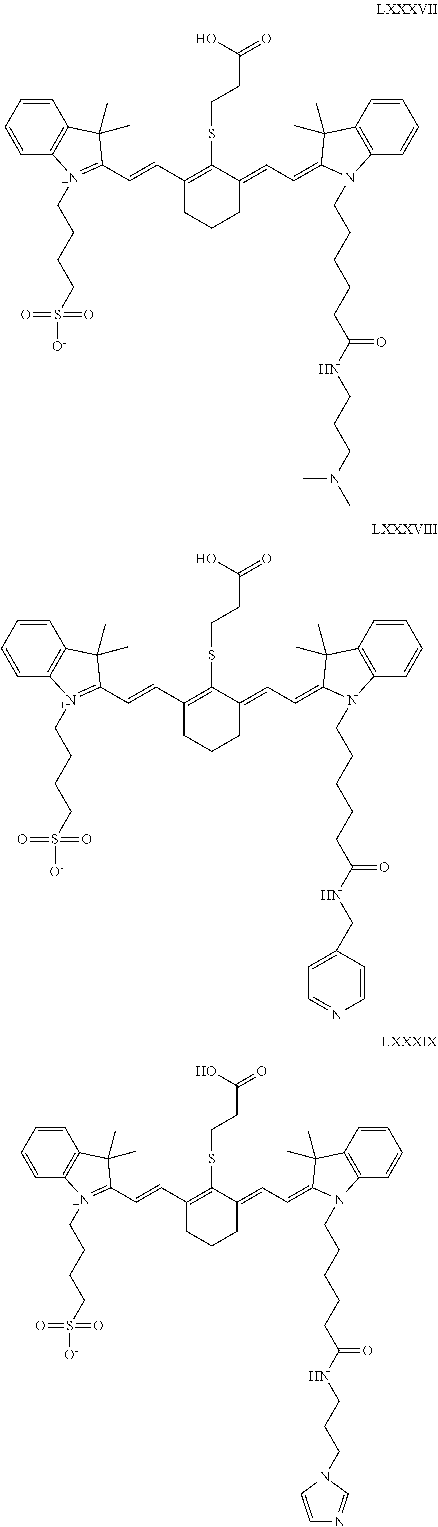 [SMILES: CN(C)CCCNC(=O)CCCCCN1/C(=C/C=C2\CCCC(/C=C/C3=[N+](CCCCS(=O)(=O)[O-])c4ccccc4C3(C)C)=C2SCCC(=O)O)C(C)(C)c2ccccc21 ; CC1(C)C(/C=C/C2=C(SCCC(=O)O)C(=C/C=C3/N(CCCCCC(=O)NCc4ccncc4)c4ccccc4C3(C)C)/CCC2)=[N+](CCCCS(=O)(=O)[O-])c2ccccc21 ; CC1(C)C(/C=C/C2=C(SCCC(=O)O)C(=C/C=C3/N(CCCCCC(=O)NCCCn4ccnc4)c4ccccc4C3(C)C)/CCC2)=[N+](CCCCS(=O)(=O)[O-])c2ccccc21]

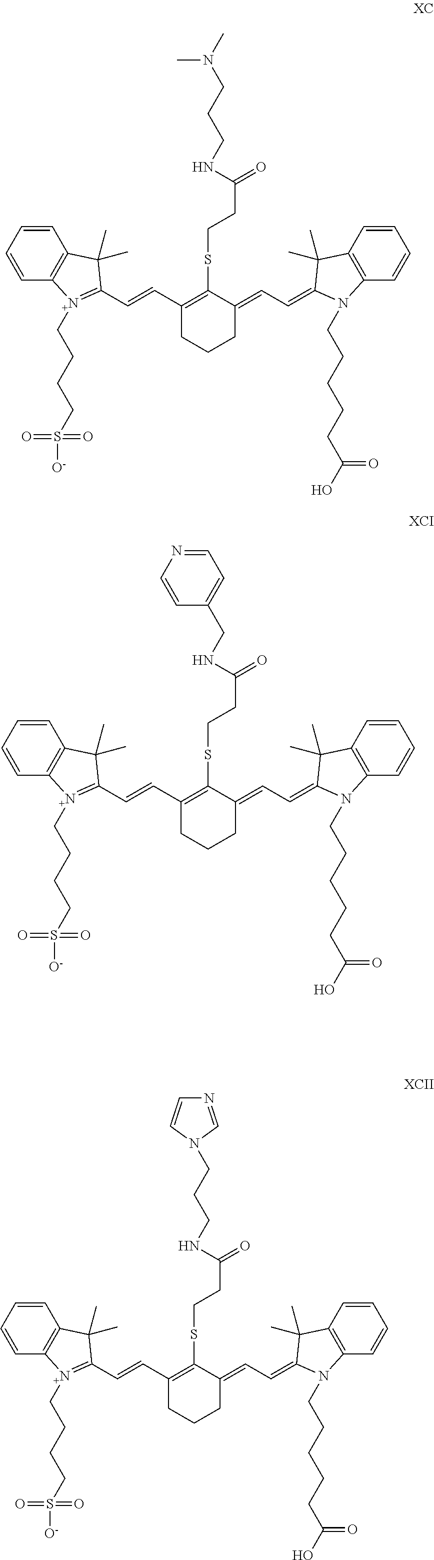 [SMILES: CN(C)CCCNC(=O)CCSC1=C(/C=C/C2=[N+](CCCCS(=O)(=O)[O-])c3ccccc3C2(C)C)CCC/C1=C\C=C1\N(CCCCCC(=O)O)c2ccccc2C1(C)C ; CC1(C)C(/C=C/C2=C(SCCC(=O)NCc3ccncc3)C(=C/C=C3/N(CCCCCC(=O)O)c4ccccc4C3(C)C)/CCC2)=[N+](CCCCS(=O)(=O)[O-])c2ccccc21 ; CC1(C)C(/C=C/C2=C(SCCC(=O)NCCCn3ccnc3)C(=C/C=C3/N(CCCCCC(=O)O)c4ccccc4C3(C)C)/CCC2)=[N+](CCCCS(=O)(=O)[O-])c2ccccc21]

An exemplary conjugate comprising a tumor-targeting NIR dye having a basic functional group and specifically, the dye shown in Formula LXXXIV, coupled to a chemotherapeutic agent via a linker including a cleavable peptide portion which may be useful with the present invention is shown in Formula XCIII.

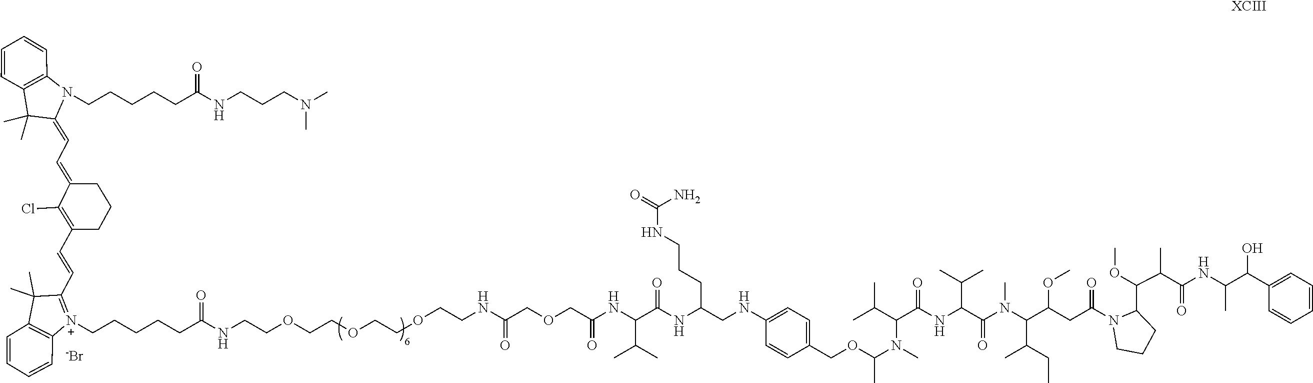

Exemplary conjugates comprising a tumor-targeting NIR dye having a basic functional group and specifically, the dye shown in Formula LXXXVII, coupled to a chemotherapeutic agent via a linker including a cleavable peptide portion which may be useful with the present invention are shown in Formulas XCIV and XCV.

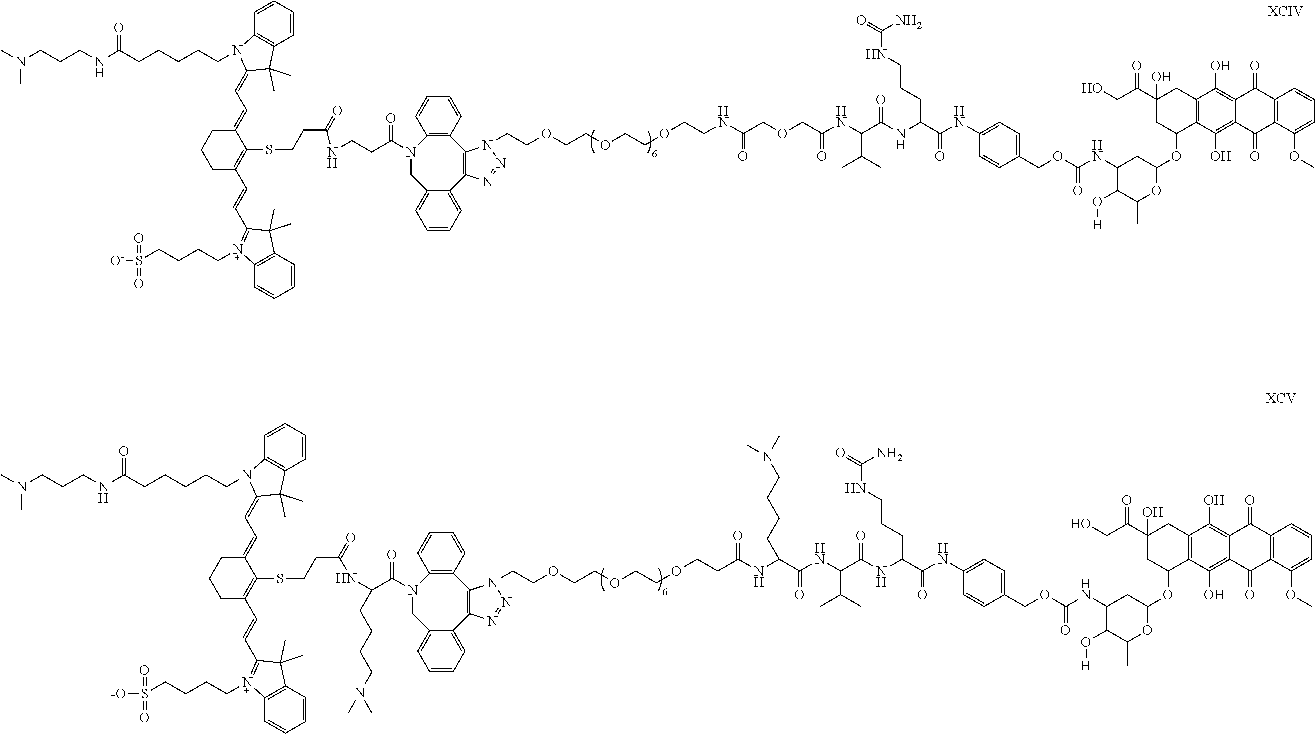

An exemplary conjugate comprising a tumor-targeting NIR dye having a basic functional group and specifically, the dye shown in Formula XC, coupled to a chemotherapeutic agent via a linker including a cleavable peptide portion which may be useful with the present invention is shown in Formula XCVI.

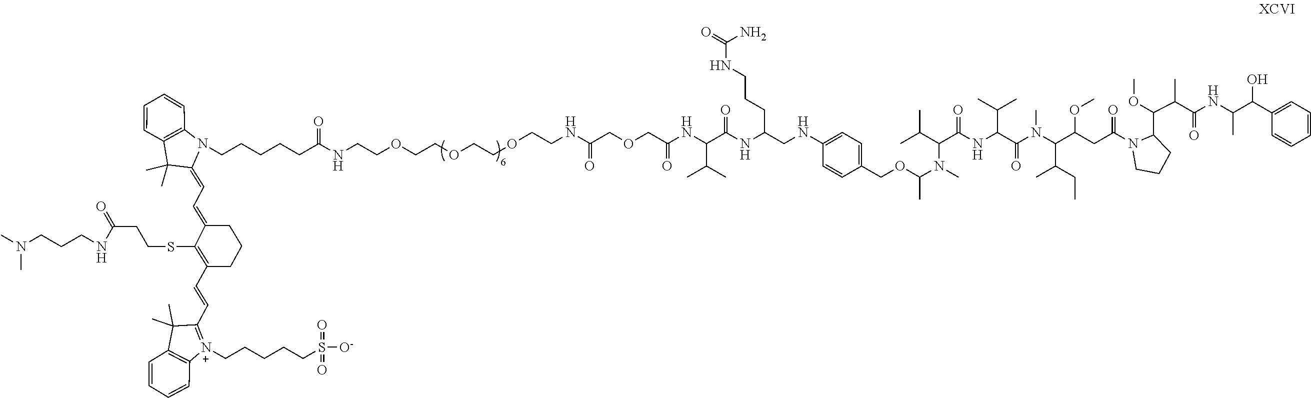

An exemplary conjugate comprising a tumor-targeting NIR dye having a basic functional group and specifically, the dye shown in Formula XCI, coupled to a chemotherapeutic agent via a linker including a cleavable peptide portion which may be useful with the present invention is shown in Formula XCVII.

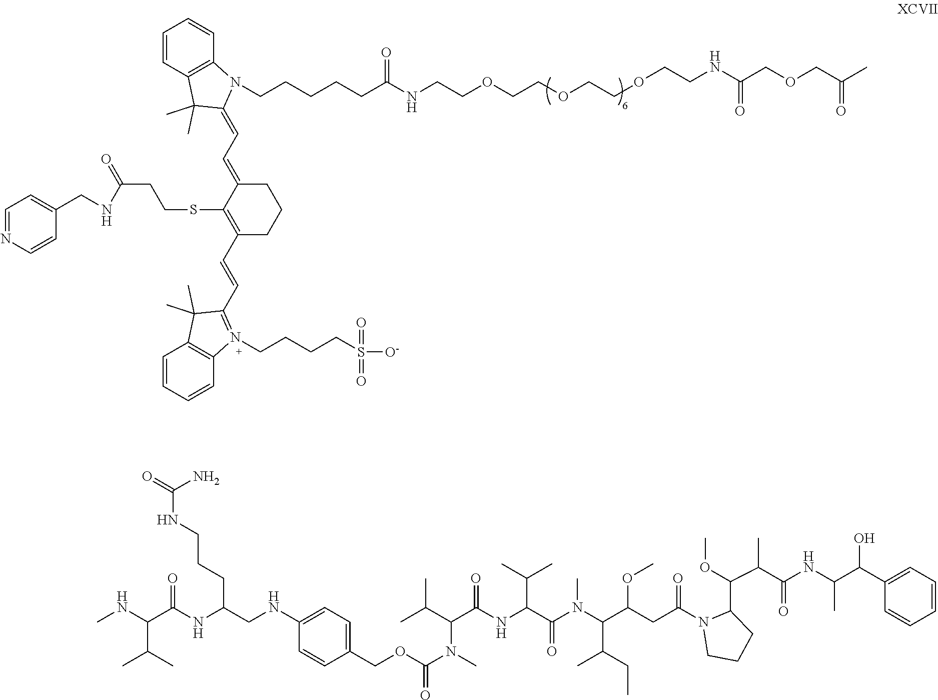

As to particular embodiments, as stated above, the linker can also be modified to have a basic functionality, whereby such a modification may include the attachment of one or more basic compounds to the linker or the incorporation of one or more basic compounds into the linker.

Exemplary linkers having a basic functional group and a cleavable peptide portion which may be useful with the present invention are shown in Formulas XCVIII through CVII.

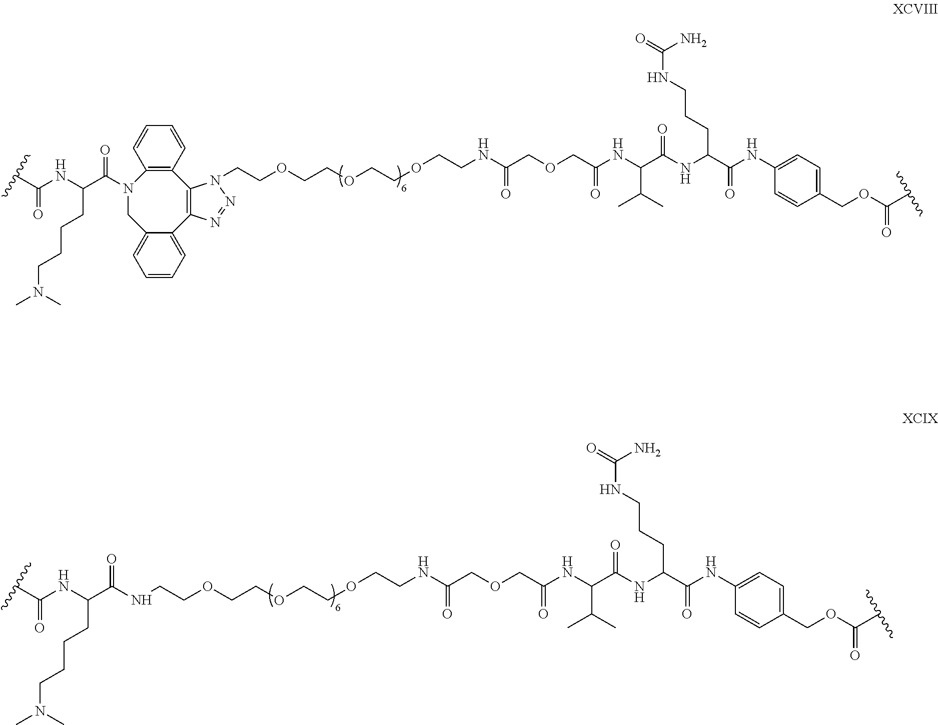

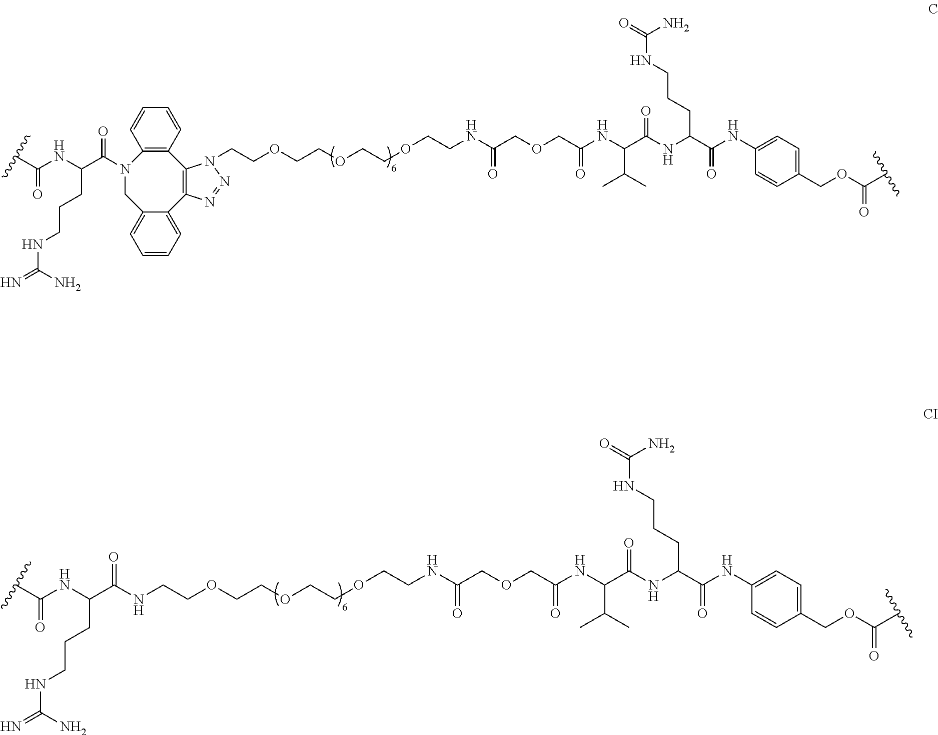

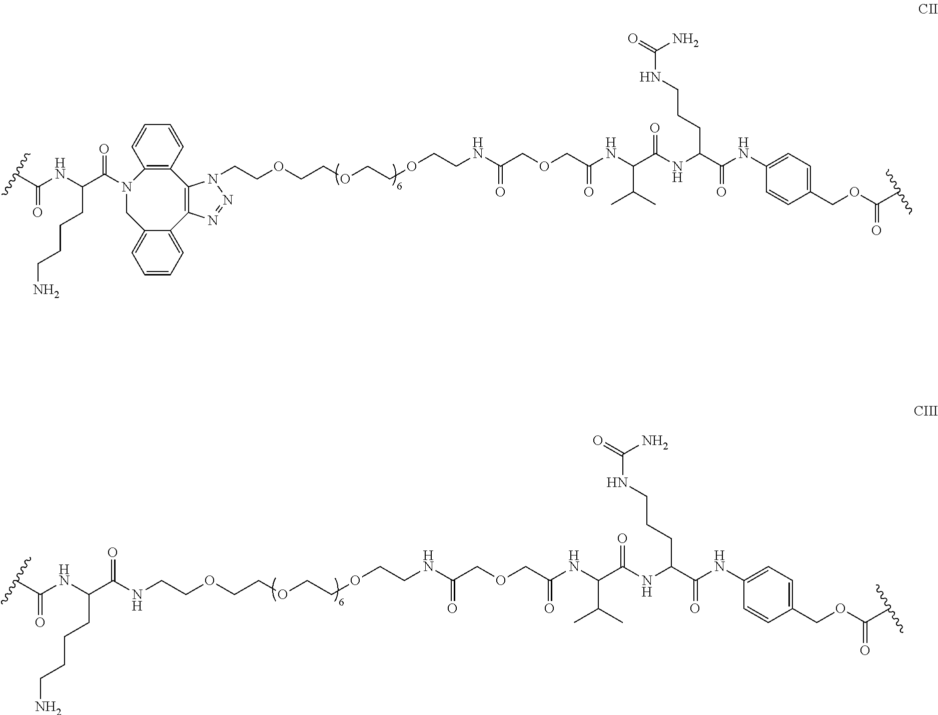

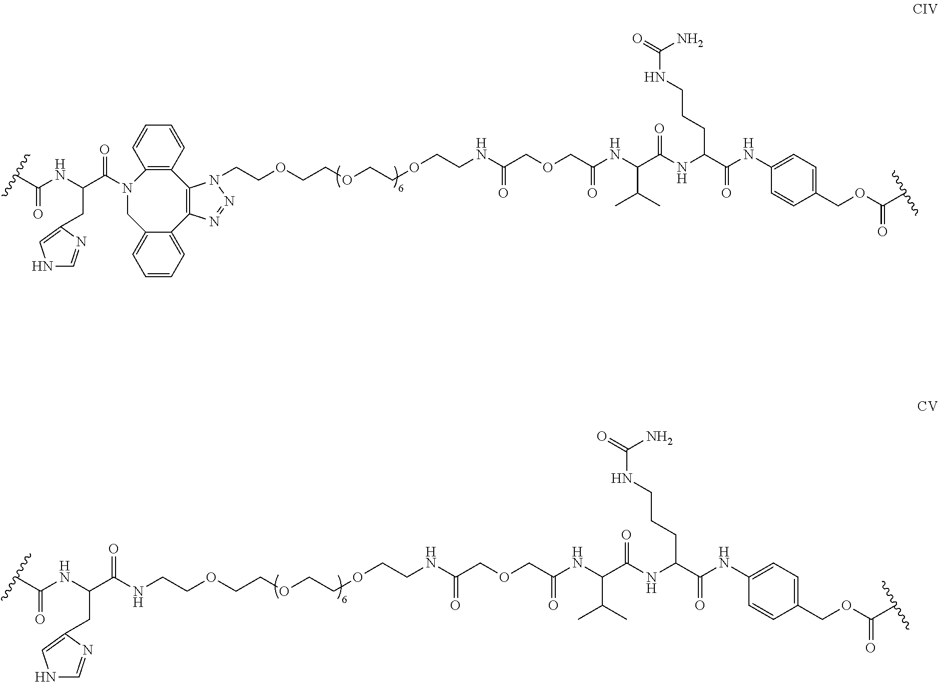

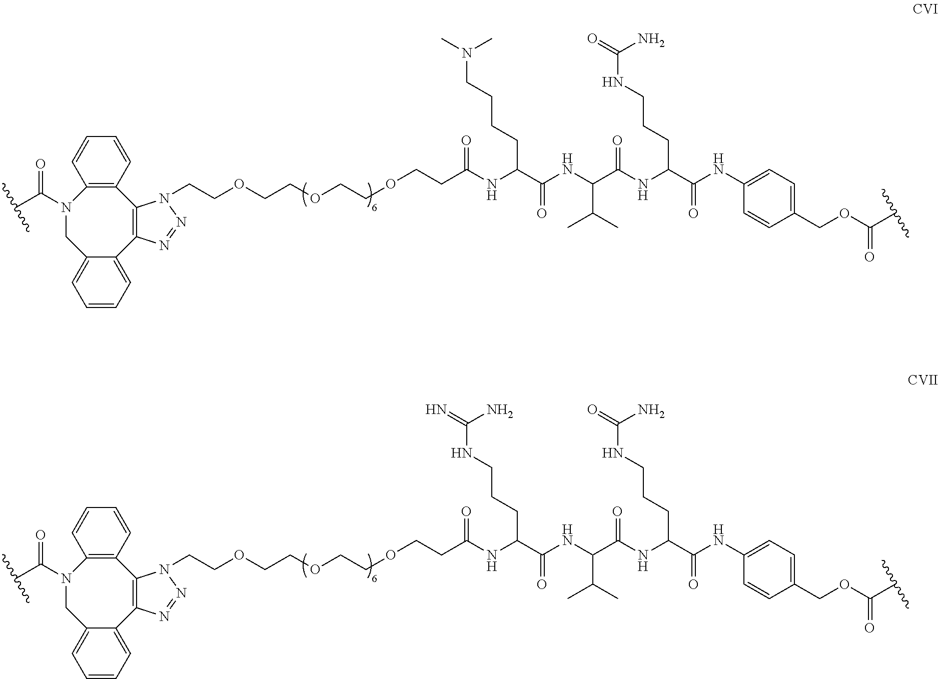

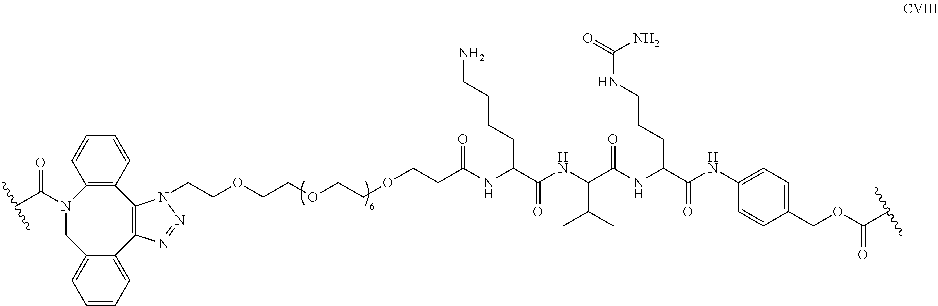

Exemplary conjugates comprising a tumor-targeting NIR dye, a linker having (i) a basic functional group and specifically, the linker shown in Formula XCVIII, and (ii) a cleavable peptide portion coupled to a chemotherapeutic agent which may be useful with the present invention are shown in Formulas CIX and CX.

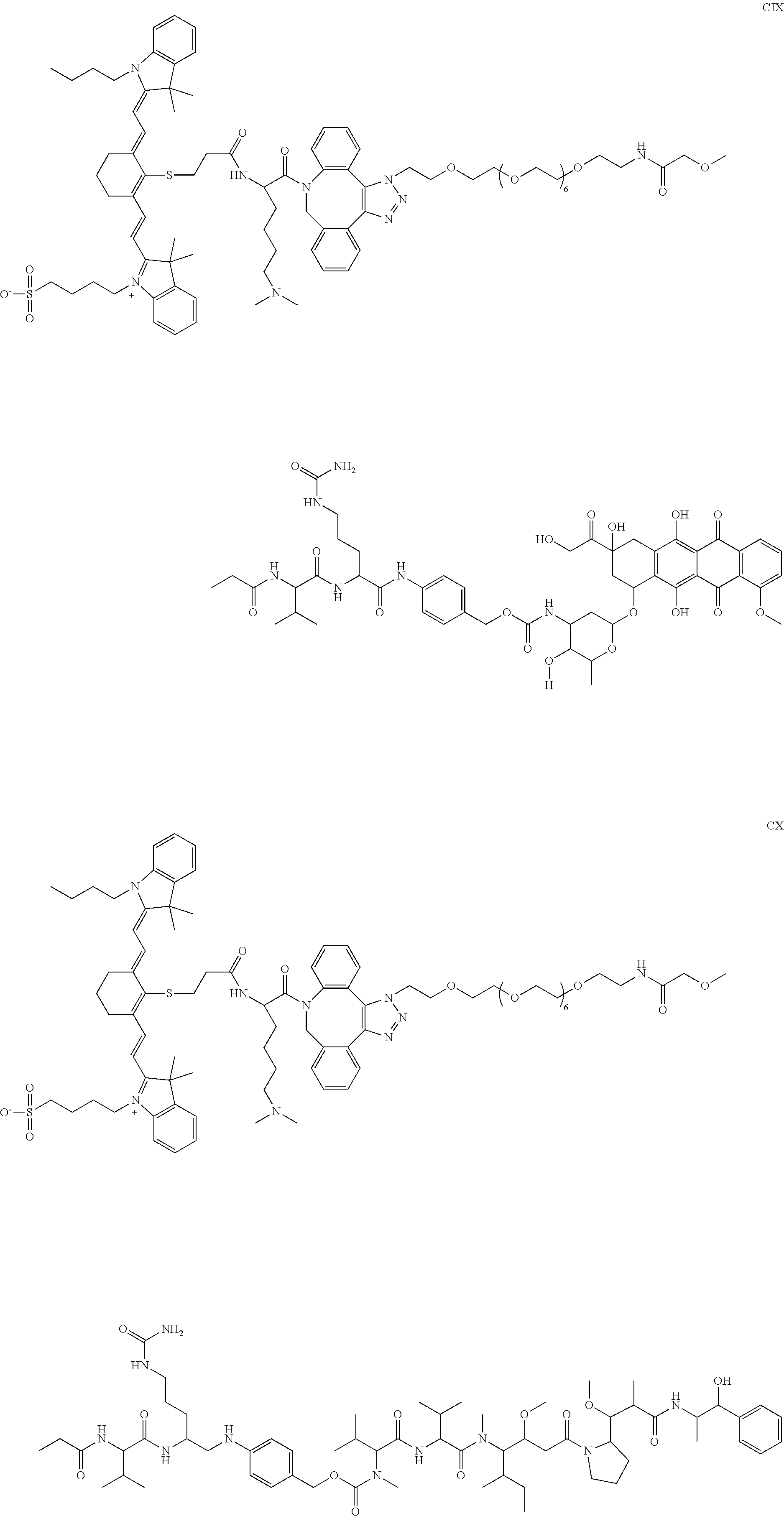

An exemplary conjugate comprising a tumor-targeting NIR dye, a linker having (i) a basic functional group and specifically, the linker shown in Formula C, and (ii) a cleavable peptide portion coupled to a chemotherapeutic agent which may be useful with the present invention is shown in Formula CXI.

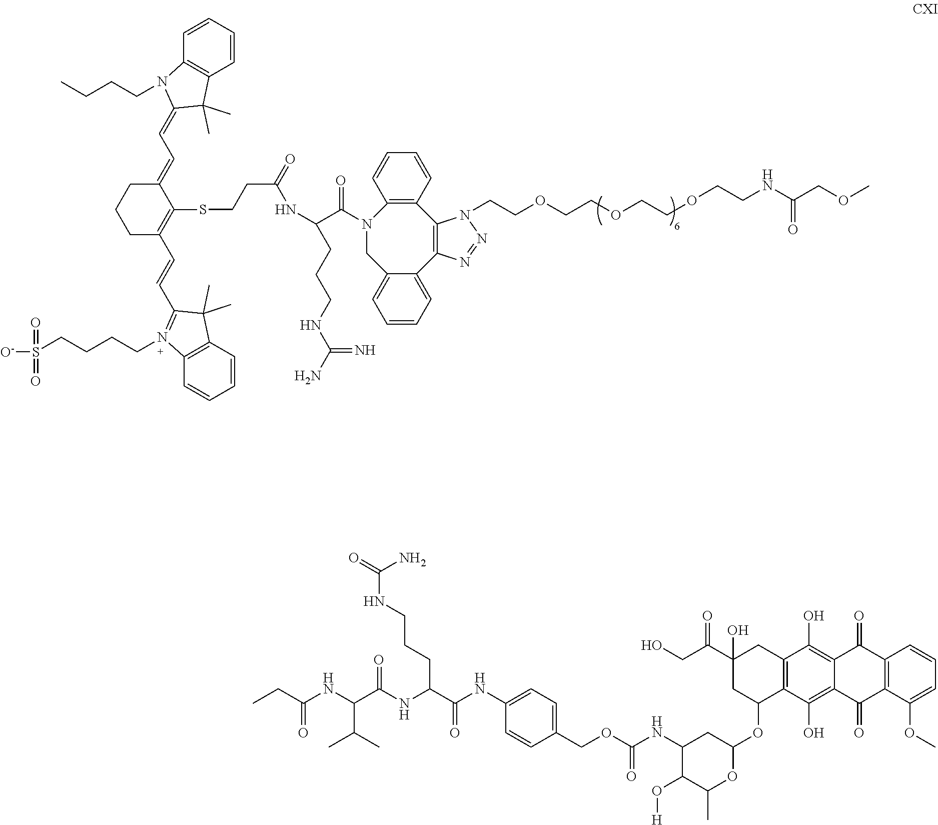

An exemplary conjugate comprising a tumor-targeting NIR dye, a linker having (i) a basic functional group and specifically, the linker shown in Formula CII, and (ii) a cleavable peptide portion coupled to a chemotherapeutic agent which may be useful with the present invention is shown in Formula CXII.

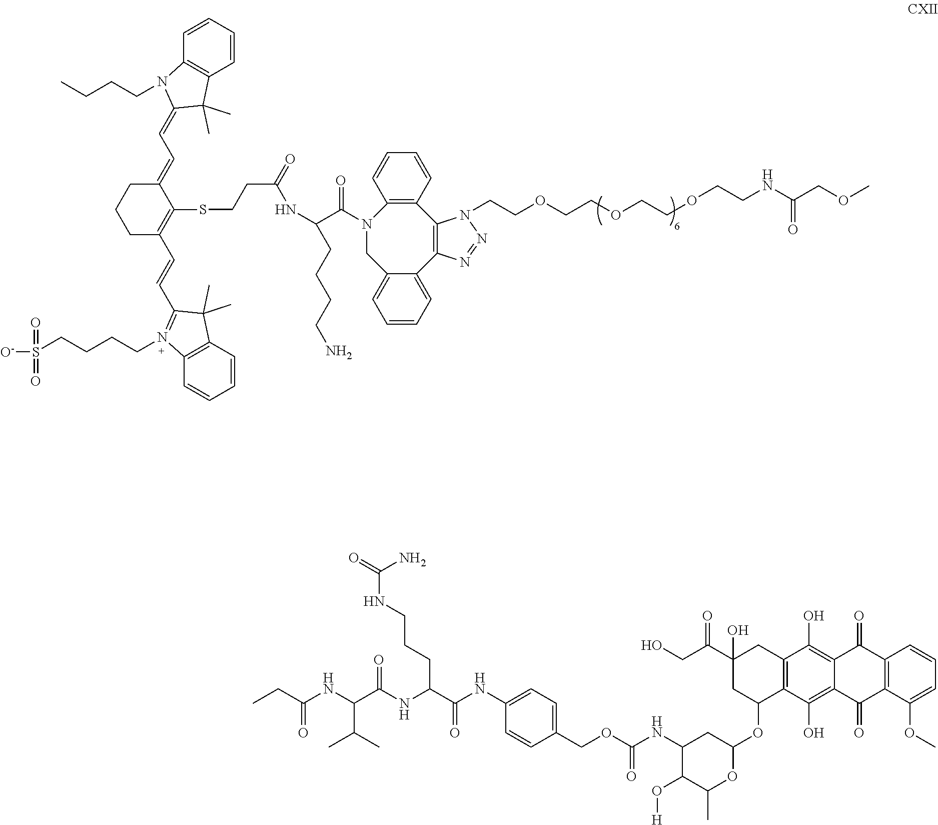

Exemplary conjugates comprising a tumor-targeting NIR dye, a linker having (i) a basic functional group and specifically, the linker shown in Formula CVI, and (ii) a cleavable peptide portion coupled to a chemotherapeutic agent which may be useful with the present invention are shown in Formulas CXIII through CXV.

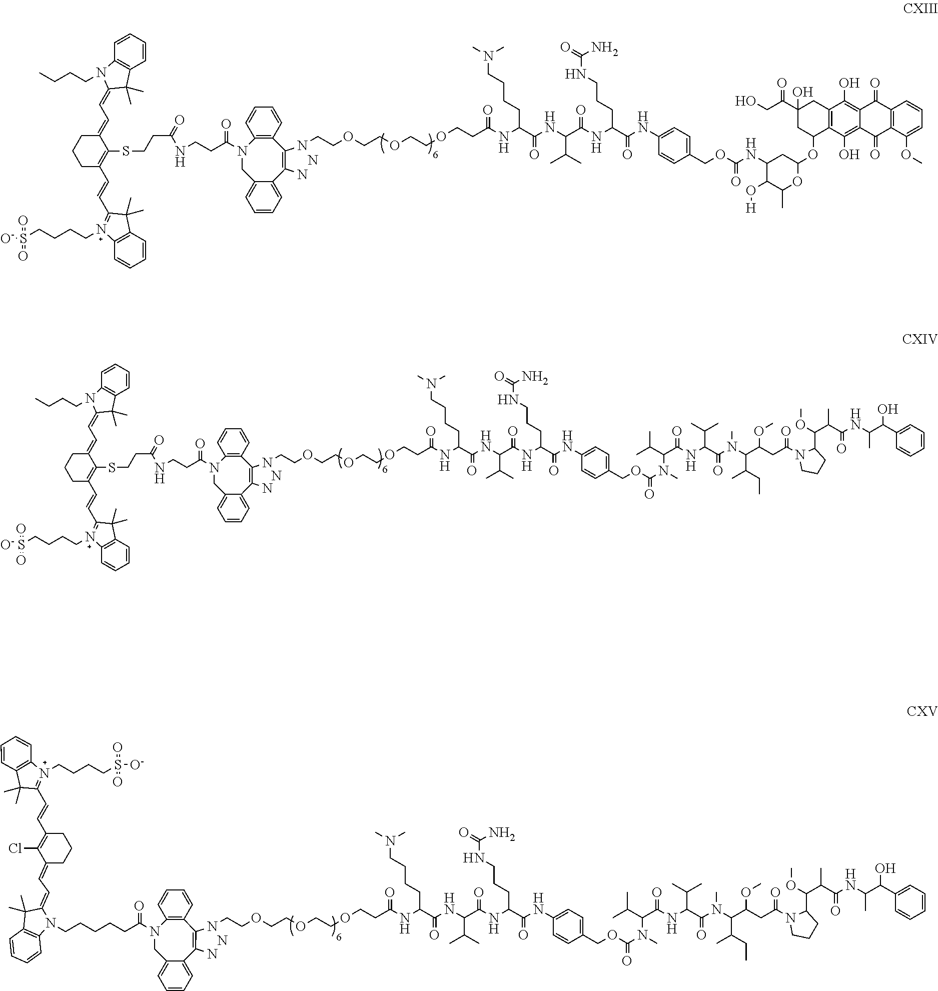

An exemplary conjugate comprising a tumor-targeting NIR dye, a linker having (i) a basic functional group and specifically, the linker shown in Formula CVII, and (ii) a cleavable peptide portion coupled to a chemotherapeutic agent which may be useful with the present invention is shown in Formula CXVI.

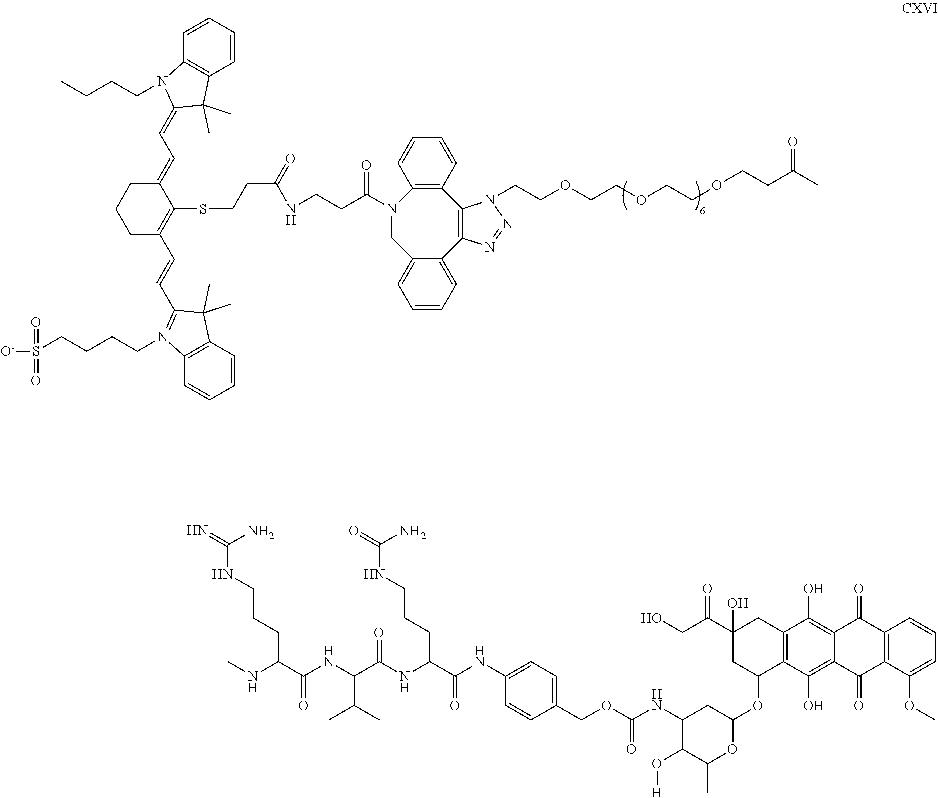

An exemplary conjugate comprising a tumor-targeting NIR dye, a linker having (i) a basic functional group and specifically, the linker shown in Formula CVIII, and (ii) a cleavable peptide portion coupled to a chemotherapeutic agent which may be useful with the present invention is shown in Formula CXVII.

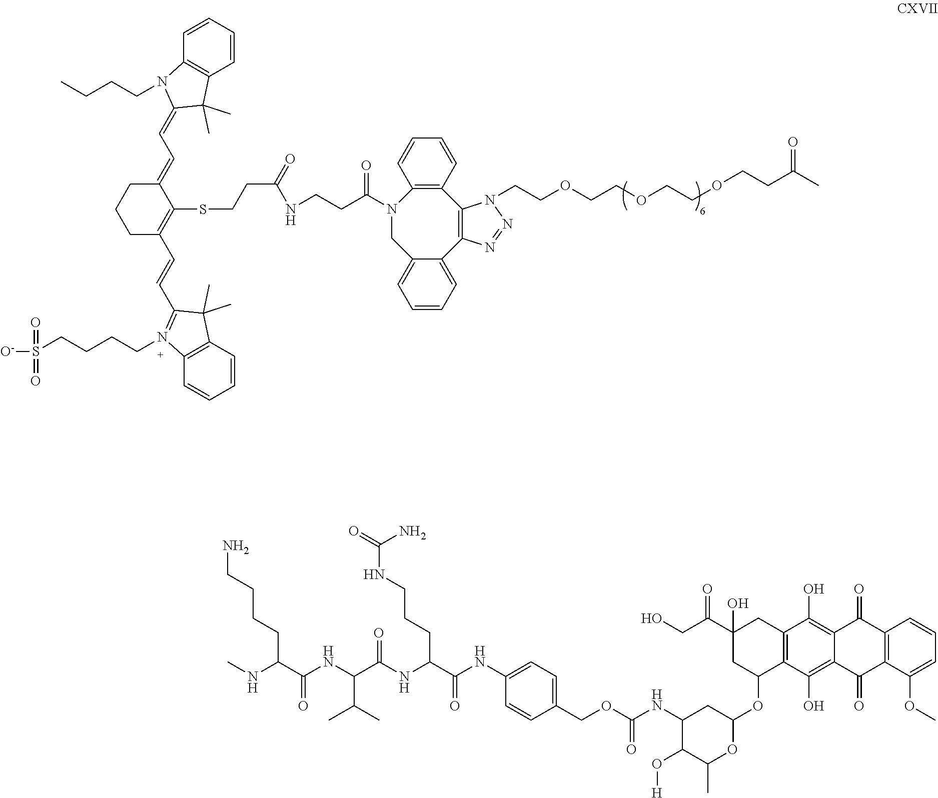

An exemplary conjugate comprising a tumor-targeting NIR dye, a linker having (i) two basic functional groups and (ii) a cleavable peptide portion coupled to a chemotherapeutic agent which may be useful with the present invention is shown in Formula CXVIII.

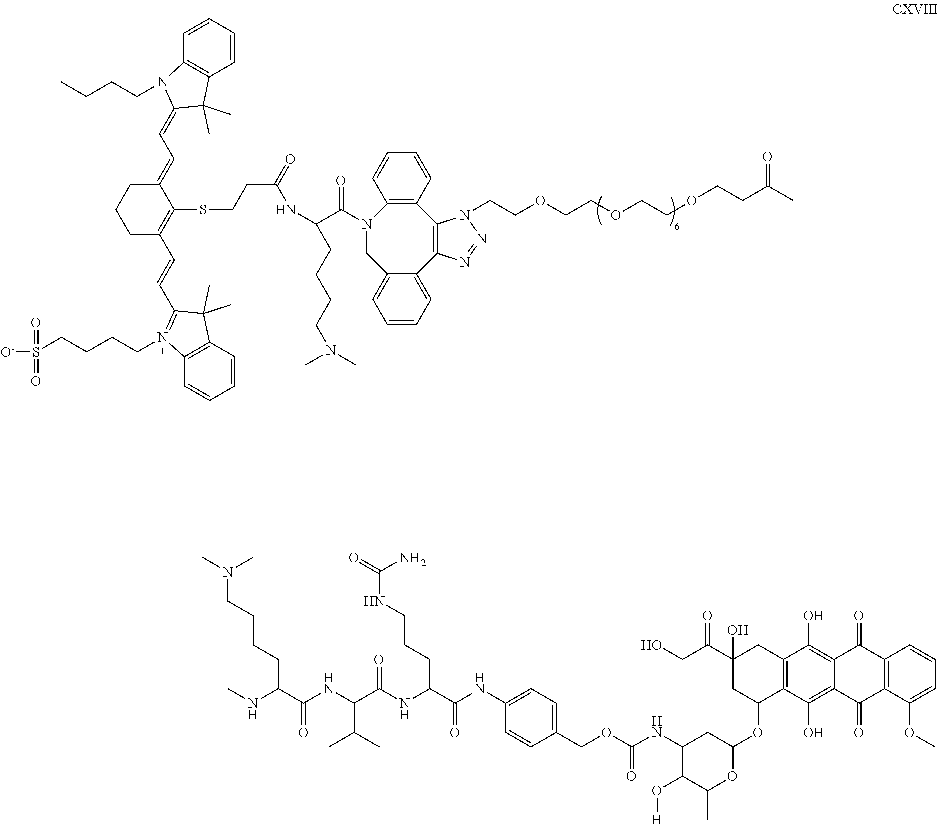

An exemplary conjugate comprising (i) a tumor-targeting NIR dye having a basic functional group and specifically, the dye shown in Formula LXXXVII, and (ii) a linker having two basic functional groups and a cleavable peptide portion coupled to a chemotherapeutic agent which may be useful with the present invention is shown in Formulas CXIX.

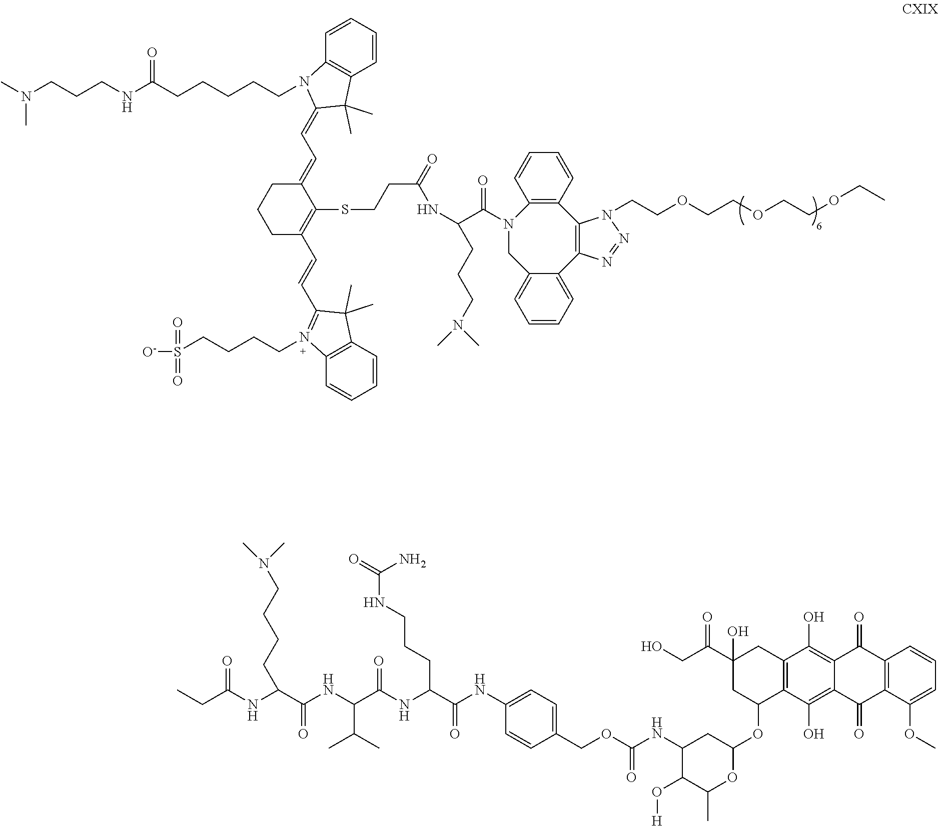

As to particular embodiments, the tumor-targeting NIR dye or the linker can be modified to have a functional group which targets tumor cell mitochondria, whereby such a modification may include the attachment of one or more lipophilic moieties and/or cationic moieties to the dye or the linker or the incorporation of one or more lipophilic moieties and/or cationic moieties into the dye or the linker, whereby the lipophilic moieties can readily cross the cell and mitochondrial membranes and the cationic moieties (such as with a delocalized positive charge) may be attracted by the negative charge within the mitochondrial matrix. As to particular embodiments, the mitochondria-targeting functional group can be both lipophilic and cationic.

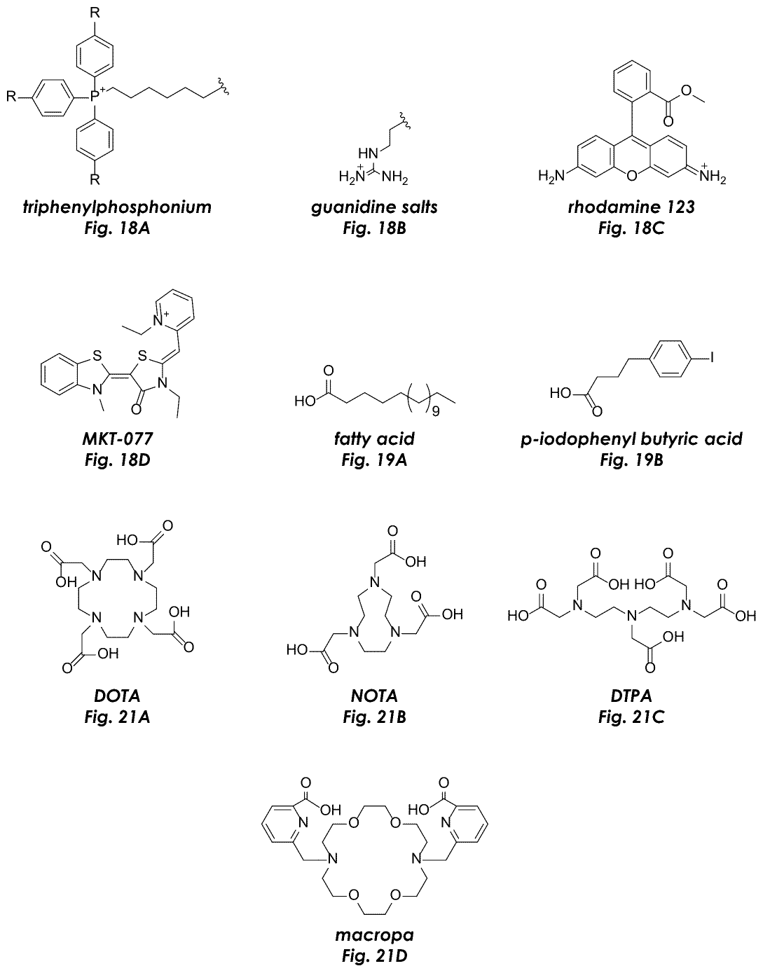

As illustrative examples, mitochondria-targeting functional groups which may be useful with the present invention can include those shown in FIGS. 18A through 18D.

Exemplary dyes having a mitochondria-targeting functional group which may be useful with the present invention are shown in Formulas CXX through CXXV.

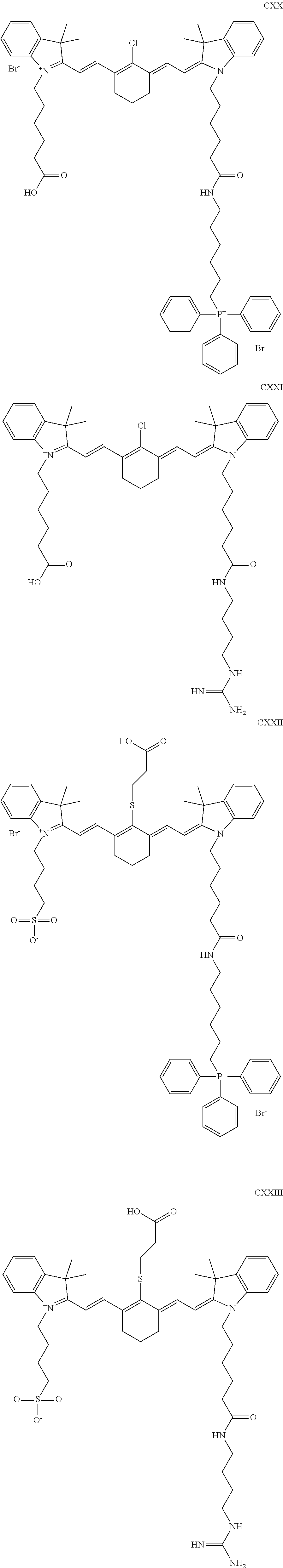

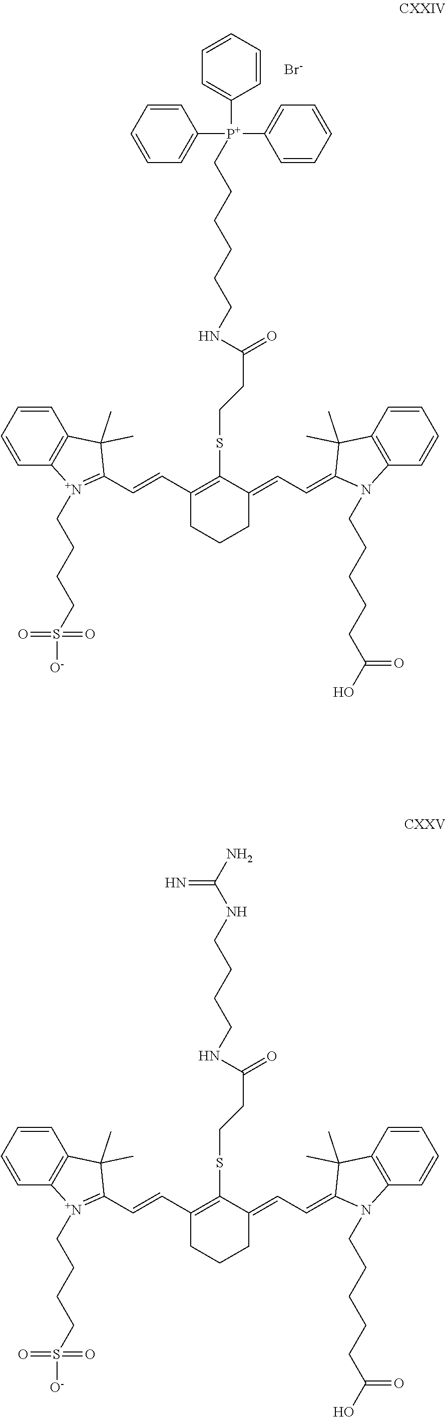 [SMILES: CC1(C)C(/C=C/C2=C(SCCC(=O)NCCCCCC[P+](c3ccccc3)(c3ccccc3)c3ccccc3)C(=C/C=C3/N(CCCCCC(=O)O)c4ccccc4C3(C)C)/CCC2)=[N+](CCCCS(=O)(=O)[O-])c2ccccc21 ; [Br-] ; CC1(C)C(/C=C/C2=C(SCCC(=O)NCCCCNC(=N)N)C(=C/C=C3/N(CCCCCC(=O)O)c4ccccc4C3(C)C)/CCC2)=[N+](CCCCS(=O)(=O)[O-])c2ccccc21]

An exemplary conjugate comprising a tumor-targeting NIR dye having a mitochondria-targeting functional group and specifically, the dye shown in Formula CXX, coupled to a chemotherapeutic agent via a linker including a cleavable peptide portion which may be useful with the present invention is shown in Formula CXXVI.

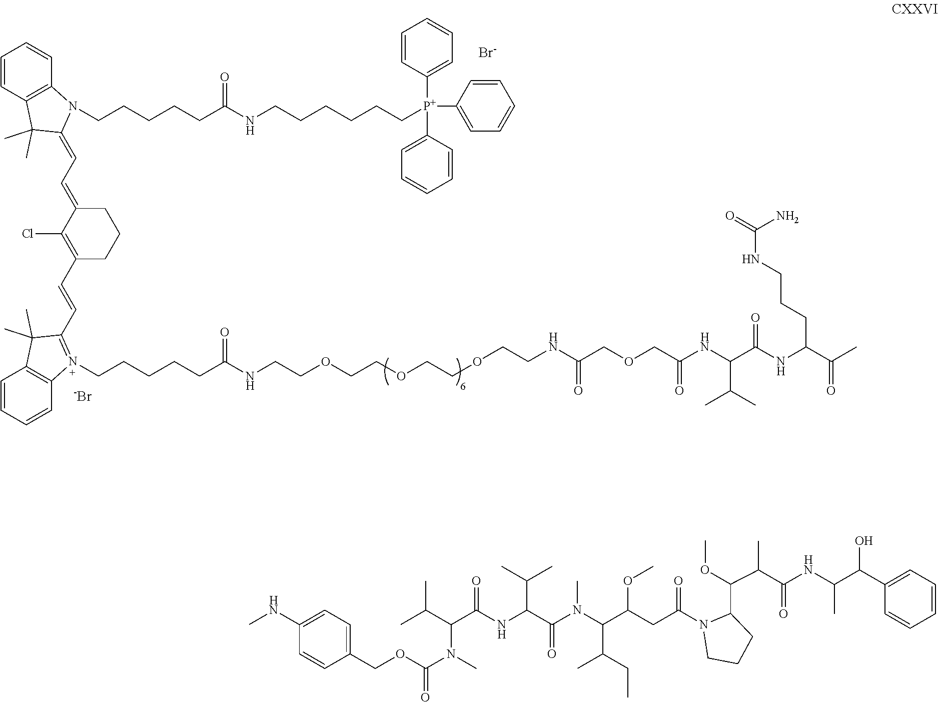

An exemplary conjugate comprising a tumor-targeting NIR dye having a mitochondria-targeting functional group and specifically, the dye shown in Formula CXXII, coupled to a chemotherapeutic agent via a linker including a cleavable peptide portion which may be useful with the present invention is shown in Formula CXXVII.

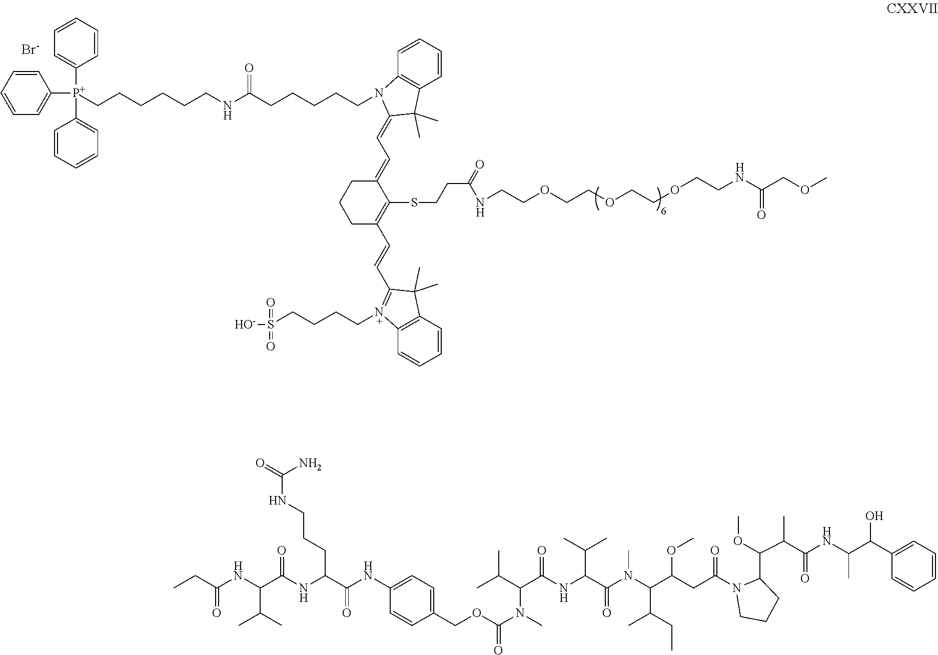

Exemplary conjugates comprising a tumor-targeting NIR dye having a mitochondria-targeting functional group and specifically, the dye shown in Formula CXXIV, coupled to a chemotherapeutic agent via a linker including a cleavable peptide portion which may be useful with the present invention are shown in Formulas CXXVIII and CXXIX.

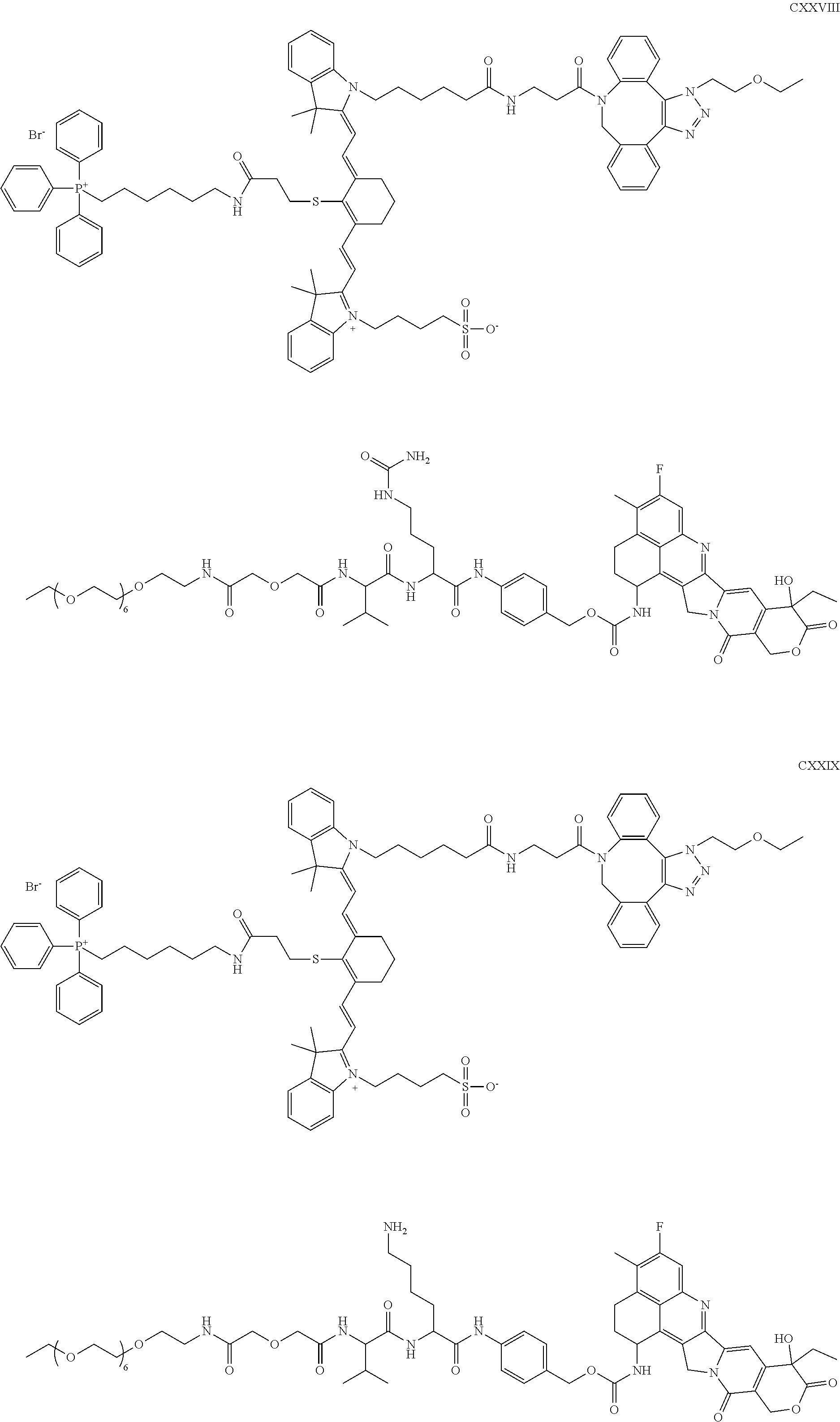

As to particular embodiments, as stated above, the linker can also be modified to have a mitochondria-targeting functionality, whereby such a modification may include the attachment of one or more lipophilic moieties and/or cationic moieties to the linker or the incorporation of one or more lipophilic moieties and/or cationic moieties into the linker.

Exemplary linkers having a mitochondria-targeting functional group and a cleavable peptide portion which may be useful with the present invention are shown in Formulas CXXX through CXXXII.

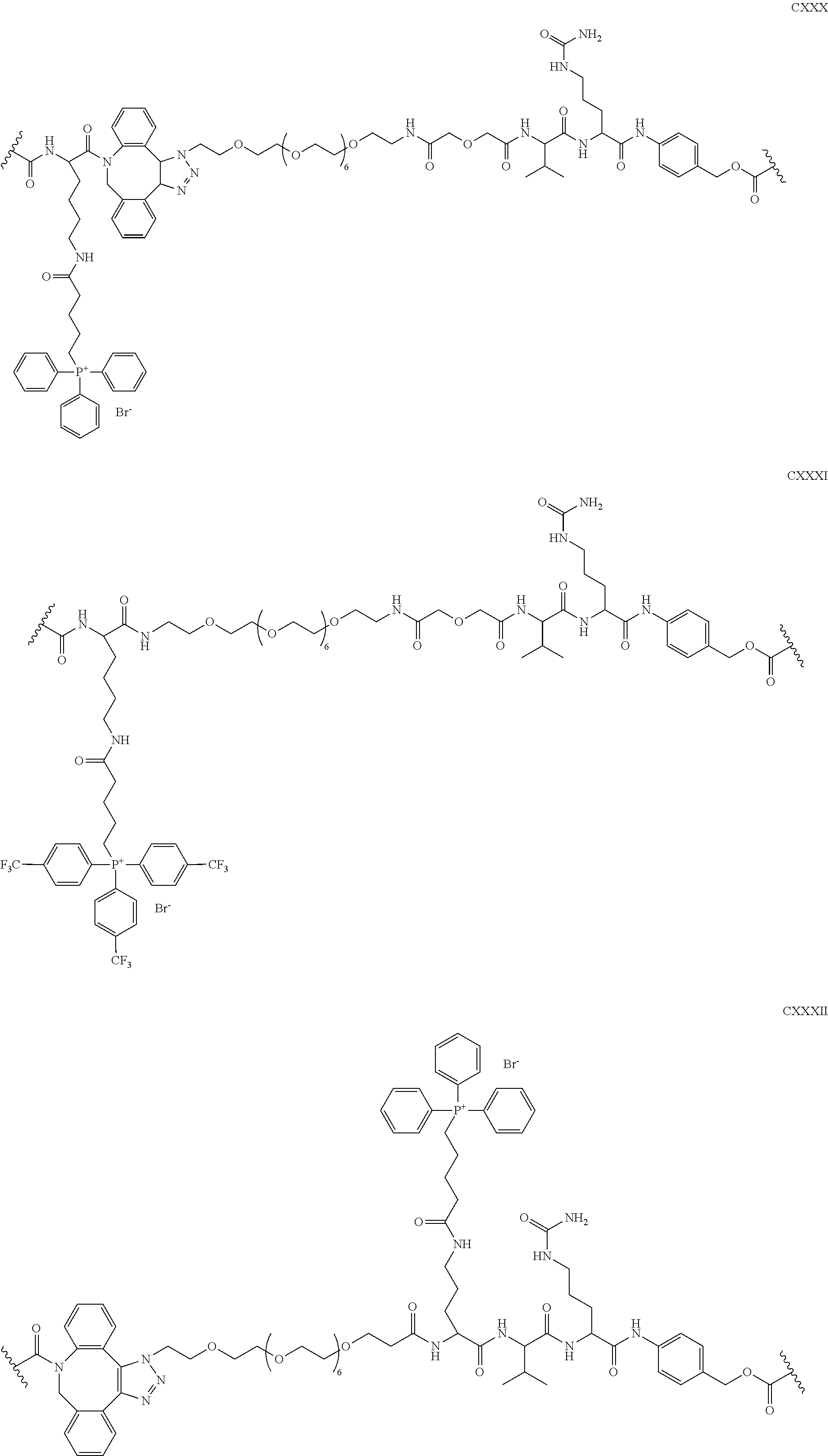

An exemplary conjugate comprising a tumor-targeting NIR dye, a linker having (i) a mitochondria-targeting functional group and specifically, the linker shown in Formula CXXXII, and (ii) a cleavable peptide portion coupled to a chemotherapeutic agent which may be useful with the present invention is shown in Formulas CXXXIII.

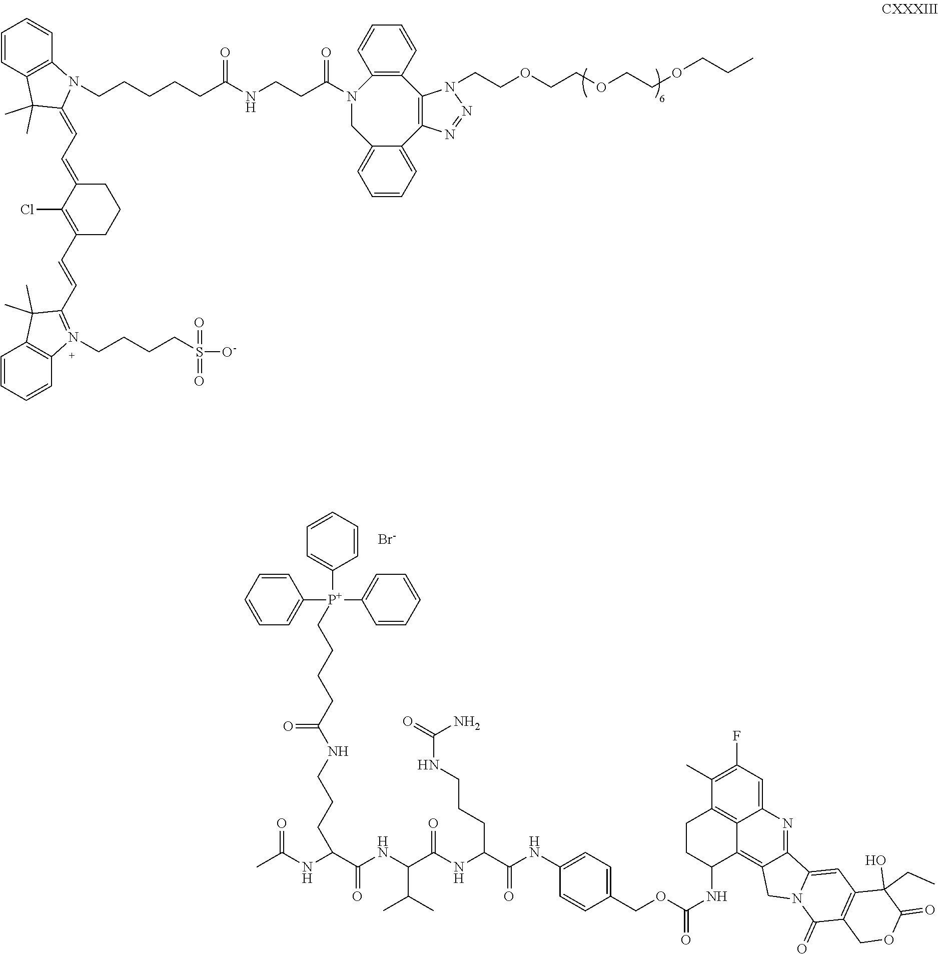

As to particular embodiments, the tumor-targeting NIR dye can form a complex with albumin and such conjugates may be useful for NIR bioimaging, as albumin binding can enhance the brightness, photostability, and biosafety of the dye and correspondingly, enable long-term, high resolution imaging in the NIR window.

As to particular embodiments, the dye can covalently interact with albumin via a meso-chloride which may form a covalent adduct with albumin's free thiol.

Exemplary conjugates comprising a tumor-targeting NIR dye having a meso-chloride coupled to a chemotherapeutic agent via a linker including a cleavable peptide portion which may be useful with the present invention are shown in Formulas CXXXIV and CXXXV.

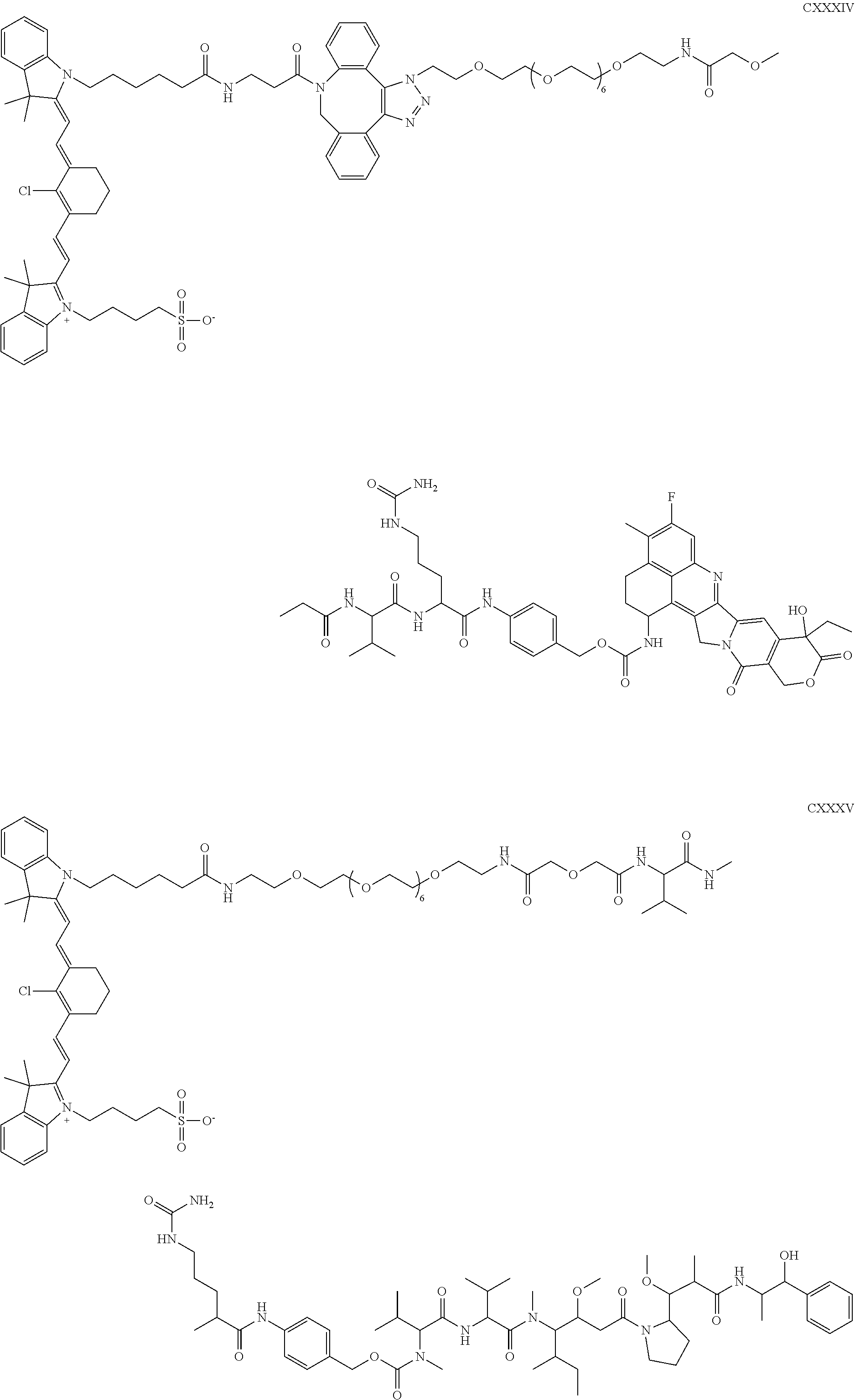

As to other particular embodiments, the dye can noncovalently interact with albumin, such as via electrostatic interactions, hydrophobic interactions, or hydrogen bonding.

As illustrative examples, functional groups which may facilitate noncovalent interactions between the dye and albumin and thus can be useful with the present invention may include those shown in FIGS. 19A and 19B.

An exemplary conjugate comprising a tumor-targeting NIR dye having a fatty acid functional group coupled to a chemotherapeutic agent via a linker including a cleavable peptide portion which may be useful with the present invention is shown in Formula CXXXVI.

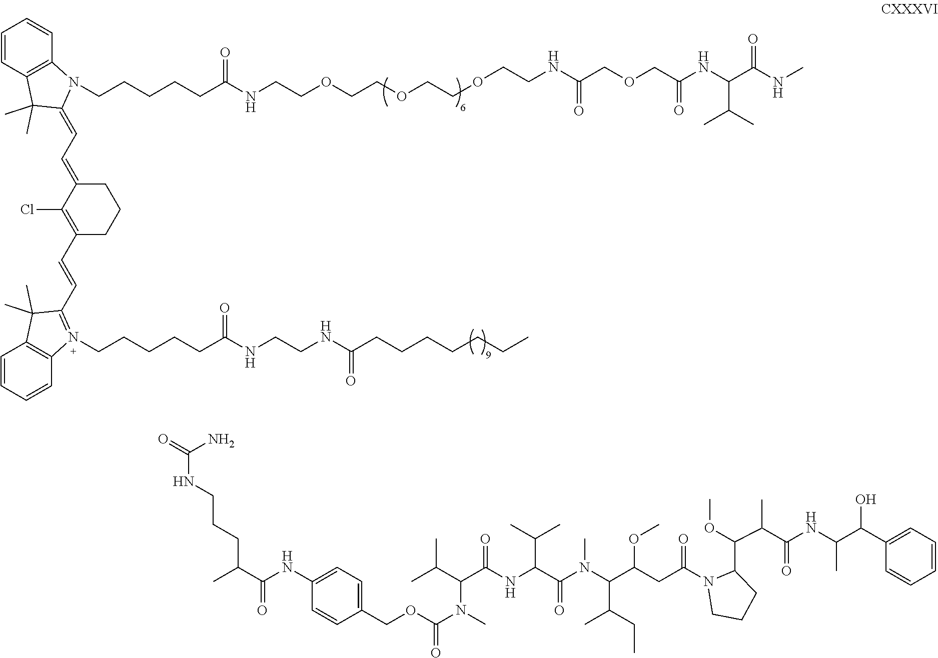

Exemplary conjugates comprising a tumor-targeting NIR dye having a p-iodophenyl butyric acid functional group coupled to a chemotherapeutic agent via a linker including a cleavable peptide portion which may be useful with the present invention are shown in Formulas CXXXVII and CXXXVIII.

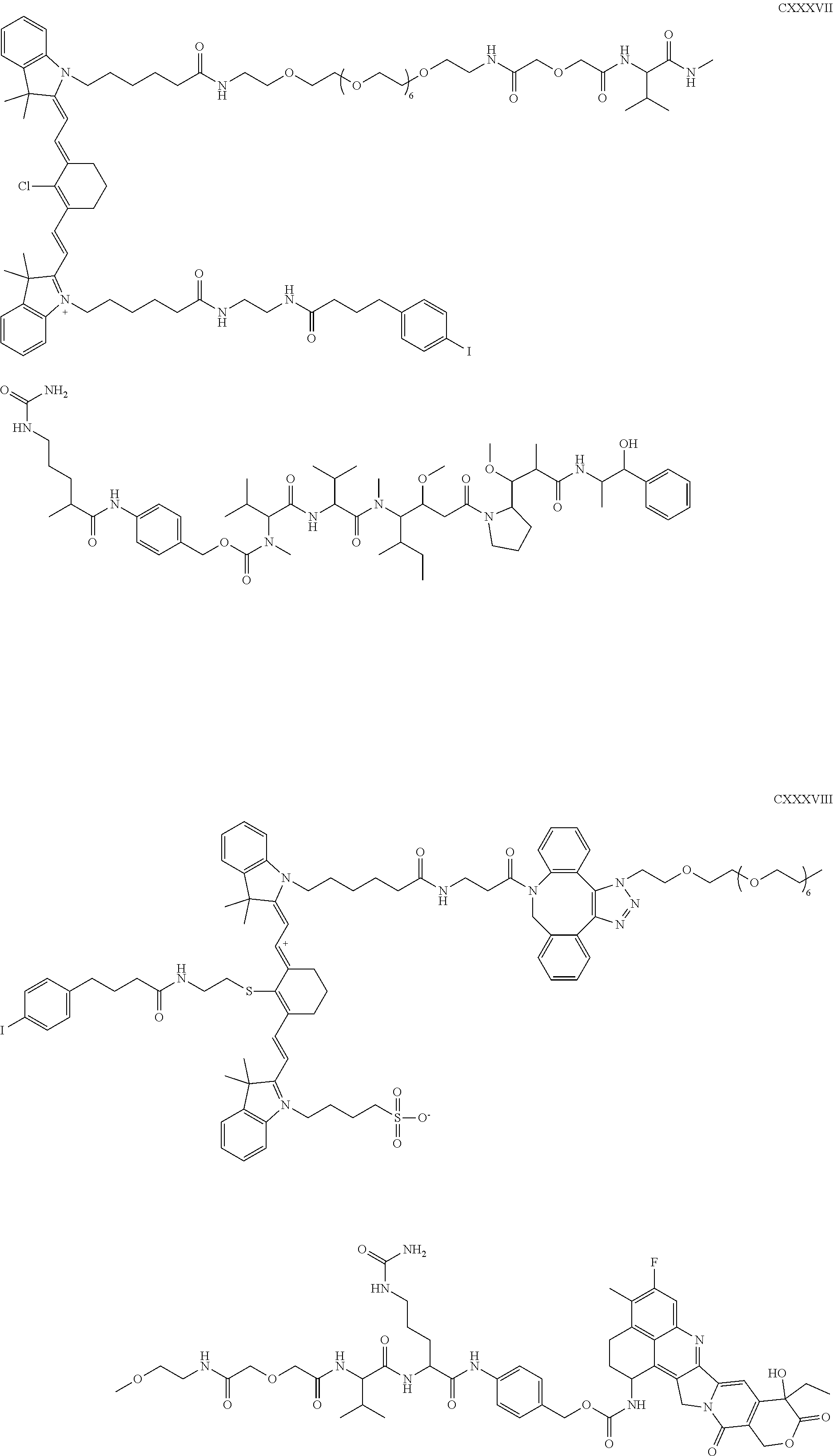

Now referring primarily to FIG. 20, as stated above, the present invention can combine a tumor-targeting NIR dye with various therapeutic agents to create conjugates for the treatment of different cancers, whereby these conjugates may be delivered directly to a target tumor cell population. As to particular embodiments, a dye can be coupled to (for example via conjugation) a payload comprising a radioisotope which may release alpha particles, beta particles, or gamma rays, whereby the dye functions to direct the conjugate and correspondingly, the radioisotope, to a target tumor cell population; this may be in contrast to conventional radiation therapy which can cause significant adverse side effects.

Exemplary radioisotopes which may be useful with the present invention include iodine-131 (¹³¹I), lutetium-177 (¹⁷⁷Lu), yttrium-90 (⁹⁰Y), radium-223 (²²³Ra), actinium-225 (²²⁵Ac), strontium-89 (⁸⁹Sr), samarium-153 (¹⁵³Sm), rhenium-186 (¹⁸⁶Re), rhenium-188 (¹⁸⁸Re), copper-67 (⁶⁷Cu), astatine-211 (²¹¹At), terbium-161 (¹⁶¹Tb), gallium-68 (⁶⁸Ga), americium-241 (²⁴¹Am), and plutonium-239 (²³⁹Pu).

As to particular embodiments, a chelator can be coupled to the radioisotope to form a stable complex; following, the dye can be coupled to the chelator to form the present conjugate including the radioisotope. In practice, due to the short lifespan of radioisotopes, the dye can typically be coupled to the chelator first followed by coupling of the radioisotope to form the present conjugate.

Exemplary chelators which may be useful with the present invention include those shown in FIGS. 21A through 21D, whereby DOTA can be used with ²²⁵Ac and ¹⁷⁷Lu, NOTA can be used with ⁶⁸Ga and ⁹⁰Y, DTPA can be used with ²⁴¹Am and ²³⁹Pu, and macropa can be used with ²²⁵Ac and ²²³Ra.

The dye can be coupled to the chelator to form the present conjugate including the radioisotope via a linker, whereby the linker need not be cleavable. As to particular embodiments, the linker can include a solubility-modifying portion. As to particular embodiments, the linker can include a hydrophilic portion. As to particular embodiments, an illustrative example of a linker including a hydrophilic portion which may be useful with the present invention includes PEG with various chain lengths.

Exemplary conjugates comprising a tumor-targeting NIR dye coupled to a radioisotope via a linker which may be useful with the present invention are shown in Formulas CXXXIX through CXLIV.

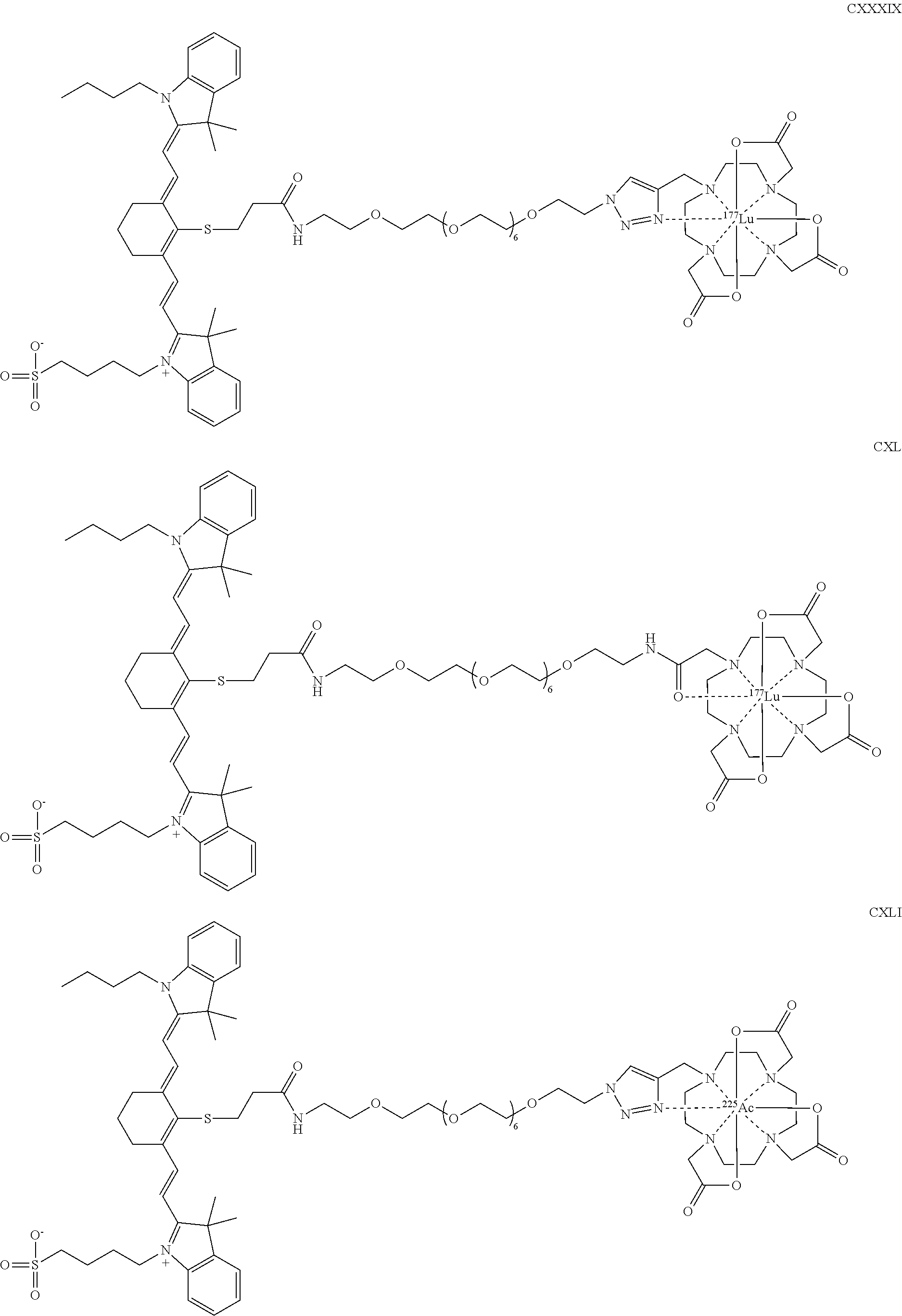

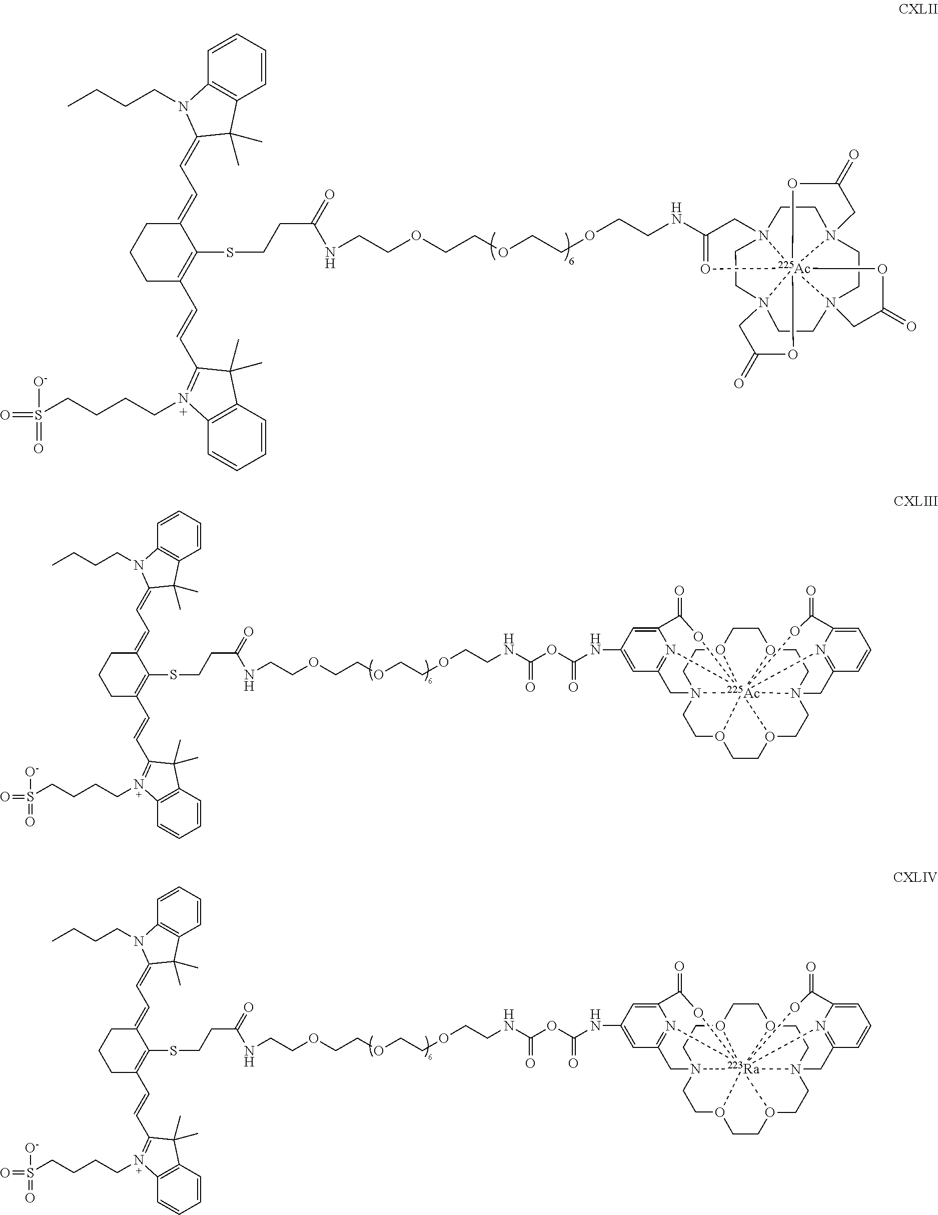

Exemplary conjugates comprising a tumor-targeting NIR dye coupled to (i) a radioisotope via a linker and (ii) a chemotherapeutic agent via a linker including a cleavable peptide portion which may be useful with the present invention are shown in Formulas CXLV and CXLVI.

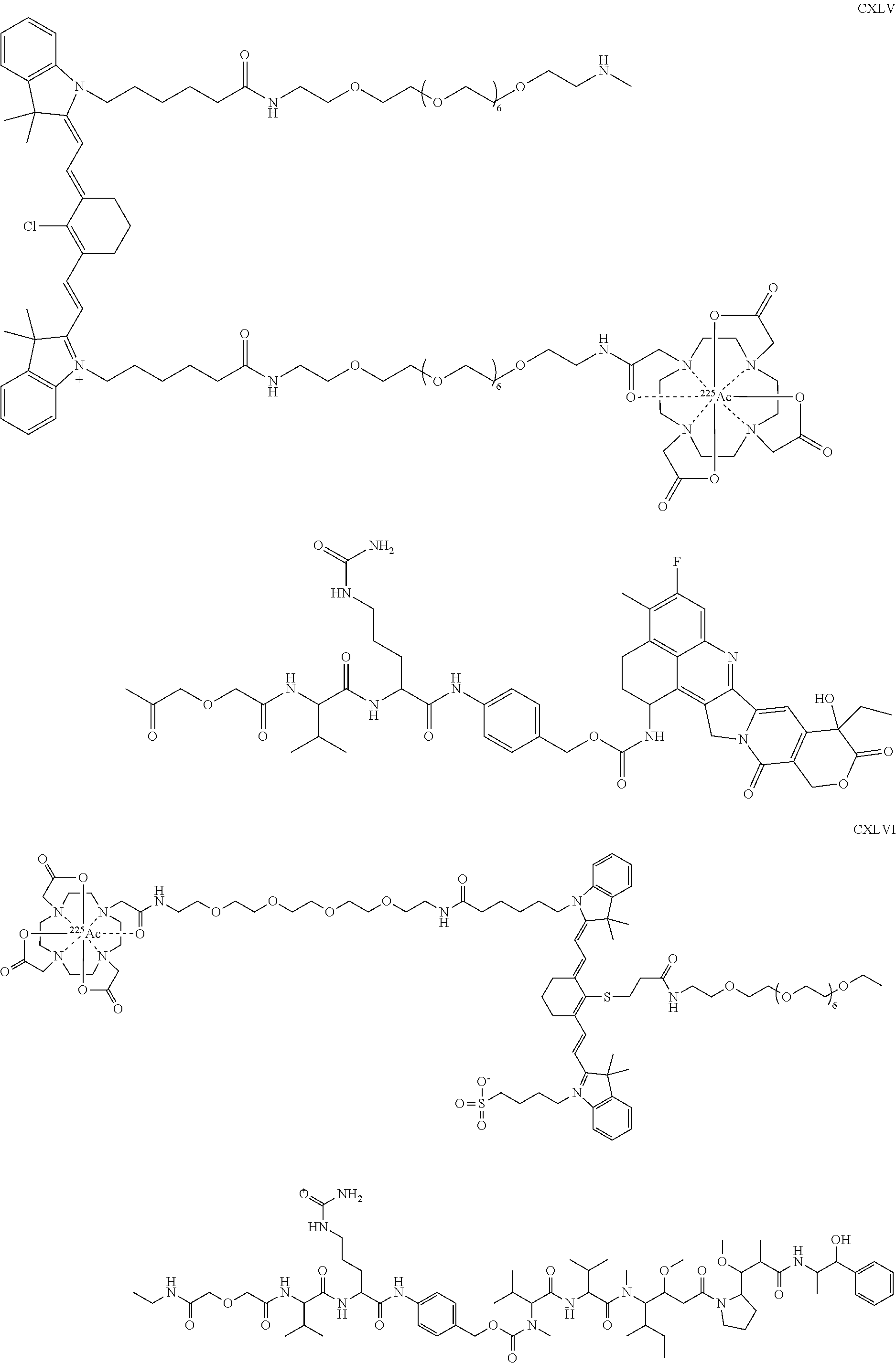

Now referring primarily to FIG. 22, as stated above, the present invention can combine a tumor-targeting NIR dye with various therapeutic agents to create conjugates for the treatment of different cancers, whereby these conjugates may be delivered directly to a target tumor cell population. As to particular embodiments, a dye can be coupled to (for example via conjugation) a payload comprising a small molecule protein degrader, whereby the dye functions to direct the conjugate and correspondingly, the protein degrader, to a target tumor cell population; this may be in contrast to conventional protein degraders which can cause significant adverse side effects.

A protein degrader can induce degradation of a target disease-causing protein, such as by locating said protein proximate E3 ubiquitin ligase which tags said protein with ubiquitin. Upon ubiquitination, the protein can be recognized by a proteasome for degradation into small peptides and amino acids.

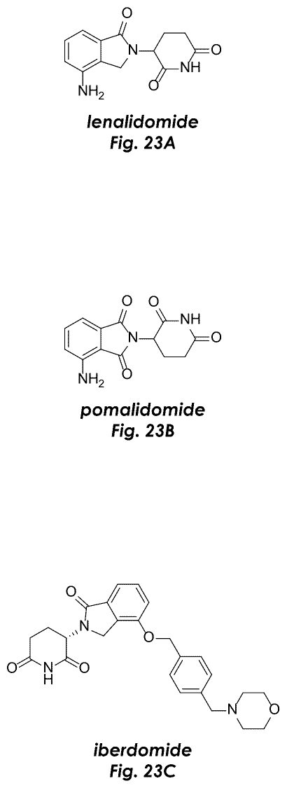

Exemplary small molecule protein degraders which may be useful with the present invention include proteolysis-targeting chimeras (PROTACs) and molecular glues, whereby examples of the latter can include lenalidomide, pomalidomide, and iberdomide as shown in FIGS. 23A through 23C.

Exemplary conjugates comprising a tumor-targeting NIR dye coupled to a molecular glue via a linker including a cleavable peptide portion which may be useful with the present invention are shown in Formulas CXL VII through CL.

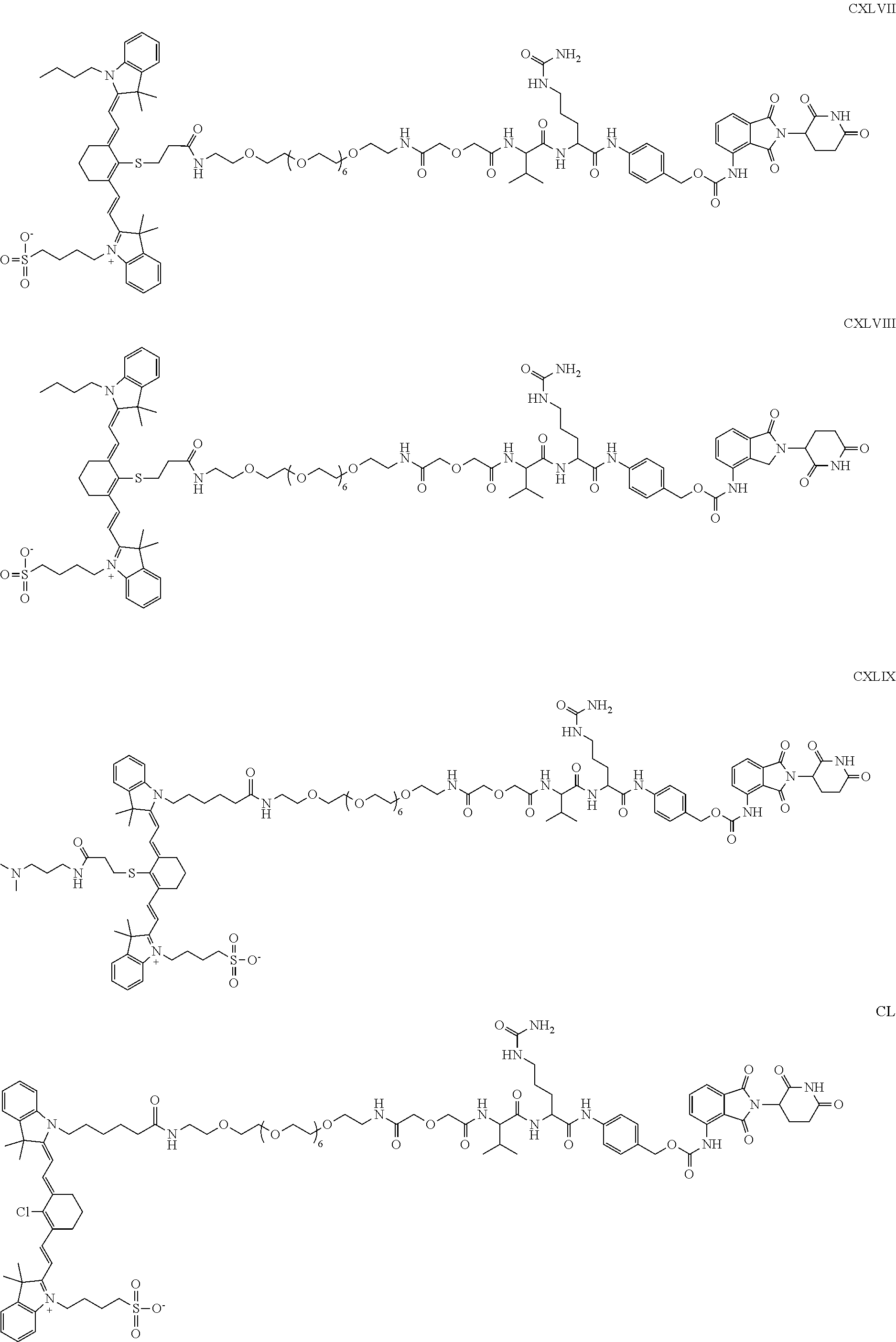

The inventive conjugate structures are shown, described, and claimed without specifying the three-dimensional arrangement at any stereocenter and are intended to be construed as covering all stereoisomers, conformational isomers, and tautomers. Without sacrificing the breadth of the foregoing, particular embodiments of the inventive conjugate structures shown, described, and claimed can include steric features, and specifically can include one or more of the steric features of (i) the protease-sensitive cleavable portions shown in FIGS. 8A through 8N, (ii) the chemotherapeutic agents shown in FIGS. 2A through 4M, and/or (iii) the molecular glues shown in FIGS. 23A through 23C, whereby each of these steric features shown in the Figures is hereby incorporated by reference into each conjugate structure in the description and the claims herein.

### Synthesis

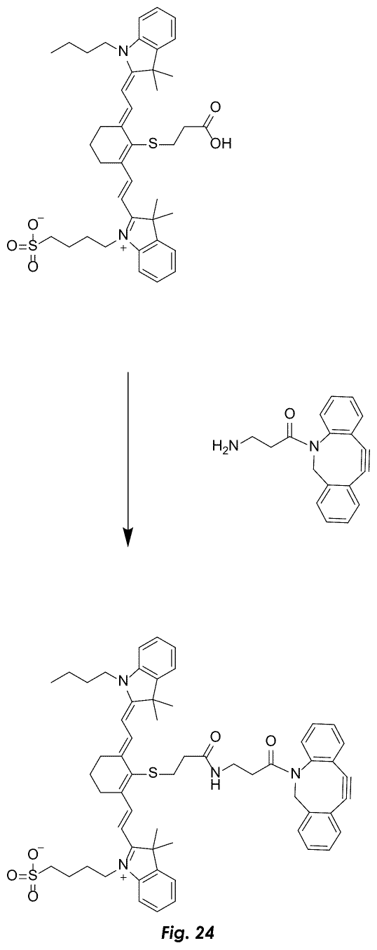

Now referring primarily to FIG. 24, the tumor-targeting NIR dye can be conjugated to DBCO as follows: 1-hydroxybenzotriazole (HOBt) hydrate (2 eq.), 1-ethyl-3-(3-dimethylaminopropyl) carbodiimide hydrochloride (EDC·HCl) (1.6 eq.), and DBCO amine (1.0 eq.) can be added to a solution of dye (1 eq.) in dry dimethylformamide (DMF), such as the dye shown in Formula VI, and the mixture may be stirred for 20 hours (h) at room temperature. The mixture can be concentrated in a vacuum, and the residue may be suspended in dichloromethane (DCM). The mixture can be washed with saturated ammonium chloride (NH₄Cl) and brine, dried over sodium sulfate (Na₂SO₄), and concentrated. The crude product can be purified with a silicon dioxide (SiO₂) column (using a mixture of DCM and methanol (MeOH)) to yield the final product.

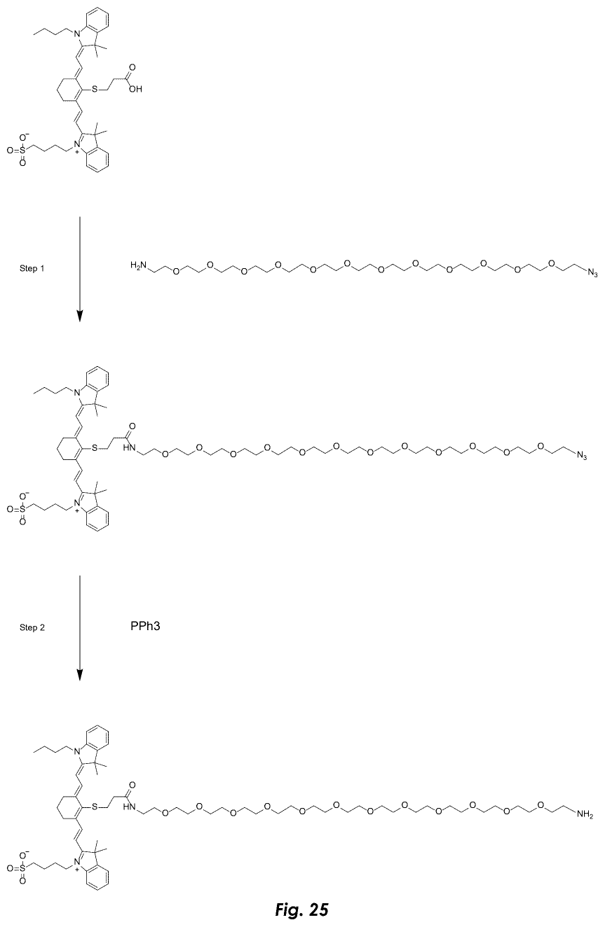

Now referring primarily to FIG. 25, the tumor-targeting NIR dye can be conjugated to PEG as follows.

Step 1: HOBt hydrate (1.2 eq.) and EDC·HCl (1.2 eq.) can be added to a solution of dye (1 eq.) in dry DMF, such as the dye shown in Formula VI. Azido-PEG12-amine (1 eq.) can be added and the mixture may be stirred for 20 h at room temperature. The mixture can be concentrated in a vacuum, and the residue may be suspended in DCM. The mixture can be washed with saturated NH₄Cl and brine, dried over Na₂SO₄, and concentrated. The crude product can be purified with a SiO₂ column (using a mixture of DCM and methanol MeOH) to yield azide-functionalized NIR dye.

Step 2: the product obtained from Step 1 (1 eq.) in tetrahydrofuran (THF), triphenylphosphine (2 eq.), and water (10 eq.) can be combined, and the mixture may be gently heated for a few hours. The reaction can be monitored via thin layer chromatography (TLC). After cooling the mixture to room temperature, DCM can be added and the organic layer may be washed with saline and dried over Na₂SO₄. The crude product can be purified with a SiO₂ column to yield the final product.

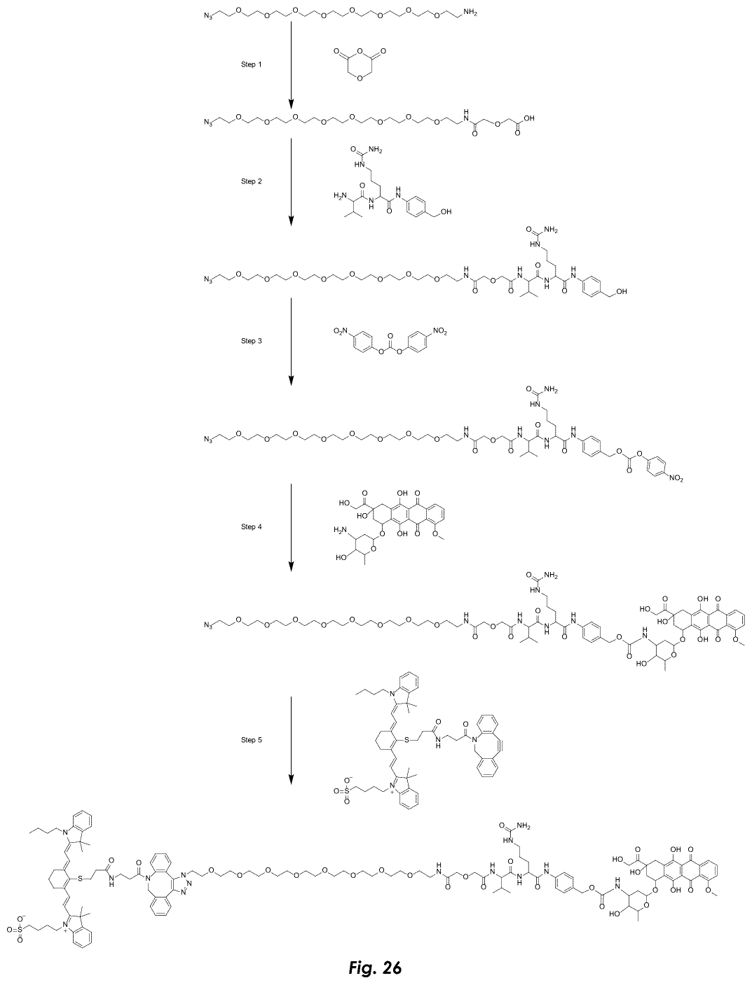

Now referring primarily to FIG. 26, the conjugate shown in Formula XXII can be synthesized as follows.

Step 1: a solution of N₃-(PEG)₈-NH₂ (1 eq) in DCM and diglycolic anhydride (1.5 eq) can be combined, and the mixture may be stirred overnight. The mixture can be concentrated to provide a yellowish residue which may be dissolved in water. The product can be isolated from the aqueous phase by continuous extraction with DCM overnight.

Step 2: the product obtained from Step 1 (1.1 eq), valine-citrulline-PABA (1,000 mg, 2.635 mmol, 1 eq) dissolved in DMF, N,N-diisopropylethylamine (DIPEA) (1.1 eq), and hexafluorophosphate benzotriazole tetramethyl uronium (HBTU) (1.1 eq) can be combined, and the mixture may be stirred overnight. The crude product can be purified with a SiO₂ column (using a mixture of DCM and MeOH) to yield the product.

Step 3: the product obtained from Step 2 (1.0 eq.), bis(4-nitrophenyl) carbonate (1.0 eq.), and DIPEA (4.0 eq.) in DMF can be combined, and the mixture may be stirred for 1 h at room temperature. The mixture can be poured into ice-cold ether, and the filtrate may be collected via centrifugation and dried under vacuum to yield the product (a yellowish powder).

Step 4: the product obtained from Step 3 (1.2 eq.) in dimethyl sulfoxide (DMSO), doxorubicin (1.0 eq.) in DMSO, and HOBt hydrate in DMSO (1.0 eq.) can be combined. DIPEA (3.0 eq.) can be added, and the mixture may be stirred for 3 h at room temperature. After consumption of the doxorubicin starting material (monitored via TLC), a mixture of acetonitrile and water can be added. The crude product can be purified via preparative high-performance liquid chromatography (HPLC) to yield the product.

Step 5: the product obtained from Step 4 (1.5 eq.) in a mixture of acetonitrile and water and DBCO-functionalized NIR dye (1 eq.), such as that synthesized in FIG. 24, can be combined, and the mixture may be stirred overnight at 4° C. The mixture can be concentrated in a vacuum, and the crude product may be purified with a SiO₂ column (using a mixture of DCM and MeOH) to yield the conjugate shown in Formula XXII.

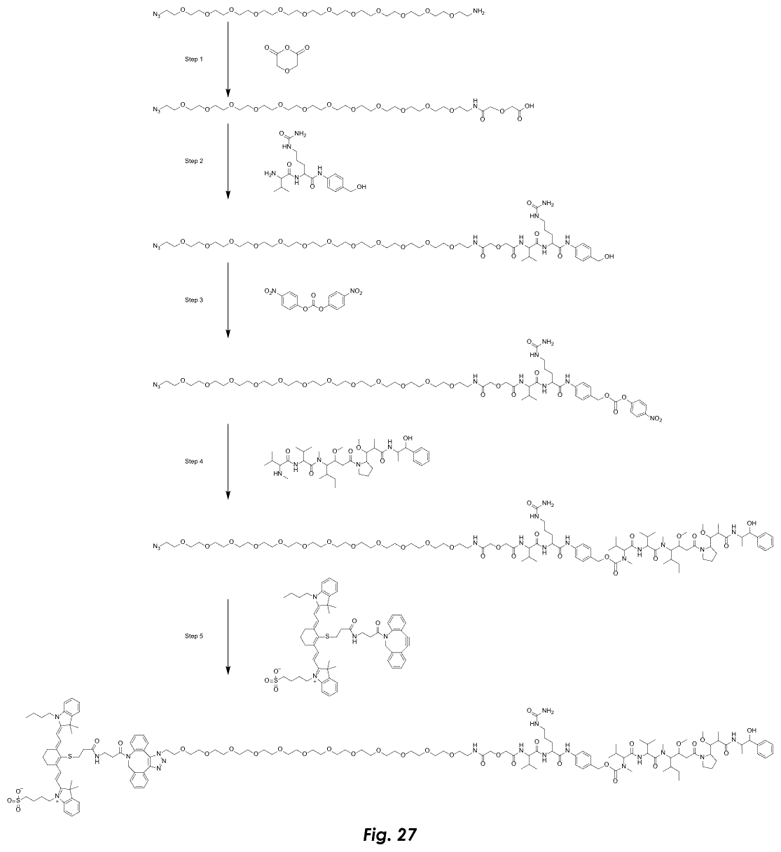

Now referring primarily to FIG. 27, the conjugate shown in Formula XXXIV can be synthesized as follows.

Step 1: a solution of N₃-(PEG)₁₂-NH₂ (1 eq), such as that synthesized in FIG. 25, in DCM and diglycolic anhydride (1.5 eq) can be combined, and the mixture may be stirred overnight. The mixture can be concentrated to provide a yellowish residue. The product can be isolated from the aqueous phase by continuous extraction with DCM overnight.

Step 2: the product obtained from Step 1 (1.1 eq), valine-citrulline-PABA (1,000 mg, 2.635 mmol, 1 eq) dissolved in DMF, DIPEA (1.1 eq), and HBTU (1.1 eq) can be combined, and the mixture may be stirred overnight. The crude product can be purified with a SiO₂ column (using a mixture of DCM and MeOH) to yield the product.

Step 3: the product obtained from Step 2 (1.0 eq.), bis(4-nitrophenyl) carbonate (1.0 eq.), and DIPEA (4.0 eq.) in DMF can be combined, and the mixture may be stirred for 1 h at room temperature. The mixture can be poured into ice-cold ether, and the filtrate may be collected via centrifugation and dried under vacuum to yield the product (a yellowish powder).

Step 4: the product obtained from Step 3 (1.2 eq.) in DMSO, MMAE (1.0 eq.) in DMSO, and HOBt hydrate in DMSO (1.0 eq.) can be combined. DIPEA (3.0 eq.) can be added, and the mixture may be stirred for 3 h at room temperature. After consumption of the MMAE starting material (monitored via TLC), a mixture of acetonitrile and water can be added. The crude product can be purified via preparative HPLC to yield the product.

Step 5: the product obtained from Step 4 (1.5 eq.) in a mixture of acetonitrile and water and DBCO-functionalized NIR dye (1 eq.), such as that synthesized in FIG. 24, can be combined, and the mixture may be stirred overnight at 4° C. The mixture can be concentrated in a vacuum, and the crude product may be purified with a SiO₂ column (using a mixture of DCM and MeOH) to yield the conjugate shown in Formula XXXIV.

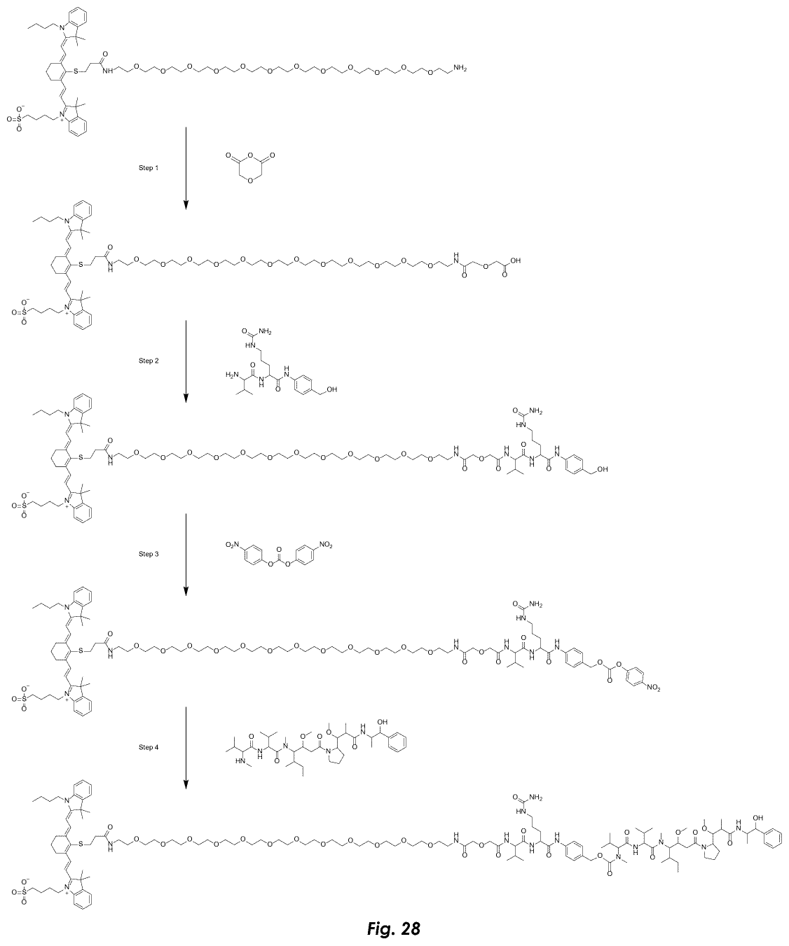

Now referring primarily to FIG. 28, the conjugate shown in Formula LXXII can be synthesized as follows.

Step 1: a solution of dye-(PEG)₁₂-NH₂ (1 eq) in DCM and diglycolic anhydride (1.5 eq) can be combined, and the mixture may be stirred overnight. The crude product can be purified with a SiO₂ column (using a mixture of DCM and MeOH) to yield the product.

Step 2: the product obtained from Step 1 (1.1 eq), valine-citrulline-PABA (1,000 mg, 2.635 mmol, 1 eq) dissolved in DMF, DIPEA (1.1 eq), and HBTU (1.1 eq) can be combined, and the mixture may be stirred overnight. The crude product can be purified with a SiO₂ column (using a mixture of DCM and MeOH) to yield the product.

Step 3: the product obtained from Step 2 (1.0 eq.), bis(4-nitrophenyl) carbonate (1.0 eq.), and DIPEA (4.0 eq.) in DMF can be combined, and the mixture may be stirred for 1 h at room temperature. The mixture can be poured into ice-cold ether, and the filtrate may be collected via centrifugation and dried under vacuum to yield the product.

Step 4: the product obtained from Step 3 (1.2 eq.) in DMSO, MMAE (1.0 eq.) in DMSO, and HOBt hydrate in DMSO (1.0 eq.) can be combined. DIPEA (3.0 eq.) can be added, and the mixture may be stirred for 3 h at room temperature. After consumption of the MMAE starting material (monitored via TLC), a mixture of acetonitrile and water can be added. The crude product can be purified via preparative HPLC to yield the conjugate shown in Formula LXXII.

### Example 1

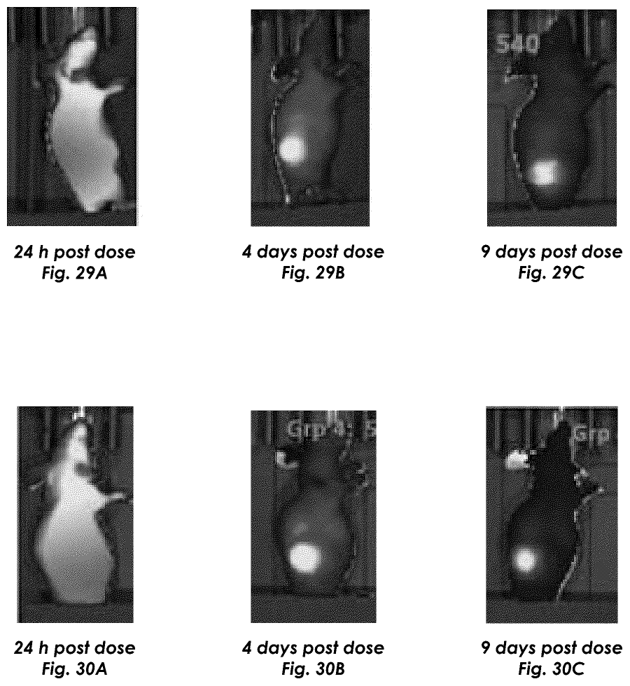

The tumor-targeting ability of the conjugate shown in Formula XXII is presented in FIGS. 29A through 29C, whereby mice bearing tumors derived from the injection of SW620 colon cancer cells into their right flank were subjected to NIR imaging at 24 hours post conjugate dose (via intravenous tail vein injection) (FIG. 29A), 4 days post conjugate dose (FIG. 29B), and 9 days post conjugate dose (FIG. 29C). The results indicate that the conjugate was distributed throughout the body at 24 h post dose, and then was localized to the tumor at 4 and 9 days post dose.

### Example 2

The tumor-targeting ability of the conjugate shown in Formula XLVI is presented in FIGS. 30A through 30C, whereby mice bearing tumors derived from the injection of SW620 colon cancer cells into their right flank were subjected to NIR imaging at 24 hours post conjugate dose (via intravenous tail vein injection) (FIG. 30A), 4 days post conjugate dose (FIG. 30B), and 9 days post conjugate dose (FIG. 30C). The results indicate that the conjugate was distributed throughout the body at 24 h post dose, and then was localized to the tumor at 4 and 9 days post dose.

### Example 3

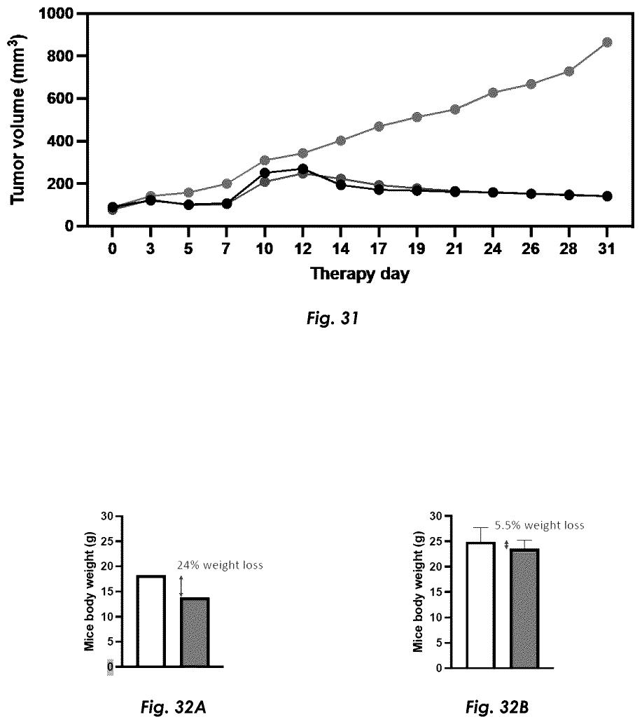

The efficacy of the conjugate shown in Formula XXII is presented in FIG. 31, whereby 3 randomized groups of mice bearing tumors derived from the injection of A549 lung cancer cells into their right flank were dosed (via intravenous tail vein injection) twice a week for 5 weeks with (i) vehicle at 4 mL/kg (Group 1, n=10), (ii) doxorubicin HCl at 3.9 mg/kg (Group 2, n=9), or (iii) the conjugate shown in Formula XXII at 30 mg/kg (Group 3, n=7). Tumor size was measured with digital calipers 3 times a week. In FIG. 31, (i) the average tumor volume of Group 1 is shown in blue, (i) the average tumor volume of Group 2 is shown in black, and (iii) the average tumor volume of Group 3 is shown in red. The results indicate that tumor growth was inhibited in both Groups 2 and 3, particularly after day 12, while tumor growth progressed in Group 1.

### Example 4

The toxicity of the conjugate shown in Formula XXII relative to doxorubicin HCl is presented in FIGS. 32A and 32B in terms of percent body weight loss, whereby during the study of Example 3, the body weight of the mice was recorded at (i) 22 days (when the cumulative dose of doxorubicin was 240 mg) for Group 2, and (ii) 28 days (when the cumulative dose of doxorubicin was 240 mg) for Group 3. The results indicate that toxicity in terms of body weight loss was 4.4 fold less in Group 3 relative to Group 2.

As can be easily understood from the foregoing, the basic concepts of the present invention may be embodied in a variety of ways. The invention involves numerous and varied embodiments of tumor-targeting NIR dyes and associated conjugates and methods for making and using such dyes and associated conjugates.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “therapeutic” should be understood to encompass disclosure of the act of “providing therapy”—whether explicitly discussed or not—and, conversely, were there effectively disclosure of the act of “providing therapy”, such a disclosure should be understood to encompass disclosure of an “therapeutic” and even a “means for providing therapy.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the NIR dyes and associated conjugates herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1-103. (canceled)

104. A composition comprising:
a conjugate comprising:
a tumor-targeting near-infrared dye; and
a therapeutic agent coupled to said dye via a linker comprising a cleavable portion comprising protease sensitivity;
wherein said dye targets said therapeutic agent to tumor cells.

105. The composition of claim 104, wherein said dye comprises Formula VI or Formula VII.

106. The composition of claim 105, wherein said therapeutic agent comprises a chemotherapeutic agent comprising doxorubicin, exatecan, monomethyl auristatin E, or SN-38.

107. The composition of claim 106, wherein said cleavable portion comprises a valine-citrulline dipeptide, a valine-lysine dipeptide, or a glutamic acid-valine-citrulline tripeptide.

108. The composition of claim 107, wherein said linker comprises a self-immolative portion comprising para-aminobenzyl carbamate.

109. The composition of claim 108, wherein said linker comprises a hydrophilic portion comprising PEG.

110. The composition of claim 104, wherein said conjugate comprises Formula XX.

111. The composition of claim 104, wherein said conjugate comprises Formula XXII.

112. The composition of claim 104, wherein said conjugate comprises Formula XXV.

113. The composition of claim 104, wherein said conjugate comprises Formula XXVII.

114. The composition of claim 104, wherein said conjugate comprises Formula XXXIII.

115. The composition of claim 104, wherein said conjugate comprises Formula XXXIV.

116. The composition of claim 104, wherein said conjugate comprises Formula XXXV.

117. The composition of claim 104, wherein said conjugate comprises Formula XLVI.

118. The composition of claim 104, wherein said conjugate comprises Formula LVII.

119. The composition of claim 104, wherein said conjugate comprises Formula LXIX.

120. The composition of claim 109, wherein the tumor-targeting component of said conjugate consists of said dye.

121. A method of killing tumor cells, comprising:
administering a conjugate to a subject, said conjugate comprising:
a tumor-targeting near-infrared dye; and
a therapeutic agent coupled to said dye via a linker comprising a cleavable portion comprising protease sensitivity;
wherein said dye targets said therapeutic agent to tumor cells.

122. The method of claim 121, wherein said conjugate comprises Formula XX, Formula XXII, Formula XXV, Formula XXVII, Formula XXXIII, Formula XXXIV, Formula XXXV, Formula XLVI, Formula LVII, or Formula LXIX.

123. The method of claim 121, wherein said conjugate is selected from the group consisting of Formula XX, Formula XXII, Formula XXV, Formula XXVII, Formula XXXIII, Formula XXXIV, Formula XXXV, Formula XLVI, Formula LVII, and Formula LXIX.
